SEQUENCE LISTING (3 sequences)

SEQ ID NO: 1 - protein, 67 aa, Homo sapiens
PKHEFSVDMTCGGCAEAVSRVLNKLGGVKYDIDLPNKKVCIESEHSMDTLLATLKKTGKTVSYLGLE

SEQ ID NO: 2 - protein, 68 aa, Homo sapiens
CTLEFAVQMTCQSCVDAVRKSLQGVAGVQDVEVHLEDQMVLVHTTLPSQEVQALLEGTGRQAVLKGMG

SEQ ID NO: 3 - protein, 68 aa, Homo sapiens
STTLIAIAGMTCASCVHSIEGMISQLEGVQQISVSLAEGTATVLYNPSVISPEELRAAIEDMGFEASV

(19) United States
(12) Patent Application Publication
(10) Pub. No.: US 2016/0074373 A1
(43) Pub. Date: Mar. 17, 2016

(54) METHODS AND COMPOSITIONS FOR INHIBITING HUMAN COPPER TRAFFICKING PROTEINS ATOX1 AND CCS

(71) Applicants: SHANGHAI INSTITUTE OF MATERIA MEDICA, CHINESE ACADEMY OF SCIENCES, Shanghai (CN); THE UNIVERSTIY OF CHICAGO, Chicago, IL (US)

(72) Inventors: Chuan HE, Chicago, IL (US); Jing WANG, Chicago, IL (US); Hualiang JIANG, Shanghai (CN); Cheng LUO, Shanghai (CN); Hong LIU, Shanghai (CN); Junyan LU, Shanghai (CN)

(73) Assignees: The University of Chicago, Chicago, IL (US); Shanghai Institute of Materia Medica, Chinese Academy of Sciences, Shanghai (CN)

(21) Appl. No.: 14/762,980

(22) PCT Filed: Jan. 23, 2014
(86) PCT No.: PCT/US2014/012793

§ 371 Date: Jul. 23, 2015

Related U.S. Application Data
(60) Provisional application No. 61/755,845, filed on Jan. 23, 2013.

(51) Int. Cl. A61K 31/4365; A61K 31/4355; A61K 31/4375; A61K 31/444

(52) U.S. Cl. CPC A61K 31/4365; A61K 31/444; A61K 31/4355; A61K 31/4375

(57) ABSTRACT

Compositions and methods concern organic molecules that bind to human Atox1 and CCS at the copper trafficking interface of these proteins. This binding suppresses copper trafficking, which leads to inhibition of cancer cell proliferation and tumor growth. In addition to serving as an effective treatment of cancer, these organic molecules inhibit cellular copper uptake and can be used as treatment of disorders of copper metabolism such as Wilson's disease, which is characterized by copper overload, as well as wound healing.

### CROSS-REFERENCE TO RELATED APPLICATION

This application claims the benefit of priority to U.S. Provisional Patent Application Ser. No. 61/755,845 filed on Jan. 23, 2013, which is hereby incorporated by reference in its entirety.

### BACKGROUND OF THE INVENTION

1. Field of the Invention

The present invention relates generally to the fields of medicine, biochemistry, cell biology, organic chemistry, and oncology. More specifically, it concerns methods and compositions for the inhibition of human copper trafficking proteins Atox1 and CCS.

2. Description of Related Art

Copper is an essential trace element in all living organisms and serves as a catalytic and structural cofactor of key metabolic enzymes that regulate physiological processes, including energy generation, iron acquisition, oxygen transport, cellular metabolism, peptide hormone maturation, blood clotting, signal transduction and a host of other processes (Pena et al. 1999, Culotta and Gitlin 2001, Linder 1991).

Evidence suggests that copper plays a significant role in the control of normal endothelial cell growth (Pena et al. 1999, Hu 1998), endothelial cell proliferation in wound-healing (Mandinov et al. 2003), and cancer (Lowndes and Harris, 2005). Since Folkman proposed the concept that the growth and metastasizing capacity of solid tumor is dependent on angiogenesis, a wide array of angiogenic factors have been discovered (Folkman 1971, Brem 1999). Angiogenesis is the process whereby new blood vessels develop from pre-existing vessels and plays a critical role in the transition of tumors from a dormant state to a malignant state (Folkman 1995). Copper has long been recognized as an important factor in the ability of mammals to mount an angiogenic response, and endothelial cells are induced to become more mobile when incubated with copper (Ziche et al. 1982, McAuslan and Reilly 1980). Several angiogenic factors require copper for their secretion, and many copper-dependent enzymes are involved in cell proliferation and migration. However, the mechanism for the role of copper in angiogenesis has yet to be determined (Finney et al. 2009).

Increasing evidence suggests that copper plays fundamental roles in regulating the cell growth involved in various pathophysiologies including tumor growth and neuron degenerative diseases (Pena et al., 1999, Culotta and Gitlin, 2001, Linder, 1991, McAuslan and Gole 1980, Mandinov et al. 2003, Goodman et al. 2004, Brewer 2005, Hu 1998, Volker, et al. 1997, Sen et al. 2002, Birkaya and Aletta 2005, Harris 2004). Notably, cancer cell lesion nuclei have higher copper levels than normal tissues (Daniel et al. 2004, Fuchs and de Lustig, 1989, Arnold and Sasse 1961).

Copper is essential for the activity of both human superoxide dismutase (SOD) and cytochrome c oxidase (CCO). Previous studies have shown that SOD inactivation generates reactive oxygen species (ROS) and oxidative stress inside cells (Huang et al. 2000, Marikovsky et al. 2002). Although the exact mechanism for copper delivery to CCO remains unclear, previous reports have shown that an ATP7A (the copper delivery target of Atox1) defect led to reduced CCO activity (Mercer 1998). Furthermore, ROS induction has been suggested as a critical pathway to inhibit cancer cell proliferation (DeNicola et al. 2011, Raj et al. 2011).

Diseases related to copper excess include Wilson's disease, India childhood cirrhosis, endemic Tyrolean infantile cirrhosis and idiopathic copper toxicosis (Wilson 1912, Tanner 1998, Muller et al. 1996, Muller et al. 1998, Scheinberg and Sternlieb 1996). Copper plays a role in inflammatory disorders as well (Milanino 1993).

Previous studies have suggested that tetrathiomolybdate (TM), an orally active agent for treatment of disorders of copper metabolism, actually inhibits copper uptake through formation of metal clusters (Alvarez et al. 2010) and that TM-induced copper deficiency significantly inhibited tumor growth in severe combined immunodeficient mice (Cox et al. 2001, Brewer et al. 2000, Redman et al. 2003, Khan et al. 2002, Pan et al. 2002).

Organic molecules that bind to the copper trafficking interface of human Atox1 and copper chaperone for superoxide dismutase (CC S) are described. This binding can effectively suppress copper trafficking, which leads to inhibition of cancer cell proliferation and tumor growth. In addition to serving as a potential treatment for cancers, these cellular copper uptake inhibitors also have great potential for the treatment of disorders of copper metabolism such as Wilson's disease, which is characterized by copper overload, as well as wound healing.

### SUMMARY OF THE INVENTION

In a first embodiment there is provided a method of inhibiting copper trafficking of a cell, comprising administering to the cell an effective amount of a small molecule that inhibits the human copper trafficking protein Atox1. In a further embodiment there is provided a method of inhibiting copper trafficking of a cell, comprising administering to the cell an effective amount of a small molecule that inhibits the human copper trafficking protein CCS. In an additional embodiment, there is provided a method of inducing cellular oxidative stress by means of inhibiting copper trafficking. In some embodiments, blocking copper trafficking reduces cellular ATP levels, resulting in activation of AMP-activated protein kinase (AMPK) that leads to lipogenesis.

In specific embodiments, compositions and methods are provided to inhibit human copper chaperone Atox1 and/or CSS. In some embodiments, methods comprising providing to the cell an effective amount of Atox1 and/or CSS inhibitor, wherein the Atox1 and/or CSS inhibitor is a compound of the formula:

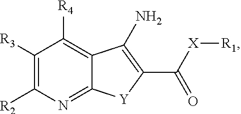

or an enantiomer, racemic mixture, or pharmaceutically acceptable salt thereof, wherein constituent variables are defined herein.

In some embodiments, X is NH, O, CH₂, or S; Y is NH, O or S; R₁ is hydrogen, substituted or unsubstituted C₁-C₆ alkyl, substituted or unsubstituted, saturated or unsaturated C₃-C₁₅ heterocyclic group, substituted or unsubstituted C₆-C₁₀ aromatic group, or substituted or unsubstituted, saturated or unsaturated C₈-C₁₅ condensed ring group; R₂ and R₃ are each independently selected from hydrogen, halogen, substituted or unsubstituted, saturated or unsaturated C₃-C₁₅ heterocyclic group, substituted or unsubstituted C₆-C₁₀ aromatic group, or R₂ and R₃ may join together and form substituted or unsubstituted, saturated or unsaturated C₅-C₇ cycloalkyl group, substituted or unsubstituted, saturated or unsaturated C₃-C₁₅ heterocyclic group, or a substituted or unsubstituted C₆-C₁₀ aromatic group; and, R₄ is hydrogen, halogen, C₁-C₆ alkyl, halogen-substituted C₁-C₆ alkyl, C₁-C₆ alkoxy, halogen-substituted C₁-C₆ alkoxy, cyano, nitro, hydroxyl, substituted amido, or substituted acyl group. Compositions may include this compound or any other compound discussed herein.

In some embodiments, a composition comprises an inhibitor compound having the formula:

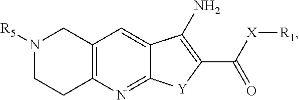

wherein X is NH or O,

Y is O or S,

R₅ is hydrogen, C₁-C₆ alkyl, C₁-C₆ acyl, or —CO₂—C₁-C₆ alkyl, and

R₁ is substituted or unsubstituted, saturated or unsaturated C₃-C₁₅ heterocyclic group, or substituted or unsubstituted C₆-C₁₀ aromatic group. In further embodiments a composition comprises an inhibitor compound having the formula:

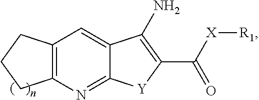

wherein n is 1 or 2,

X is NH or O,

Y is O or S, and

R₁ is substituted or unsubstituted, saturated or unsaturated C₃-C₁₅ heterocyclic group, or substituted or unsubstituted C₆-C₁₀ aromatic group.

Other compositions concern an inhibitor compound having the formula:

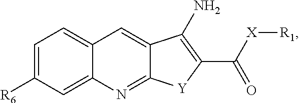

wherein X is NH or O,

Y is O or S,

R₆ is hydrogen, C₁-C₆ alkyl, C₁-C₆ acyl, and C₁-C₆ alkoxy, and

R₁ is substituted or unsubstituted, saturated or unsaturated C₃-C₁₅ heterocyclic group, or substituted or unsubstituted C₆-C₁₀ aromatic group. Other embodiments concern a composition comprising an inhibitor compound having the formula:

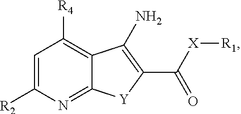

wherein R₂ is substituted or unsubstituted, saturated or unsaturated C₃-C₁₅ heterocyclic group, or substituted or unsubstituted C₆-C₁₀ aromatic group, and

R₁ is C₁-C₆ alkyl, substituted or unsubstituted C₆-C₁₀ aromatic group, and

R₄ is hydrogen, C₁-C₆ alkyl, halogen-substituted C₁-C₆ alkyl, C₁-C₆ alkoxy, halogen-substituted C₁-C₆ alkoxy.

In some embodiments there are compositions comprising an inhibitor compound having the formula:

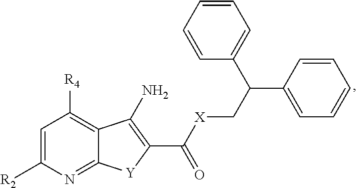

wherein X is NH or O,

Y is O or S,

R₂ is substituted or unsubstituted, saturated or unsaturated C₃-C₁₅ heterocyclic group,
or

substituted or unsubstituted C₆-C₁₀ aromatic group, and

R₄ is a hydrogen, C₁-C₆ alkyl, halogen-substituted C₁-C₆ alkyl, C₁-C₆ alkoxy or halogen-substituted C₁-C₆ alkoxy.

Further compositions and methods concern an inhibitor compound having the formula:

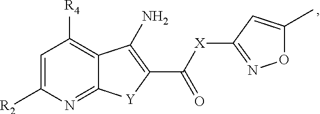

wherein X is NH or O,

Y is O or S,

R₂ is substituted or unsubstituted, saturated or unsaturated C₃-C₁₅ heterocyclic group, or substituted or unsubstituted C₆-C₁₀ aromatic group, and

R₄ is a hydrogen, C₁-C₆ alkyl, halogen-substituted C₁-C₆ alkyl, C₁-C₆ alkoxy or halogen-substituted C₁-C₆ alkoxy.

In some embodiments there is a pharmaceutical composition comprising a compound with the formula:

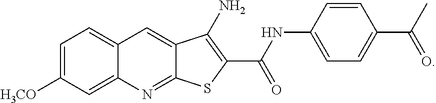 [SMILES: COc1ccc2cc3c(N)c(C(=O)Nc4ccc(C(C)=O)cc4)sc3nc2c1]

In some embodiments, a composition or method concerns an inhibitor compound that is depicted herein or as a comparable ammonium salt thereof.

In certain embodiments, an Atox1 inhibitor binds to the copper trafficking interface of Atox1. In certain aspects of the embodiments, binding of an Atox1 inhibitor directly decreases, inhibits, and/or attenuates Atox1 protein activity when the Atox1 protein is exposed to the compound. In some embodiments, binding of an Atox1 inhibitor to the copper trafficking interface disrupts simultaneous copper binding. In yet further aspects of the embodiments, binding of an Atox1 inhibitor to the copper trafficking interface prevents copper from binding to the interface. in some embodiments, administration of an Atox1 inhibitor inhibits cellular copper uptake. In further embodiments an Atox1 inhibitor binds specifically to the Atox1 protein, that is an Atox1 inhibitor may bind to the Atox1 protein with higher affinity than binding of the Atox1 inhibitor to other proteins, receptors, and/or binding sites. Binding affinity may be measured by displacement of an Atox1-bound radiolabeled ligand by the Atox1 inhibitor. In some embodiments, an Atox1 inhibitor specifically binds Atox1 and/or Atox1-like domains that mediate copper trafficking inside mammalian cells. In certain embodiments, a CCS inhibitor binds to the copper trafficking interface of CCS. In certain aspects of the embodiments, binding of a CCS inhibitor directly decreases, inhibits, and/or attenuates CCS protein activity when the CCS protein is exposed to the compound. In some embodiments, binding of a CCS inhibitor to the copper trafficking interface disrupts simultaneous copper binding. In yet further aspects of the embodiments, binding of a CCS inhibitor to the copper trafficking interface prevents copper from binding to the interface. In some embodiments, administration of a CCS inhibitor inhibits cellular copper uptake. In further embodiments a CCS inhibitor binds specifically to the CCS protein, that is a CCS inhibitor may bind to the CCS protein with higher affinity than binding of the CCS inhibitor to other proteins, receptors, and/or binding sites. Binding affinity may be measured by displacement of a CCS-bound radiolabeled ligand by the CCS inhibitor. In some embodiments, a CCS inhibitor specifically binds CCS and/or CCS-like domains that mediate copper trafficking inside mammalian cells.

In certain embodiments, an Atox1 inhibitor alters the activity of Atox1 directly, and not indirectly, such as by altering expression levels of Atox1. In certain embodiments, a CCS inhibitor alters the activity of CCS directly, and not indirectly, such as by altering expression levels of CCS.

In certain aspects of the embodiments, administration of an Atox1 inhibitor is used to treat diseases related to copper disorders. In certain aspects of the embodiments, administration of an Atox1 inhibitor is used to treat diseases associated with copper excess. In yet further aspects, administration of an Atox1 inhibitor is used to treat Wilson's disease, India childhood cirrhosis, endemic Tyrolean infantile cirrhosis, and/or idiopathic copper toxicosis. In yet further aspects of the invention, administration of an Atox1 inhibitor promotes wound healing. In other embodiments of the invention, an Atox1 inhibitor is used to treat idiopathic copper toxicosis idiopathic pulmonary fibrosis, liver fibrosis, primary biliary cirrhosis, diabetes mellitus, alzheimer's disease, Huntington's disease, amyotrophic lateral sclerosis, multiple sclerosis, inflammation, or an autoimmune disease.

In certain aspects of the embodiments, administration of a CCS inhibitor is used to treat diseases related to copper disorders. In certain aspects of the embodiments, administration of a CCS inhibitor is used to treat diseases associated with copper excess. In yet further aspects, administration of a CCS inhibitor is used to treat Wilson's disease, India childhood cirrhosis, endemic Tyrolean infantile cirrhosis, and/or idiopathic copper toxicosis. In yet further aspects, administration of a CCS inhibitor promotes wound healing. In other embodiments of the invention, a CCS inhibitor is used to treat idiopathic copper toxicosis idiopathic pulmonary fibrosis, liver fibrosis, primary biliary cirrhosis, diabetes mellitus, alzheimer's disease, Huntington's disease, amyotrophic lateral sclerosis, multiple sclerosis, inflammation, or an autoimmune disease.

In still further aspects, administration of an Atox1 inhibitor is used to treat cancer. In certain embodiments, administration of an Atox1 inhibitor is toxic to cancer or tumor cells. In certain embodiments, administration of an Atox1 inhibitor reduces tumor growth and size. In yet further aspects, administration of an Atox1 inhibitor inhibits cancer cell proliferation. In some aspects, administration of an Atox1 inhibitor inhibits angiogenesis.

In a further aspect, administration of a CCS inhibitor is used to treat cancer. In certain embodiments, administration of a CCS inhibitor is toxic to cancer or tumor cells. In certain embodiments, administration of a CCS inhibitor reduces tumor growth and size. In yet further aspects, administration of a CCS inhibitor inhibits cancer cell proliferation. In some aspects, administration of a CCS inhibitor inhibits angiogenesis.

In certain embodiments, there are pharmaceutical compositions for treating and/or preventing cancers, Wilson's disease, India childhood cirrhosis, endemic Tyrolean infantile cirrhosis, idiopathic copper toxicosis idiopathic pulmonary fibrosis, liver fibrosis, primary biliary cirrhosis, diabetes mellitus, alzheimer's disease, huntington's disease, amyotrophic lateral sclerosis, multiple sclerosis, inflammation, and autoimmune diseases comprising a therapeutically effective amount of the compound

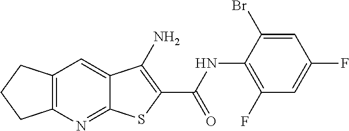 [SMILES: Nc1c(C(=O)Nc2c(F)cc(F)cc2Br)sc2nc3c(cc12)CCC3]

or a pharmaceutically acceptable salt thereof.

Some embodiments provide a method of treating cancer or a tumor comprising administering to a patient a pharmaceutically acceptable composition comprising

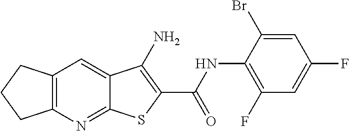 [SMILES: Nc1c(C(=O)Nc2c(F)cc(F)cc2Br)sc2nc3c(cc12)CCC3]

or a pharmaceutically acceptable salt thereof.

In certain aspects of the embodiments, administration of an Atox1 inhibitor induces reactive oxygen species. In other aspects of the embodiments, administration of an Atox1 inhibitor induces oxidative stress. In some aspects of the embodiments, administration of an Atox1 inhibitor induces reactive oxygen species that inhibit cancer cell proliferation. In some aspects of the embodiments, administration of an Atox1 inhibitor induces oxidative stress that inhibits cancer cell proliferation. In certain embodiments, reactive oxygen species induced by an Atox1 inhibitor is toxic to cancer or tumor cells. In certain embodiments, reactive oxygen species induced by an Atox1 inhibitor reduces tumor growth and size.

In some aspects, administration of a CCS inhibitor induces reactive oxygen species. In other aspects of the embodiments, administration of a CCS inhibitor induces oxidative stress. In further aspects of the embodiments, administration of a CCS inhibitor induces reactive oxygen species that inhibit cancer cell proliferation. In other aspects of the embodiments, administration of a CCS inhibitor induces oxidative stress that inhibits cancer cell proliferation. In certain embodiments, reactive oxygen species induced by a CCS inhibitor is toxic to cancer or tumor cells. In certain embodiments, reactive oxygen species induced by a CCS inhibitor reduces tumor growth and size.

In certain embodiments, methods include a step of identifying a patient or subject in need of the CCS or Atox1 inhibitor or in need of treatment for a disease or condition described herein. In other embodiments, methods also include evaluating, determining, measuring, or assessing the efficacy of the inhibitor or its ability to achieve the intended goal as set forth in embodiments discussed herein, such as treating a particular disease or condition or achieving a particular physiological effect.

As used herein the specification, “a” or “an” may mean one or more. As used herein in the claim(s), when used in conjunction with the word “comprising”, the words “a” or “an” may mean one or more than one. The terms “inhibitor”, and “agent” are used interchangeably herein.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Any feature discussed in the context of one embodiment herein is contemplated for use in any other embodiment discussed herein. Accordingly, compositions discussed in the disclosure may be used in any method discussed in the disclosure, and vice versa. Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTIONS OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-1D depicts a copper chaperone transfer mechanism, copper chaperone inhibition and copper trafficking pathways in mammalian cells.

FIG. 2A-2B is a box-plot diagram showing the expression patterns of (A) Atox1 and (B) CCS across diverse tumor and normal tissues. Red represents tumor tissues, and green represents normal tissues.

FIG. 3 is an overview of the screening process.

FIG. 4A is a FRET assay model with an Atox1 antagonist and a copper chelator.

FIG. 4B depicts eCALWY3-based FRET assay in the absence (I) and presence (II) of an inhibitor.

FIG. 4C is a graph of binding curves of compound 50 to Atox1, CCS, and WD4 domain.

FIG. 4D is a thermal shift assay of CCS treated with compound 50.

FIGS. 5A-F depict FRET assay results of the eCALWY3 probe (Atox1 and its copper-binding partner WD4) in response to compounds 2, 30, 49, 50, 61, and 71.

FIG. 6A is a graph of H1299 lung cancer cell viability in response to increasing doses of compounds 2, 30, 49, 50, 61, and 71.

FIG. 6B is a graph of 212LN head and neck cancer cell viability in response to increasing doses of compounds 2, 30, 49, 50, 61, and 71.

FIG. 6C is a graph of MB231 breast cancer cell viability in response to increasing doses of compounds 2, 30, 49, 50, 61, and 71.

FIG. 6D is a graph of human PIG1 immortalized normal melanocyte cells in response to increasing doses of compounds 2, 30, 49, 50, 61, and 71.

FIG. 6E is a graph of human dermal fibroblast cells in response to increasing doses of compounds 2, 30, 49, 50, 61, and 71.

FIG. 7A is a graph of FRET assay of the eCALWY3 probe in the presence of varying concentrations of inhibitor 2.

FIG. 7B is a graph of FRET assay of the eCALWY3 probe in the presence of varying concentrations of inhibitor 50.

FIG. 7C is a graph of FRET assay of the eCALWY3 probe in the presence of varying concentrations of inhibitor 61.

FIG. 7D depicts the dissociation constants (Kd) of compounds 2, 50 and 61 based on FIGS. 6A-6C.

FIG. 8A is an amino acid sequence alignment of human Atox1 (SEQ ID NO. 1), CCS (SEQ ID NO. 2) and WD4 (SEQ ID NO. 3).

FIG. 8B is an overlay of Atox1, CCS and WDR ribbon diagrams.

FIG. 8C depicts the structures of the two Atox1/CCS inhibitors, inhibitor 2 and inhibitor 61.

FIG. 8D is a graph of the intrinsic fluorescent signal changes of tyrosine (Atox1 and WD4) and tryptophan (CCS) with the addition of compound 2.

FIG. 8E is a dose-response graph of a thermal shift assay of CCS treated with compound 2.

FIG. 8F is a graph of the intrinsic fluorescent signal changes of tyrosine (Atox1 and WD4) and tryptophan (CCS) with the addition of compound 61.

FIG. 8G is a thermal shift assay of CCS treated with compound 61.

FIG. 9A illustrates binding curves and Kd values for compound 50 binding to human Atox1 and CCS.

FIG. 9B depicts results of a surface plasmon resonance-based direct binding assay of compound 50 binding to CCS.

FIG. 9C depicts the Kd obtained from the a surface plasmon resonance assay.

FIG. 9D is a thermal shift assay to confirm the stabilization of CCS by compound 50 with a similar Kd value obtained.

FIG. 9E illustrates the interaction of Cu(I)-loaded Atox1 with compound 50 characterized by NMR.

FIG. 10A is a docking model of compound 50 binding to Atox1.

FIG. 10B is a docking model of compound 50 binding to CCS.

FIG. 11A is a docking model of compound 50 binding to Atox1.

FIG. 11B is a FRET dose-fluorescence response graph of compound 50 binding to Atox1 single mutants.

FIG. 11C is a FRET dose-fluorescence response graph of compound 50 binding to Atox1 double mutants.

FIG. 11D is a FRET dose-fluorescence response graph of compound 50 binding to CCS single mutants.

FIG. 11E is a FRET dose-fluorescence response graph of compound 50 binding to CCS double mutants.

FIG. 12 is a model of a genetically-encoded probe for selective copper(I) imaging in living cells, whereby copper(I) binding induces a conformational change of Ace1 which gives rise to decreased intracellular fluorescence.

FIG. 13A is a graph showing that compound 50 reduces cell proliferation in cancer cells.

FIG. 13B is a graph showing that compound 50 does not induce cell death in normal human and pig cells.

FIG. 13C is a Western blot depicting higher expression levels of Atox1 and CCS in cancer cells than in normal cells.

FIG. 13D is a graph showing that H1299 lung cancer cell proliferation decreased when Atox1 and CCS were knocked down.

FIG. 13E shows that inhibition of cell proliferation by compound 50 was rescued with Atox1 and CCS knockdown and knockdown confirmation via Western blotting.

FIG. 14A includes a graph that shows tumor growth and tumor size in xenograft nude mice injected with H1299 lung cancer cells compared to mice treated with compound 50 and pictures of the mice and tumors.

FIG. 14B includes a graph that shows tumor growth and tumor size in xenograft nude mice injected with K562 leukemia cells compared to mice treated with compound 50 and pictures of the mice and tumors.

FIG. 15A is a graph showing that compound 50 does not noticeably affect glucose uptake in H1299 cells.

FIG. 15B is a graph showing that compound 50 does not noticeably affect lactate level in H1299 cells.

FIG. 15C is a graph showing that compound 50 does not noticeably affect RNA synthesis in H1299 cells.

FIG. 15D is a graph showing that compound 50 significantly reduced cellular ATP levels in H1299 cells.

FIG. 15E is a graph showing that compound 50 significantly reduced cellular ATP levels in K562 cells.

FIG. 15F is a graph showing that compound 50 significantly lowers CCO activity in H1299 FIG. 15G is a graph showing that compound 50 significantly lowers CCO activity in K562 cells.

FIG. 16A is a graph showing mitochondrial performance of H1299 cells upon treatment with compound 50, in the presence or absence of ATP synthase inhibitor oligomycin, that resulted in a significant decrease in oxygen consumption rate.

FIG. 16B is a graph showing that compound 50 significantly decreased lipid synthesis in the H1299 cancer cells.

FIG. 16C is a graph showing that compound 50 significantly decreased the NADPH/NADP⁺ ratio in the H1299 cancer cells.

FIG. 16D illustrates that compound 50 increased the levels of AMPK phosphorylation and ACC1 phosphorylation in H1299 and K562 cells.

FIG. 16E-16F shows that of AMPK and ACC1 phosphorylation could not be rescued with the ROS scavenger NAC; however, treatment of an AMPK inhibitor compound C together with compound 50 almost completely reversed the increased phosphorylation on both proteins and recovered lipid synthesis in H1299 cells

FIG. 16G illustrates that decreased lipid synthesis was observed in either Atox1 or CCS knockdown cells.

FIG. 16H is a graph showing that cells that were treated with both NAC and compound C observed almost complete rescue of cell proliferation inhibition induced by compound 50.

FIG. 17 is a mechanistic model of cancer cell proliferation inhibition through targeting copper trafficking proteins Atox1 and CCS that are upregulated in cancer cells.

FIG. 18A is a graph illustrating that treatment of H1299 cells with compound 50 (10 μM) led to increased cellular ROS levels.

FIG. 18B is a graph illustrating that treatment of K562 cells with compound 50 (10 μM) led to increased cellular ROS levels.

FIG. 18C is a graph illustrating that treatment of H1299 cells with compound 50 decreased the ratio of reduced to oxidized glutathione (GSH/GSSG).

FIG. 18D is a graph illustrating that treatment of K562 cells with compound 50 decreased the ratio of reduced to oxidized glutathione (GSH/GSSG).

FIG. 18E illustrates that the increased cellular ROS levels in H1299 cells can be almost completely rescued with the treatment of ROS scavenger N-acetyl-L-cysteine.

FIG. 18F illustrates that the increased cellular ROS levels in K562 cells can be almost completely rescued with the treatment of ROS scavenger N-acetyl-L-cysteine.

FIG. 19A is a graph illustrating that compound 50 noticeably decreased SOD activity in H1299 cells after 48 h treatment. The effect could be reversed by adding CuSO₄ (150 μM).

FIG. 19B is a graph illustrating that compound 50 noticeably decreased SOD activity in K562 cells after 48 h treatment. The effect could be reversed by adding CuSO₄ (150 μM).

FIG. 19C illustrates the effects of that compound 50 on the levels of SOD1 and SOD2 measured by western blotting in H1299 and K562 cells. Reduced expression of SOD2 was observed.

FIG. 20A illustrates 8-OHdG levels in H1299 cancer cells determined by triple quadrupole mass spectrometry analysis. The cellular levels of 8-OHdG increased with compound 50 treatment which could be rescued by NAC (3 mM).

FIG. 20B illustrates 8-OHdG levels in K562 cancer cells determined by triple quadrupole mass spectrometry analysis. The cellular levels of 8-OHdG increased with compound 50 treatment which could be rescued by NAC (3 mM).

FIG. 20C-20D illustrate percentages of each cell cycle for H1299 cancer cells evaluated by flow cytometry in response to different concentrations of compound 50.

FIG. 20E-20F illustrate percentages of each cell cycle for K562 cancer cells evaluated by flow cytometry in response to different concentrations of compound 50.

FIG. 21A is a graph illustrating that compound 50 does not noticeably induce apoptosis in H1299 cells.

FIG. 21B is a graph illustrating that compound 50 does not noticeably affect caspase-3 activity in H1299 cells.

FIG. 22A is a graph illustrating the effects of treatment with 10 μM of compound 50 to Tetrathiomolybdate (TM) and Cisplatin on the rate of H1299 cancer cell proliferation.

FIG. 22B is a graph illustrating the effects of treatment with 10 μM of compound 50, TM and Cisplatin on the rate of K562 cancer cell proliferation.

FIG. 22C is a graph illustrating relative intracellular ATP levels in response to treatment of H1299 cancer cells with compound 50, TM and Cisplatin.

FIG. 22D is a graph illustrating relative intracellular ATP levels in response to treatment of K562 cancer cells with compound 50, TM and Cisplatin.

FIG. 22E is a graph illustrating relative ROS levels in H1299 cancer cells upon treatment with compound 50, TM and Cisplatin.

FIG. 22F is a graph illustrating relative ROS levels in K562 cancer cells upon treatment with compound 50, TM and Cisplatin.

### DESCRIPTION OF ILLUSTRATIVE EMBODIMENTS

Methods and compositions are provided involving small molecules that bind to human Atox1 and CCS at the copper trafficking interface of these proteins. This binding can suppress copper trafficking, which leads to inhibition of cancer cell proliferation and tumor growth. In addition to serving as an effective treatment of cancer, these molecules inhibit cellular copper uptake and can be used as treatment of disorders of copper metabolism such as Wilson's disease, which is characterized by copper overload, as well as wound healing.

In some aspects, an Atox1 inhibitor is administered in conjunction with at least a second anti-cancer therapy. For instance, an Atox1 inhibitor can be administered before, after or essentially simultaneously with said second therapy. Examples of a second anticancer therapy include, without limitation a surgical, radiation, hormonal, cancer cell-targeted or chemotherapeutic anticancer therapy. Methods may involve multiple administrations of one or more compounds, compositions, and/or agents.

In still further aspects, a CCS inhibitor is administered in conjunction with at least a second anti-cancer therapy. For instance, of a CCS inhibitor can be administered before, after or essentially simultaneously with said second therapy. Examples of a second anticancer therapy include, without limitation a surgical, radiation, hormonal, cancer cell-targeted or chemotherapeutic anticancer therapy.

Inhibiting copper trafficking is one way that might be used to treat various human diseases including Wilson's disease, inflammatory disorders, autoimmune diseases, fibrotic diseases, diabetes mellitus, neurogenerative diseases, and cancers.

Certain aspects of the embodiments concern a patient having a cancer. For example the patient can have an oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer. In some aspects, the patient has an epithelial cancer. In yet further aspects, the patient has an endometrial cancer, an ovarian cancer or a melanoma. In further aspects, the patient is a patient that has previously received one or more anti-cancer therapy or has pervious failed to adequately respond to one or more anti-cancer therapy. Thus, in some aspects, the cancer is a cancer that is resistant to at least a first anti-cancer therapy.

Other embodiments concern use of compounds discussed herein to treat inflammatory disorders or diseases. Inflammatory disorders and diseases include, but are not limited to, acne vulgaris, arthritis, asthma, atherosclerosis, celiac disease, chronic prostatitis, colitis, Chron's disease, dermatitis, hepatitis, inflammatory bowel disease, interstitial cystis, nephritis, pelvic inflammatory disease, rheumatoid arthritis, ulcerative colitis, and vasculitis. Additionally, autoimmune disorders may be associated with elevated copper levels (Sorenson 1998). Exemplary autoimmune disorders include acute disseminated encephalomyelitis, Addison's disease, alopecia, autoimmune chronic active hepatitis, autoimmune hemolytic anemia, autoimmune pancreatitis, Behcet's syndrome, central nervous system vasculitis, Chron's disease, dermatitis herpetiformis, encephalomyelitis, Graves' disease, Gullain-Barre syndrome, Hashimoto's thyroiditis, hypersensitivity vasculitis, insulin-dependent diabetes mellitus, Isaacs' syndrome, Kawasaki disease, lupus, myasthenia gravis, multifocal motor neuropathy, neutropenia, polyarteritis nodosa, dermatomyositis, primary biliary cirrhosis, retinopathy, rheumatoid arthritis, systemic sclerosis, thyroiditis, and vasculitis.

Other copper-related disorders include fibrotic diseases including idiopathic pulmonary fibrosis, liver fibrosis, and primary biliary cirrhosis (Brewer 2003), and neurodegenerative diseases including alzheimer's disease, huntington's disease, amyotrophic lateral sclerosis, and multiple sclerosis (Rivera 2010).

In specific embodiments, a patient may have symptoms of, be at risk for, or have been diagnosed with a disorder or disease discussed herein.

Therefore, embodiments are contemplated to cover a number of methods involving an Atox1 inhibitor, which may decrease, inhibit or reduce Atox1 activity by or by at least 10, 15, 20, 25, 30, 35, 40, 45, 50, 55, 60, 65, 70, 75, 80, 85, 90, 95, or 100% (and any range derivable therein) compared to Atox1 activity in the absence of the Atox1 inhibitor. Therefore, in some embodiments, there are methods for inhibiting Atox1 in a cell comprising providing to the cell an effective amount of a small molecule that directly inhibits Atox1 activity in a cell.

Additionally, the embodiments are contemplated to cover a number of methods involving a CCS inhibitor, which may decrease, inhibit or reduce involving CCS activity by or by at least 10, 15, 20, 25, 30, 35, 40, 45, 50, 55, 60, 65, 70, 75, 80, 85, 90, 95, or 100% (and any range derivable therein) compared to CCS activity in the absence of the CCS inhibitor. Therefore, in some embodiments, there are methods for inhibiting CCS in a cell comprising providing to the cell an effective amount of a small molecule that directly inhibits CCS activity in a cell.

Single or multiple doses of the inhibitor(s) are contemplated. Desired time intervals for delivery of multiple doses can be determined by one of ordinary skill in the art employing no more than routine experimentation. As an example, subjects may be administered two doses daily at approximately 12 hour intervals. In some embodiments, the inhibitor(s) is administered once a day.

The inhibitor(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the inhibitor(s) may taken orally and that the timing of which is or is not dependent upon food intake. Thus, for example, the inhibitor can be taken every morning and/or every evening, regardless of when the subject has eaten or will eat.

Embodiments concern one or more of the following compounds, or derivatives, salts, or prodrugs thereof:

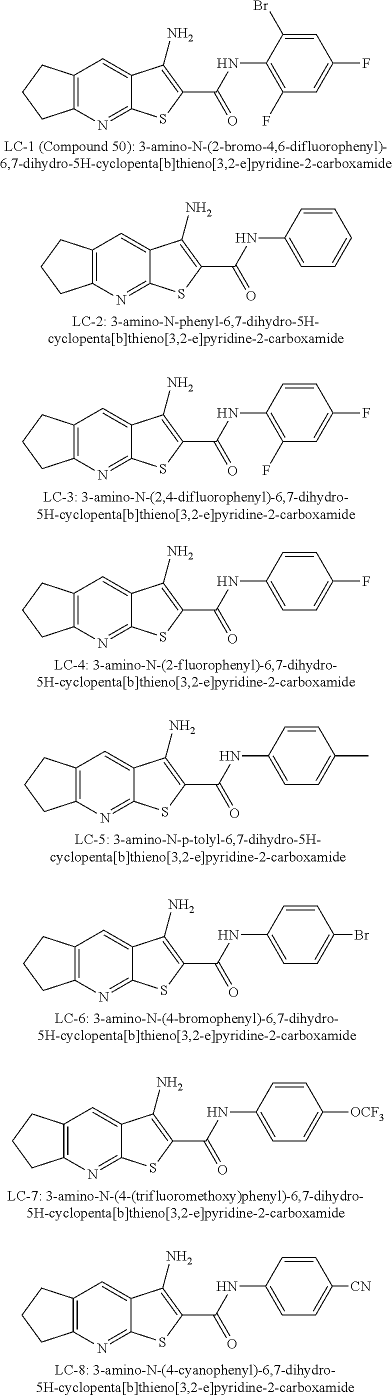

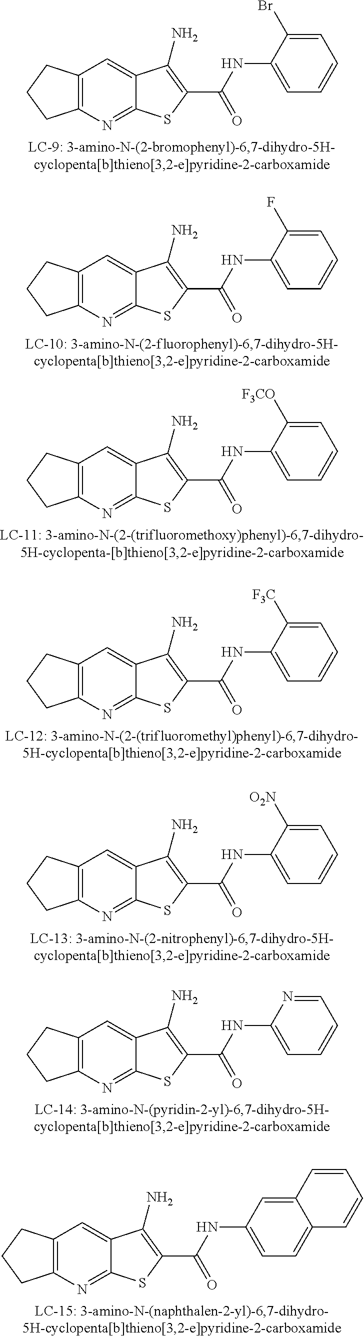

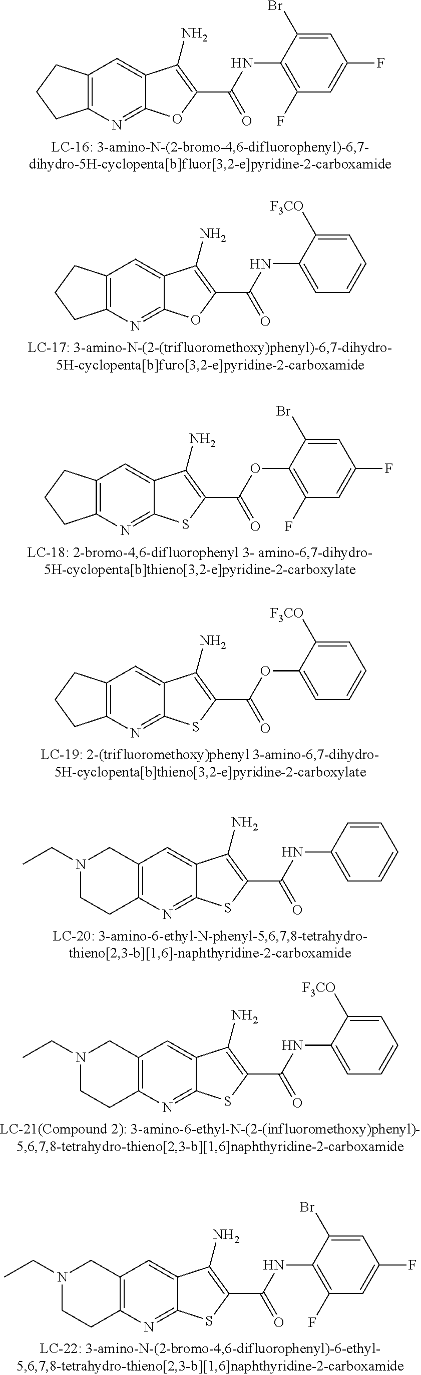

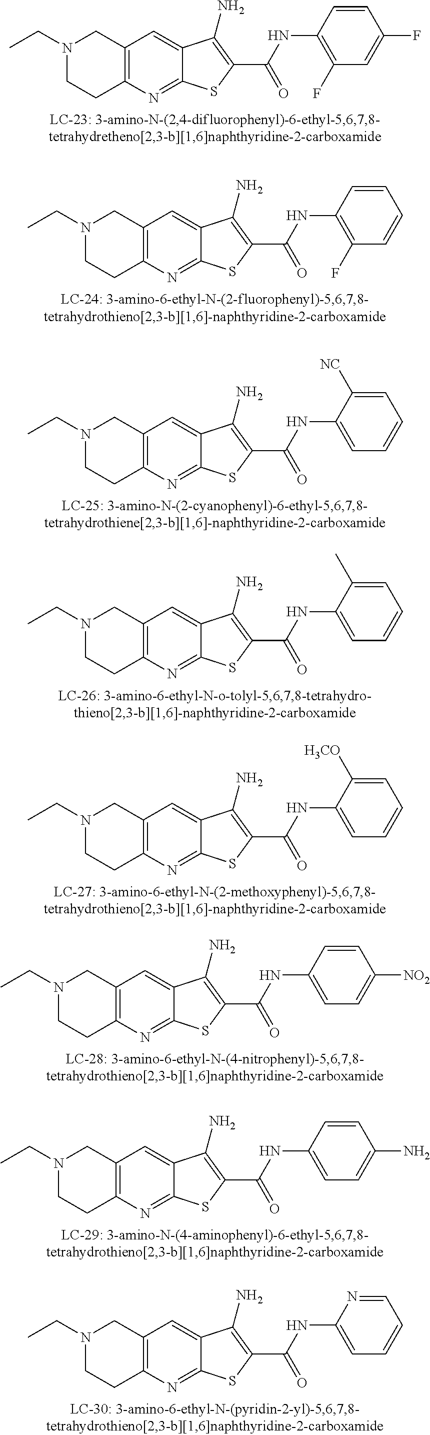

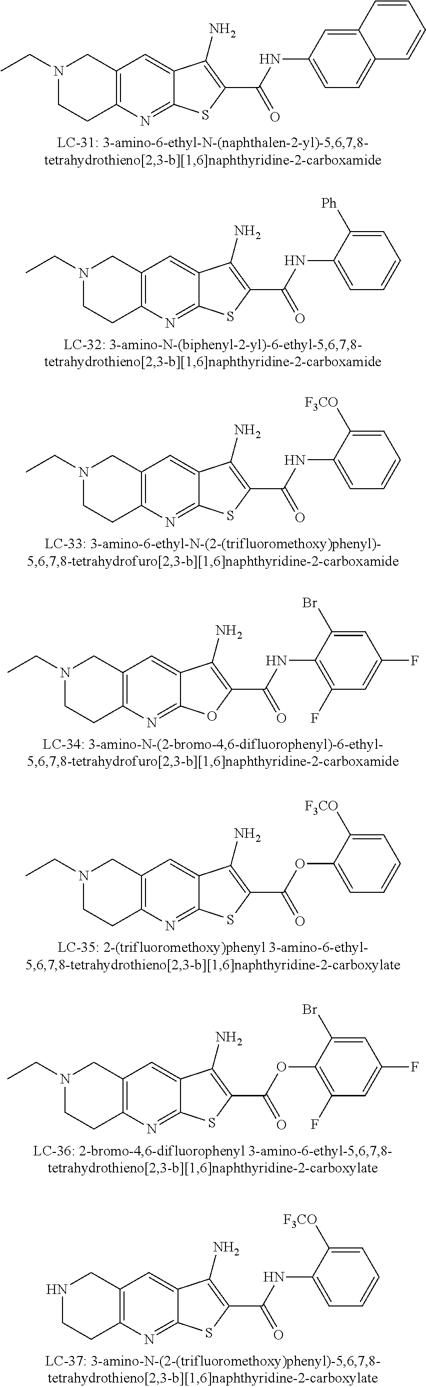

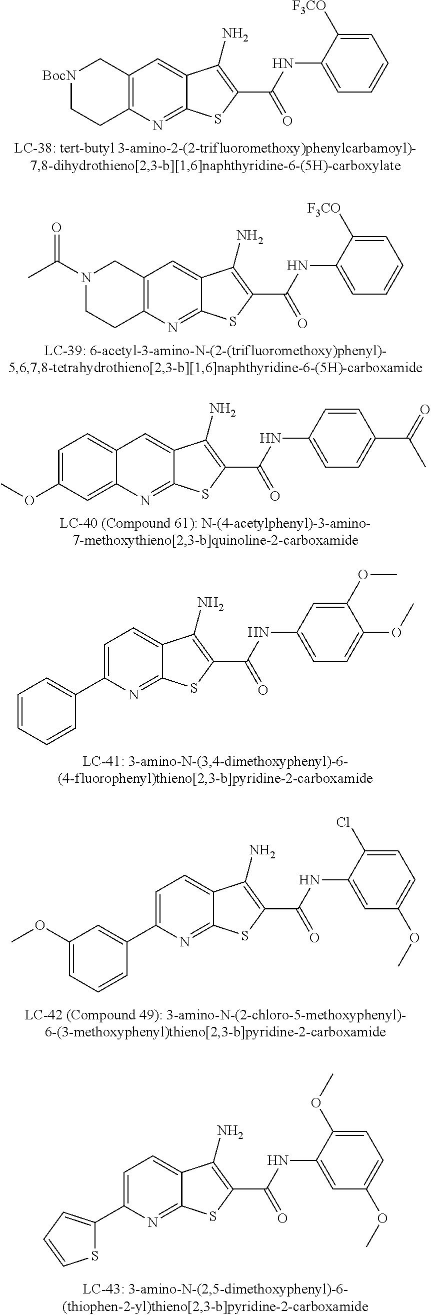

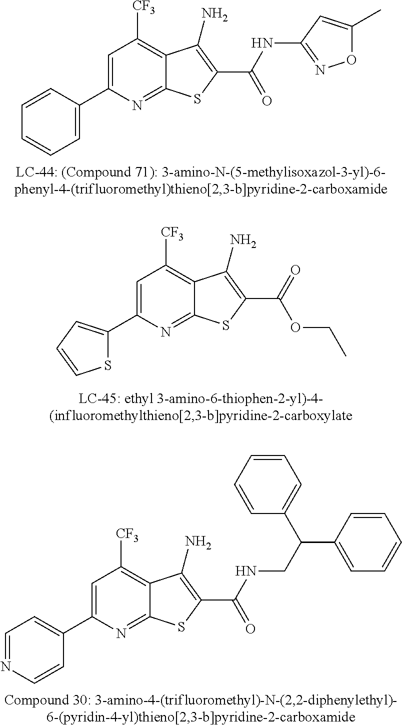 [SMILES: Cc1cc(NC(=O)c2sc3nc(-c4ccccc4)cc(C(F)(F)F)c3c2N)no1 ; CCOC(=O)c1sc2nc(-c3cccs3)cc(C(F)(F)F)c2c1N ; Nc1c(C(=O)NCC(c2ccccc2)c2ccccc2)sc2nc(-c3ccncc3)cc(C(F)(F)F)c12]

### CHEMICAL DEFINITIONS

As used herein, a “small molecule” refers to an organic compound that is either synthesized via conventional organic chemistry methods (e.g., in a laboratory) or found in nature. Typically, a small molecule is characterized in that it contains several carbon-carbon bonds, and has a molecular weight of less than about 1500 grams/mole. In certain embodiments, small molecules are less than about 1000 grams/mole. In certain embodiments, small molecules are less than about 550 grams/mole. In certain embodiments, small molecules are between about 200 and about 550 grams/mole. In certain embodiments, small molecules exclude peptides (e.g., compounds comprising 2 or more amino acids joined by a peptidyl bond). In certain embodiments, small molecules exclude nucleic acids.

As used herein, the term “amino” means —NH₂; the term “nitro” means —NO₂; the term “halo” or “halogen” designates —F, —Cl, —Br or —I; the term “mercapto” means —SH; the term “cyano” means —CN; the term “azido” means —N₃; the term “silyl” means —SiH₃, and the term “hydroxy” means —OH. In certain embodiments, a halogen may be —Br or —I.

As used herein, a “monovalent anion” refers to anions of a −1 charge. Such anions are well-known to those of skill in the art. Non-limiting examples of monovalent anions include halides (e.g., F⁻, Cl⁻, Br⁻ and I⁻), NO₂⁻, NO₃⁻, hydroxide (OH⁻) and azide (N₃⁻). As used herein, the structure [symbol] indicates that the bond may be a single bond or a double bond. Those of skill in the chemical arts understand that in certain circumstances, a double bond between two particular atoms is chemically feasible and in certain circumstances, a double bond is not. It is therefore contemplated that in some embodiments a double bond may be formed only when chemically feasible.

The term “alkyl” includes straight-chain alkyl, branched-chain alkyl, cycloalkyl (alicyclic), cyclic alkyl, heteroatom-unsubstituted alkyl, heteroatom-substituted alkyl, heteroatom-unsubstituted Cₙ-alkyl, and heteroatom-substituted Cₙ-alkyl. In certain embodiments, lower alkyls are contemplated. The term “lower alkyl” refers to alkyls of 1-6 carbon atoms (that is, 1, 2, 3, 4, 5 or 6 carbon atoms). The term “C₁-C₆ alkyl” refers to an alkyl group comprising 1, 6, or any intermediate integer value number of carbon atoms (that is, —C₁, —C₂, —C₃, —C₄, —C₅, or —C₆). The term “heteroatom-unsubstituted Cₙ-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, and cyclohexyl, are all non-limiting examples of heteroatom-unsubstituted alkyl groups. The term “heteroatom-substituted Cₙ-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The term “halogen-substituted C₁-C₆ alkyl” refers to an alkyl group comprising 1, 6, or any intermediate integer value number of carbon atoms (that is, —C₁, —C₂, —C₃, —C₄, —C₅, or —C₆), further comprising at least one halogen atom, for example, trifluoromethyl(—CF₃), —CH₂F, —CH₂Cl, —CH₂Br, etc, The following groups are all non-limiting examples of heteroatom-substituted alkyl groups: —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃. The term “C₅-C₇ cycloalkyl” refers to a closed ring comprising 5, 6, or 7 saturated carbon atoms. The term. “substituted C₁-C₆ alkyl” refers to an alkyl group comprising 1, 6, or any intermediate integer value number of carbon atoms (that is, —C₁, —C₂, —C₃, —C₄, —C₅, or —C₆) further comprising at least one substituent, for example, the substituent is phenyl.

The term “alkenyl” includes straight-chain alkenyl, branched-chain alkenyl, cycloalkenyl, cyclic alkenyl, heteroatom-unsubstituted alkenyl, heteroatom-substituted alkenyl, heteroatom-unsubstituted Cₙ-alkenyl, and heteroatom-substituted Cₙ-alkenyl. In certain embodiments, lower alkenyls are contemplated. The term “lower alkenyl” refers to alkenyls of 1-6 carbon atoms (that is, 1, 2, 3, 4, 5 or 6 carbon atoms). The term “heteroatom-unsubstituted Cₙ-alkenyl” refers to a radical, having a linear or branched, cyclic or acyclic structure, further having at least one nonaromatic carbon-carbon double bond, but no carbon-carbon triple bonds, a total of n carbon atoms, three or more hydrogen atoms, and no heteroatoms. For example, a heteroatom-unsubstituted C₂-C₁₀-alkenyl has 2 to 10 carbon atoms. Heteroatom-unsubstituted alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “heteroatom-substituted Cₙ-alkenyl” refers to a radical, having a single nonaromatic carbon atom as the point of attachment and at least one nonaromatic carbon-carbon double bond, but no carbon-carbon triple bonds, further having a linear or branched, cyclic or acyclic structure, further having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C₂-C₁₀-alkenyl has 2 to 10 carbon atoms. The groups, —CH═CHF, —CH═CHCl and —CH═CHBr, are non-limiting examples of heteroatom-substituted alkenyl groups.

The term “aryl” includes heteroatom-unsubstituted aryl, heteroatom-substituted aryl, heteroatom-unsubstituted Cₙ-aryl, heteroatom-substituted Cₙ-aryl, heteroaryl, heterocyclic aryl groups, carbocyclic aryl groups, biaryl groups, and single-valent radicals derived from polycyclic fused hydrocarbons (PAHs). The term “heteroatom-unsubstituted Cₙ-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C₆-C₁₀-aryl has 6 to 10 carbon atoms. Non-limiting examples of heteroatom-unsubstituted aryl groups include phenyl (Ph), methylphenyl, (dimethyl)phenyl, —C₆H₄CH₂CH₃, —C₆H₄CH₂CH₂CH₃, —C₆H₄CH(CH₃)₂, —C₆H₄CH(CH₂)₂, —C₆H₃(CH₃)CH₂CH₃, —C₆H₄CH═CH₂, —C₆H₄CH═CHCH₃, —C₆H₄C≡CH, —C₆H₄C≡CCH₃, naphthyl, and the radical derived from biphenyl. The term “C₆-C₁₀ aromatic” refers to an aryl group comprising 6, 10, or any intermediate integer value number of carbon atoms (that is, —C₆, —C₇, —C₈, —C₉, or —C₁₀), for example, phenyl, naphthyl, etc. The term “heteroatom-substituted Cₙ-aryl” refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C₁-C₁₀-heteroaryl has 1 to 10 carbon atoms. Non-limiting examples of substituted C₆-C₁₀ aromatic groups include the groups: —C₆H₄F, —C₆H₃F₂, —C₆H₂BrF₂, —C₆H₄CH₃, —C₆H₄Cl, —C₆H₄Br, —C₆H₄I, —C₆H₄—C₆H₅, —C₆H₃ (OCH₃)₂, —C₆H₃Cl(OCH₃), —C₆H₄OH, —C₆H₄OCH₃, —C₆H₄OCF₃, —C₆H₄OCH₂CH₃, —C₆H₄OC(O)CH₃, —C₆H₄NO₂, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, —C₆H₄CON(CH₃)₂, etc. In certain embodiments, heteroatom-substituted aryl groups are contemplated. In certain embodiments, heteroatom-unsubstituted aryl groups are contemplate. In certain embodiments, an aryl group may be mono-, di-, tri-, tetra- or penta-substituted with one or more heteroatom-containing substitutents.

The term “aralkyl” includes heteroatom-unsubstituted aralkyl, heteroatom-substituted aralkyl, heteroatom-unsubstituted Cₙ-aralkyl, heteroatom-substituted Cₙ-aralkyl, heteroaralkyl, and heterocyclic aralkyl groups. In certain embodiments, lower aralkyls are contemplated. The term “lower aralkyl” refers to aralkyls of 7-12 carbon atoms (that is, 7, 8, 9, 10, 11 or 12 carbon atoms). The term “heteroatom-unsubstituted Cₙ-aralkyl” refers to a radical, having a single saturated carbon atom as the point of attachment, further having a total of n carbon atoms, wherein at least 6 of the carbon atoms form an aromatic ring structure containing only carbon atoms, 7 or more hydrogen atoms, and no heteroatoms. For example, a heteroatom-unsubstituted C₇-C₁₀-aralkyl has 7 to 10 carbon atoms. Non-limiting examples of heteroatom-unsubstituted aralkyls are: phenylmethyl (benzyl, Bn) and phenylethyl. The term “heteroatom-substituted Cₙ-aralkyl” refers to a radical, having a single saturated carbon atom as the point of attachment, further having a total of n carbon atoms, 0, 1, or more than one hydrogen atom, and at least one heteroatom, wherein at least one of the carbon atoms is incorporated an aromatic ring structures, further wherein each heteroatom is independently selected from the group consisting of N, O, F, Cl, Br, I, Si, P, and S. For example, a heteroatom-substituted C₂-C₁₀-heteroaralkyl has 2 to 10 carbon atoms.

The term “acyl” includes straight-chain acyl, branched-chain acyl, cycloacyl, cyclic acyl, heteroatom-unsubstituted acyl, heteroatom-substituted acyl, heteroatom-unsubstituted Cₙ-acyl, heteroatom-substituted Cₙ-acyl, alkylcarbonyl, alkoxycarbonyl and aminocarbonyl groups. In certain embodiments, lower acyls are contemplated. The term “lower acyl” refers to acyls of 1-6 carbon atoms (that is, 1, 2, 3, 4, 5 or 6 carbon atoms). The term “C₂-C₆ acyl” refers to an acyl group comprising 1, 6 or any intermediate integer value number of carbon atoms, whereby the carbon atom that is the point of attachment is attached to a carbonyl group. The term “heteroatom-unsubstituted Cₙ-acyl” refers to a radical, having a single carbon atom of a carbonyl group as the point of attachment, further having a linear or branched, cyclic or acyclic structure, further having a total of n carbon atoms, 1 or more hydrogen atoms, a total of one oxygen atom, and no additional heteroatoms. For example, a heteroatom-unsubstituted C₁-C₁₀-acyl has 1 to 10 carbon atoms. The groups, —CHO, —C(O)CH₃, —C(O)CH₂CH₃, —C(O)CH₂CH₂CH₃, —C(O)CH(CH₃)₂, —C(O)CH(CH₂)₂, —C(O)C₆H₅, C(O)C₆H₄CH₃, —C(O)C₆H₄CH₂CH₃, and —COC₆H₃(CH₃)₂, are non-limiting examples of heteroatom-unsubstituted acyl groups. The term “heteroatom-substituted Cₙ-acyl” refers to a radical, having a single carbon atom as the point of attachment, the carbon atom being part of a carbonyl group, further having a linear or branched, cyclic or acyclic structure, further having a total of n carbon atoms, 0, 1, or more than one hydrogen atom, at least one additional heteroatom, in addition to the oxygen of the carbonyl group, wherein each additional heteroatom is independently selected from the group consisting of N, O, F, Cl, Br, I, Si, P, and S. For example, a heteroatom-substituted C₁-C₁₀-acyl has 1 to 10 carbon atoms. The groups, —C(O)CH₂CF₃, —CO₂H, —CO₂⁻, —CO₂CH₃, —CO₂CH₂CH₃, —CO₂CH₂CH₂CH₃, —CO₂CH(CH₃)₂, —CO₂CH(CH₂)₂, —C(O)NH₂ (carbamoyl), —C(O)NHCH₃, —C(O)NHCH₂CH₃, —CONHCH(CH₃)₂, —CONHCH(CH₂)₂, —CON(CH₃)₂, and —CONHCH₂CF₃, are non-limiting examples of heteroatom-substituted acyl groups.

The term “alkoxy” includes straight-chain alkoxy, branched-chain alkoxy, cycloalkoxy, cyclic alkoxy, heteroatom-unsubstituted alkoxy, heteroatom-substituted alkoxy, heteroatom-unsubstituted Cₙ-alkoxy, and heteroatom-substituted Cₙ-alkoxy. In certain embodiments, lower alkoxys are contemplated. The term “lower alkoxy” refers to alkoxys of 1-6 carbon atoms (that is, 1, 2, 3, 4, 5 or 6 carbon atoms). The term “C₁-C₆ alkoxy” refers to an alkyl group, comprising 1, 6 or any intermediate integer value number of carbon atoms, attached to an oxygen atom (that is, —OC₁, —OC₂, —OC₃, —OC₄, —OC₅, or —OC₆), whereby the oxygen atom is the point of attachment. The term “heteroatom-unsubstituted Cₙ-alkoxy” refers to a group, having the structure —OR, in which R is a heteroatom-unsubstituted Cₙ-alkyl, as that term is defined above. Heteroatom-unsubstituted alkoxy groups include: —OCH₃, —OCH₂CH₃, —OCH₂CH₂CH₃, —OCH(CH₃)₂, and —OCH(CH₂)₂. The term “heteroatom-substituted Cₙ-alkoxy” refers to a group, having the structure —OR, in which R is a heteroatom-substituted Cₙ-alkyl, as that term is defined above. For example, —OCH₂CF₃ is a heteroatom-substituted alkoxy group. The term “halogen-substituted C₁-C₆ alkoxy” refers to an alkyl group, comprising 1, 6, or any intermediate integer value number of carbon atoms, attached to an oxygen atom (that is, —OC₁, —OC₂, —OC₃, —OC₄, —OC₅, or —OC₆), whereby the oxygen atom is the point of attachment, further comprising at least one halogen atom, for example, —OCF₃, etc.

The term “alkenyloxy” includes straight-chain alkenyloxy, branched-chain alkenyloxy, cycloalkenyloxy, cyclic alkenyloxy, heteroatom-unsubstituted alkenyloxy, heteroatom-substituted alkenyloxy, heteroatom-unsubstituted Cₙ-alkenyloxy, and heteroatom-substituted Cₙ-alkenyloxy. The term “heteroatom-unsubstituted Cₙ-alkenyloxy” refers to a group, having the structure —OR, in which R is a heteroatom-unsubstituted Cₙ-alkenyl, as that term is defined above. The term “heteroatom-substituted Cₙ-alkenyloxy” refers to a group, having the structure —OR, in which R is a heteroatom-substituted Cₙ-alkenyl, as that term is defined above.

The term “alkynyloxy” includes straight-chain alkynyloxy, branched-chain alkynyloxy, cycloalkynyloxy, cyclic alkynyloxy, heteroatom-unsubstituted alkynyloxy, heteroatom-substituted alkynyloxy, heteroatom-unsubstituted Cₙ-alkynyloxy, and heteroatom-substituted Cₙ-alkynyloxy. The term “heteroatom-unsubstituted Cₙ-alkynyloxy” refers to a group, having the structure —OR, in which R is a heteroatom-unsubstituted Cₙ-alkynyl, as that term is defined above. The term “heteroatom-substituted Cₙ-alkynyloxy” refers to a group, having the structure —OR, in which R is a heteroatom-substituted Cₙ-alkynyl, as that term is defined above.

The term “aryloxy” includes heteroatom-unsubstituted aryloxy, heteroatom-substituted aryloxy, heteroatom-unsubstituted Cₙ-aryloxy, heteroatom-substituted Cₙ-aryloxy, heteroaryloxy, and heterocyclic aryloxy groups. The term “heteroatom-unsubstituted Cₙ-aryloxy” refers to a group, having the structure —OAr, in which Ar is a heteroatom-unsubstituted Cₙ-aryl, as that term is defined above. A non-limiting example of a heteroatom-unsubstituted aryloxy group is —OC₆H₅. The term “heteroatom-substituted Cₙ-aryloxy” refers to a group, having the structure —OAr, in which Ar is a heteroatom-substituted Cₙ-aryl, as that term is defined above.

The term “aralkyloxy” includes heteroatom-unsubstituted aralkyloxy, heteroatom-substituted aralkyloxy, heteroatom-unsubstituted Cₙ-aralkyloxy, heteroatom-substituted Cₙ-aralkyloxy, heteroaralkyloxy, and heterocyclic aralkyloxy groups. The term “heteroatom-unsubstituted Cₙ-aralkyloxy” refers to a group, having the structure —OAr, in which Ar is a heteroatom-unsubstituted Cₙ-aralkyl, as that term is defined above. The term “heteroatom-substituted Cₙ-aralkyloxy” refers to a group, having the structure —OAr, in which Ar is a heteroatom-substituted Cₙ-aralkyl, as that term is defined above.

The term “acyloxy” includes straight-chain acyloxy, branched-chain acyloxy, cycloacyloxy, cyclic acyloxy, heteroatom-unsubstituted acyloxy, heteroatom-substituted acyloxy, heteroatom-unsubstituted Cₙ-acyloxy, heteroatom-substituted Cₙ-acyloxy, alkylcarbonyloxy, arylcarbonyloxy, alkoxycarbonyloxy, aryloxycarbonyloxy, and carboxylate groups. The term “heteroatom-unsubstituted Cₙ-acyloxy” refers to a group, having the structure —OAc, in which Ac is a heteroatom-unsubstituted Cₙ-acyl, as that term is defined above. For example, —OC(O)CH₃ is a non-limiting example of a heteroatom-unsubstituted acyloxy group. The term “heteroatom-substituted Cₙ-acyloxy” refers to a group, having the structure —OAc, in which Ac is a heteroatom-substituted Cₙ-acyl, as that term is defined above. For example, —OC(O)OCH₃ and —OC(O)NHCH₃ are non-limiting examples of heteroatom-unsubstituted acyloxy groups. The term “C₂-C₆ alkyl carboxylate” refers to an acyloxy group comprising 2, 6, or any intermediate integer value number of carbon atoms (that is —OC(O)C₁, —OC(O)C₂, —OC(O)C₃, —OC(O)C₄, —OC(O)C₅).

The term “C₃-C₁₅ heterocyclic group” refers to a cyclic, bicyclic, or tricyclic group comprising 3, 15 or any intermediate integer value number of carbon atoms, of which at least one atom is not a carbon atom. Non-limiting examples of C₃-C₁₅ heterocyclic groups include the groups: furanyl, thienyl, isoxazole, piperidine, pyridyl, pyrrolyl, pyrimidyl, pyrazinyl, quinolyl, indolyl, and imidazoyl, wherein the substituent on C₃-C₁₅ heterocyclic group is 1 to 3 substituent(s) selected from halo, C₁-C₆ alkyl, halogen-substituted C₁-C₆ alkyl, C₁-C₆ alkoxy, halogen-substituted C₁-C₆ alkoxy, cyano, nitro, hydroxyl, amino, C₁-C₆ acyl, or C₁-C₆ alkyl carboxylate(—CO₂—C₁-C₆ alkyl), phenyl, —OC(O)C₁-C₆ alkyl, C₁-C₆ alkyl-substituted amino, —C₁-C₆ alkyl-OH, —C₁-C₆ alkyl-NH₂, aldehyde, —C(O)C₆H₅, carboxyl, amide, C₁-C₆ alkyl-substituted amide. Preferred, the substituent on C₃-C₁₅ heterocyclic group is 1 to 3 substituent(s) selected from —F, —Br, —CH₃, —CH₂CH₃, —Cl, —I, —C₆H₅, —OCH₃, —OH, —OCF₃, —OCH₂CH₃, —OC(O)CH₃, —NO₂, —NH₂, —NHCH₃, —N(CH₃)₂, —CH₂OH, —CH₂NH₂, —CF₃, —CN, —CHO, —C(O)CH₃, —C(O)C₆H₅, —CO₂H, —CO₂CH₃, —CO₂Bu-t, —CONH₂, —CONHCH₃, —CON(CH₃)₂, etc.

The term “C₈-C₁₅ condensed ring” refers to a cyclic, bicyclic, or tricyclic group comprising 8, 15 or any intermediate integer value number of carbon atoms.

The term “alkylamino” includes straight-chain alkylamino, branched-chain alkylamino, cycloalkylamino, cyclic alkylamino, heteroatom-unsubstituted alkylamino, heteroatom-substituted alkylamino, heteroatom-unsubstituted Cₙ-alkylamino, and heteroatom-substituted Cₙ-alkylamino. The term “heteroatom-unsubstituted Cₙ-alkylamino” refers to a radical, having a single nitrogen atom as the point of attachment, further having one or two saturated carbon atoms attached to the nitrogen atom, further having a linear or branched, cyclic or acyclic structure, containing a total of n carbon atoms, all of which are nonaromatic, 4 or more hydrogen atoms, a total of 1 nitrogen atom, and no additional heteroatoms. For example, a heteroatom-unsubstituted C₁-C₁₀-alkylamino has 1 to 10 carbon atoms. The term “heteroatom-unsubstituted Cₙ-alkylamino” includes groups, having the structure —NHR, in which R is a heteroatom-unsubstituted Cₙ-alkyl, as that term is defined above. A heteroatom-unsubstituted alkylamino group would include —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —N(CH₃)₂, —N(CH₃)CH₂CH₃, —N(CH₂CH₃)₂, N-pyrrolidinyl, and N-piperidinyl. The term “heteroatom-substituted Cₙ-alkylamino” refers to a radical, having a single nitrogen atom as the point of attachment, further having one or two saturated carbon atoms attached to the nitrogen atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₁-C₁₀-alkylamino has 1 to 10 carbon atoms. The term “heteroatom-substituted Cₙ-alkylamino” includes groups, having the structure —NHR, in which R is a heteroatom-substituted Cₙ-alkyl, as that term is defined above.

The term “alkenylamino” includes straight-chain alkenylamino, branched-chain alkenylamino, cycloalkenylamino, cyclic alkenylamino, heteroatom-unsubstituted alkenylamino, heteroatom-substituted alkenylamino, heteroatom-unsubstituted Cₙ-alkenylamino, heteroatom-substituted Cₙ-alkenylamino, dialkenylamino, and alkyl(alkenyl)amino groups. The term “heteroatom-unsubstituted Cₙ-alkenylamino” refers to a radical, having a single nitrogen atom as the point of attachment, further having one or two carbon atoms attached to the nitrogen atom, further having a linear or branched, cyclic or acyclic structure, containing at least one nonaromatic carbon-carbon double bond, a total of n carbon atoms, 4 or more hydrogen atoms, a total of one nitrogen atom, and no additional heteroatoms. For example, a heteroatom-unsubstituted C₂-C₁₀-alkenylamino has 2 to 10 carbon atoms. The term “heteroatom-unsubstituted Cₙ-alkenylamino” includes groups, having the structure —NHR, in which R is a heteroatom-unsubstituted Cₙ-alkenyl, as that term is defined above. The term “heteroatom-substituted Cₙ-alkenylamino” refers to a radical, having a single nitrogen atom as the point of attachment and at least one nonaromatic carbon-carbon double bond, but no carbon-carbon triple bonds, further having one or two carbon atoms attached to the nitrogen atom, further having a linear or branched, cyclic or acyclic structure, further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkenylamino has 2 to 10 carbon atoms. The term “heteroatom-substituted Cₙ-alkenylamino” includes groups, having the structure —NHR, in which R is a heteroatom-substituted Cₙ-alkenyl, as that term is defined above.

The term “alkynylamino” includes straight-chain alkynylamino, branched-chain alkynylamino, cycloalkynylamino, cyclic alkynylamino, heteroatom-unsubstituted alkynylamino, heteroatom-substituted alkynylamino, heteroatom-unsubstituted Cₙ-alkynylamino, heteroatom-substituted Cₙ-alkynylamino, dialkynylamino, alkyl(alkynyl)amino, and alkenyl(alkynyl)amino groups. The term “heteroatom-unsubstituted Cₙ-alkynylamino” refers to a radical, having a single nitrogen atom as the point of attachment, further having one or two carbon atoms attached to the nitrogen atom, further having a linear or branched, cyclic or acyclic structure, containing at least one carbon-carbon triple bond, a total of n carbon atoms, at least one hydrogen atoms, a total of one nitrogen atom, and no additional heteroatoms. For example, a heteroatom-unsubstituted C₂-C₁₀-alkynylamino has 2 to 10 carbon atoms. The term “heteroatom-unsubstituted Cₙ-alkynylamino” includes groups, having the structure —NHR, in which R is a heteroatom-unsubstituted Cₙ-alkynyl, as that term is defined above. The term “heteroatom-substituted Cₙ-alkynylamino” refers to a radical, having a single nitrogen atom as the point of attachment, further having one or two carbon atoms attached to the nitrogen atom, further having at least one nonaromatic carbon-carbon triple bond, further having a linear or branched, cyclic or acyclic structure, and further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkynylamino has 2 to 10 carbon atoms. The term “heteroatom-substituted Cₙ-alkynylamino” includes groups, having the structure —NHR, in which R is a heteroatom-substituted Cₙ-alkynyl, as that term is defined above.

The term “arylamino” includes heteroatom-unsubstituted arylamino, heteroatom-substituted arylamino, heteroatom-unsubstituted Cₙ-arylamino, heteroatom-substituted Cₙ-arylamino, heteroarylamino, heterocyclic arylamino, and alkyl(aryl)amino groups. The term “heteroatom-unsubstituted Cₙ-arylamino” refers to a radical, having a single nitrogen atom as the point of attachment, further having at least one aromatic ring structure attached to the nitrogen atom, wherein the aromatic ring structure contains only carbon atoms, further having a total of n carbon atoms, 6 or more hydrogen atoms, a total of one nitrogen atom, and no additional heteroatoms. For example, a heteroatom-unsubstituted C₆-C₁₀-arylamino has 6 to 10 carbon atoms. The term “heteroatom-unsubstituted Cₙ-arylamino” includes groups, having the structure —NHR, in which R is a heteroatom-unsubstituted Cₙ-aryl, as that term is defined above. The term “heteroatom-substituted Cₙ-arylamino” refers to a radical, having a single nitrogen atom as the point of attachment, further having a total of n carbon atoms, at least one hydrogen atom, at least one additional heteroatoms, that is, in addition to the nitrogen atom at the point of attachment, wherein at least one of the carbon atoms is incorporated into one or more aromatic ring structures, further wherein each additional heteroatom is independently selected from the group consisting of N, O, F, Cl, Br, I, Si, P, and S. For example, a heteroatom-substituted C₆-C₁₀-arylamino has 6 to 10 carbon atoms. The term “heteroatom-substituted Cₙ-arylamino” includes groups, having the structure —NHR, in which R is a heteroatom-substituted Cₙ-aryl, as that term is defined above.

The term “aralkylamino” includes heteroatom-unsubstituted aralkylamino, heteroatom-substituted aralkylamino, heteroatom-unsubstituted Cₙ-aralkylamino, heteroatom-substituted Cₙ-aralkylamino, heteroaralkylamino, heterocyclic aralkylamino groups, and diaralkylamino groups. The term “heteroatom-unsubstituted Cₙ-aralkylamino” refers to a radical, having a single nitrogen atom as the point of attachment, further having one or two saturated carbon atoms attached to the nitrogen atom, further having a total of n carbon atoms, wherein at least 6 of the carbon atoms form an aromatic ring structure containing only carbon atoms, 8 or more hydrogen atoms, a total of one nitrogen atom, and no additional heteroatoms. For example, a heteroatom-unsubstituted C₇-C₁₀-aralkylamino has 7 to 10 carbon atoms. The term “heteroatom-unsubstituted Cₙ-aralkylamino” includes groups, having the structure —NHR, in which R is a heteroatom-unsubstituted Cₙ-aralkyl, as that term is defined above. The term “heteroatom-substituted Cₙ-aralkylamino” refers to a radical, having a single nitrogen atom as the point of attachment, further having at least one or two saturated carbon atoms attached to the nitrogen atom, further having a total of n carbon atoms, 0, 1, or more than one hydrogen atom, at least one additional heteroatom, that is, in addition to the nitrogen atom at the point of attachment, wherein at least one of the carbon atom incorporated into an aromatic ring, further wherein each heteroatom is independently selected from the group consisting of N, O, F, Cl, Br, I, Si, P, and S. For example, a heteroatom-substituted C₇-C₁₀-aralkylamino has 7 to 10 carbon atoms. The term “heteroatom-substituted Cₙ-aralkylamino” includes groups, having the structure —NHR, in which R is a heteroatom-substituted Cₙ-aralkyl, as that term is defined above.

The term “amido” includes straight-chain amido, branched-chain amido, cycloamido, cyclic amido, heteroatom-unsubstituted amido, heteroatom-substituted amido, heteroatom-unsubstituted Cₙ-amido, heteroatom-substituted Cₙ-amido, alkylcarbonylamino, arylcarbonylamino, alkoxycarbonylamino, aryloxycarbonylamino, acylamino, alkylaminocarbonylamino, arylaminocarbonylamino, and ureido groups. The term “heteroatom-unsubstituted Cₙ-amido” refers to a radical, having a single nitrogen atom as the point of attachment, further having a carbonyl group attached via its carbon atom to the nitrogen atom, further having a linear or branched, cyclic or acyclic structure, further having a total of n carbon atoms, 1 or more hydrogen atoms, a total of one oxygen atom, a total of one nitrogen atom, and no additional heteroatoms. For example, a heteroatom-unsubstituted C₁-C₁₀-amido has 1 to 10 carbon atoms. The term “heteroatom-unsubstituted Cₙ-amido” includes groups, having the structure —NHR, in which R is a heteroatom-unsubstituted Cₙ-acyl, as that term is defined above. The group, —NHC(O)CH₃, is a non-limiting example of a heteroatom-unsubstituted amido group. The term “heteroatom-substituted Cₙ-amido” refers to a radical, having a single nitrogen atom as the point of attachment, further having a carbonyl group attached via its carbon atom to the nitrogen atom, further having a linear or branched, cyclic or acyclic structure, further having a total of n aromatic or nonaromatic carbon atoms, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. For example, a heteroatom-substituted C₁-C₁₀-amido has 1 to 10 carbon atoms. The term “heteroatom-substituted Cₙ-amido” includes groups, having the structure —NHR, in which R is a heteroatom-unsubstituted Cₙ-acyl, as that term is defined above. The group, —NHCO₂CH₃, is a non-limiting example of a heteroatom-substituted amido group.

The term “alkylthio” includes straight-chain alkylthio, branched-chain alkylthio, cycloalkylthio, cyclic alkylthio, heteroatom-unsubstituted alkylthio, heteroatom-substituted alkylthio, heteroatom-unsubstituted Cₙ-alkylthio, and heteroatom-substituted Cₙ-alkylthio. The term “heteroatom-unsubstituted Cₙ-alkylthio” refers to a group, having the structure —SR, in which R is a heteroatom-unsubstituted Cₙ-alkyl, as that term is defined above. The group, —SCH₃, is an example of a heteroatom-unsubstituted alkylthio group. The term “heteroatom-substituted Cₙ-alkylthio” refers to a group, having the structure —SR, in which R is a heteroatom-substituted Cₙ-alkyl, as that term is defined above.

The term “alkenylthio” includes straight-chain alkenylthio, branched-chain alkenylthio, cycloalkenylthio, cyclic alkenylthio, heteroatom-unsubstituted alkenylthio, heteroatom-substituted alkenylthio, heteroatom-unsubstituted Cₙ-alkenylthio, and heteroatom-substituted Cₙ-alkenylthio. The term “heteroatom-unsubstituted Cₙ-alkenylthio” refers to a group, having the structure —SR, in which R is a heteroatom-unsubstituted Cₙ-alkenyl, as that term is defined above. The term “heteroatom-substituted Cₙ-alkenylthio” refers to a group, having the structure —SR, in which R is a heteroatom-substituted Cₙ-alkenyl, as that term is defined above.

The term “alkynylthio” includes straight-chain alkynylthio, branched-chain alkynylthio, cycloalkynylthio, cyclic alkynylthio, heteroatom-unsubstituted alkynylthio, heteroatom-substituted alkynylthio, heteroatom-unsubstituted Cₙ-alkynylthio, and heteroatom-substituted Cₙ-alkynylthio. The term “heteroatom-unsubstituted Cₙ-alkynylthio” refers to a group, having the structure —SR, in which R is a heteroatom-unsubstituted Cₙ-alkynyl, as that term is defined above. The term “heteroatom-substituted Cₙ-alkynylthio” refers to a group, having the structure —SR, in which R is a heteroatom-substituted Cₙ-alkynyl, as that term is defined above.

The term “arylthio” includes heteroatom-unsubstituted arylthio, heteroatom-substituted arylthio, heteroatom-unsubstituted Cₙ-arylthio, heteroatom-substituted Cₙ-arylthio, heteroarylthio, and heterocyclic arylthio groups. The term “heteroatom-unsubstituted Cₙ-arylthio” refers to a group, having the structure —SAr, in which Ar is a heteroatom-unsubstituted Cₙ-aryl, as that term is defined above. The group, —SC₆H₅, is an example of a heteroatom-unsubstituted arylthio group. The term “heteroatom-substituted Cₙ-arylthio” refers to a group, having the structure —SAr, in which Ar is a heteroatom-substituted Cₙ-aryl, as that term is defined above.

The term “aralkylthio” includes heteroatom-unsubstituted aralkylthio, heteroatom-substituted aralkylthio, heteroatom-unsubstituted Cₙ-aralkylthio, heteroatom-substituted Cₙ-aralkylthio, heteroaralkylthio, and heterocyclic aralkylthio groups. The term “heteroatom-unsubstituted Cₙ-aralkylthio” refers to a group, having the structure —SAr, in which Ar is a heteroatom-unsubstituted Cₙ-aralkyl, as that term is defined above. The group, —SCH₂C₆H₅, is an example of a heteroatom-unsubstituted aralkyl group. The term “heteroatom-substituted Cₙ-aralkylthio” refers to a group, having the structure —SAr, in which Ar is a heteroatom-substituted Cₙ-aralkyl, as that term is defined above.

The term “acylthio” includes straight-chain acylthio, branched-chain acylthio, cycloacylthio, cyclic acylthio, heteroatom-unsubstituted acylthio, heteroatom-substituted acylthio, heteroatom-unsubstituted Cₙ-acylthio, heteroatom-substituted Cₙ-acylthio, alkylcarbonyloxy, arylcarbonyloxy, alkoxycarbonyloxy, aryloxycarbonyloxy, and carboxylate groups. The term “heteroatom-unsubstituted Cₙ-acylthio” refers to a group, having the structure —SAc, in which Ac is a heteroatom-unsubstituted Cₙ-acyl, as that term is defined above. The group, —SCOCH₃, is an example of a heteroatom-unsubstituted acylthio group. The term “heteroatom-substituted Cₙ-acylthio” refers to a group, having the structure —SAc, in which Ac is a heteroatom-substituted Cₙ-acyl, as that term is defined above.

The term “alkylsilyl” includes straight-chain alkylsilyl, branched-chain alkylsilyl, cycloalkylsilyl, cyclic alkylsilyl, heteroatom-unsubstituted alkylsilyl, heteroatom-substituted alkylsilyl, heteroatom-unsubstituted Cₙ-alkylsilyl, and heteroatom-substituted Cₙ-alkylsilyl. The term “heteroatom-unsubstituted Cₙ-alkylsilyl” refers to a radical, having a single silicon atom as the point of attachment, further having one, two, or three saturated carbon atoms attached to the silicon atom, further having a linear or branched, cyclic or acyclic structure, containing a total of n carbon atoms, all of which are nonaromatic, 5 or more hydrogen atoms, a total of 1 silicon atom, and no additional heteroatoms. For example, a heteroatom-unsubstituted C₁-C₁₀-alkylsilyl has 1 to 10 carbon atoms. An alkylsilyl group includes dialkylamino groups. The groups, —Si(CH₃)₃ and —Si(CH₃)₂C(CH₃)₃, are non-limiting examples of heteroatom-unsubstituted alkylsilyl groups. The term “heteroatom-substituted Cₙ-alkylsilyl” refers to a radical, having a single silicon atom as the point of attachment, further having at least one, two, or three saturated carbon atoms attached to the silicon atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the silicon atom at the point of attachment, wherein each additional heteroatom is independently selected from the group consisting of N, O, F, Cl, Br, I, Si, P, and S. For example, a heteroatom-substituted C₁-C₁₀-alkylsilyl has 1 to 10 carbon atoms.

The term “phosphonate” includes straight-chain phosphonate, branched-chain phosphonate, cyclophosphonate, cyclic phosphonate, heteroatom-unsubstituted phosphonate, heteroatom-substituted phosphonate, heteroatom-unsubstituted Cₙ-phosphonate, and heteroatom-substituted Cₙ-phosphonate. The term “heteroatom-unsubstituted Cₙ-phosphonate” refers to a radical, having a single phosphorous atom as the point of attachment, further having a linear or branched, cyclic or acyclic structure, further having a total of n carbon atoms, 2 or more hydrogen atoms, a total of three oxygen atom, and no additional heteroatoms. The three oxygen atoms are directly attached to the phosphorous atom, with one of these oxygen atoms doubly bonded to the phosphorous atom. For example, a heteroatom-unsubstituted C₀-C₁₀-phosphonate has 0 to 10 carbon atoms. The groups, —P(O)(OH)₂, —P(O)(OH)OCH₃, —P(O)(OH)OCH₂CH₃, —P(O)(OCH₃)₂, and —P(O)(OH)(OC₆H₅) are non-limiting examples of heteroatom-unsubstituted phosphonate groups. The term “heteroatom-substituted Cₙ-phosphonate” refers to a radical, having a single phosphorous atom as the point of attachment, further having a linear or branched, cyclic or acyclic structure, further having a total of n carbon atoms, 2 or more hydrogen atoms, three or more oxygen atoms, three of which are directly attached to the phosphorous atom, with one of these three oxygen atoms doubly bonded to the phosphorous atom, and further having at least one additional heteroatom in addition to the three oxygen atoms, wherein each additional heteroatom is independently selected from the group consisting of N, O, F, Cl, Br, I, Si, P, and S. For example, a heteroatom-unsubstituted C₀-C₁₀-phosphonate has 0 to 10 carbon atoms.

The term “phosphinate” includes straight-chain phosphinate, branched-chain phosphinate, cyclophosphinate, cyclic phosphinate, heteroatom-unsubstituted phosphinate, heteroatom-substituted phosphinate, heteroatom-unsubstituted Cₙ-phosphinate, and heteroatom-substituted Cₙ-phosphinate. The term “heteroatom-unsubstituted Cₙ-phosphinate” refers to a radical, having a single phosphorous atom as the point of attachment, further having a linear or branched, cyclic or acyclic structure, further having a total of n carbon atoms, 2 or more hydrogen atoms, a total of two oxygen atom, and no additional heteroatoms. The two oxygen atoms are directly attached to the phosphorous atom, with one of these oxygen atoms doubly bonded to the phosphorous atom. For example, a heteroatom-unsubstituted C₀-C₁₀-phosphinate has 0 to 10 carbon atoms. The groups, —P(O)(OH)H, —P(O)(OH)CH₃, —P(O)(OH)CH₂CH₃, —P(O)(OCH₃)CH₃, and —P(O)(OC₆H₅)H are non-limiting examples of heteroatom-unsubstituted phosphinate groups. The term “heteroatom-substituted Cₙ-phosphinate” refers to a radical, having a single phosphorous atom as the point of attachment, further having a linear or branched, cyclic or acyclic structure, further having a total of n carbon atoms, 2 or more hydrogen atoms, two or more oxygen atoms, two of which are directly attached to the phosphorous atom, with one of these two oxygen atoms doubly bonded to the phosphorous atom, and further having at least one additional heteroatom in addition to the two oxygen atoms, wherein each additional heteroatom is independently selected from the group consisting of N, O, F, Cl, Br, I, Si, P, and S. For example, a heteroatom-unsubstituted C₀-C₁₀-phosphinate has 0 to 10 carbon atoms.

Any apparently unfulfilled valency is to be understood to be properly filled by hydrogen atom(s). For example, a compound with a substituent of —O or —N is to be understood to be —OH or —NH₂, respectively.

Any genus, subgenus, or specific compound discussed herein is specifically contemplated as being excluded from any embodiment described herein.

Compounds described herein may be prepared synthetically using conventional organic chemistry methods known to those of skill in the art and/or are commercially available (e.g., ChemBridge Co., San Diego, Calif.).

Embodiments are also intended to encompass salts of any of the compounds provided herein. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like. A salt may be a pharmaceutically acceptable salt, for example. Thus, pharmaceutically acceptable salts of compounds are contemplated. In particular embodiments, the compound may be in the form of an ammonium salt.

The term “pharmaceutically acceptable salts,” as used herein, refers to salts of compounds disclosed herein that are substantially non-toxic to living organisms. Typical pharmaceutically acceptable salts include those salts prepared by reaction of a compound with an inorganic or organic acid, or an organic base, depending on the substituents present on the compounds.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like.

Suitable pharmaceutically acceptable salts may also be formed by reacting the agents with an organic base such as methylamine, ethylamine, ethanolamine, lysine, ornithine and the like.

Pharmaceutically acceptable salts include the salts formed between carboxylate or sulfonate groups found on some of the compounds and inorganic cations, such as sodium, potassium, ammonium, or calcium, or such organic cations as isopropylammonium, trimethylammonium, tetramethylammonium, and imidazolium.

Derivatives of compounds are also contemplated. In certain aspects, “derivative” refers to a chemically modified compound that still retains the desired effects of the compound prior to the chemical modification. Such derivatives may have the addition, removal, or substitution of one or more chemical moieties on the parent molecule. Non-limiting examples of the types modifications that can be made to the compounds and structures disclosed herein include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, heteroatoms such as N, S, or O can be substituted into the structure instead of a carbon atom.

Compounds employed in methods disclosed herein may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S- or the R-configuration, as defined by the IUPAC 1974 Recommendations. Compounds may be of the D- or L-form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers.

In addition, atoms making up the compounds are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

As noted above, compounds may exist or be administered in prodrug form. As used herein, “prodrug” is intended to include any covalently bonded carriers which release the active parent drug or compounds that are metabolized in vivo to an active drug or other compounds employed in methods in vivo when such a prodrug is administered to a subject. Since prodrugs are known to enhance numerous desirable qualities of pharmaceuticals (e.g., solubility, bioavailability, manufacturing, etc.), the compounds employed in some methods may, if desired, be delivered in prodrug form. Thus, prodrugs of compounds are contemplated as well as methods of delivering prodrugs. Prodrugs of the compounds employed in various embodiments may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a subject, cleaves to form a free hydroxyl, free amino, or carboxylic acid, respectively. Other examples include, but are not limited to, acetate, formate, and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, Selection and Use (2002), which is incorporated herein by reference.

### Pharmaceutical Formulations and Administration Thereof

Pharmaceutical compositions may comprise an effective amount of one or more candidate substance or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one candidate substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The compounds disclosed herein may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, systemically, subcutaneously, subconjunctival, intravesicularlly, mucosally, intrapericardially, intraumbilically, intraocularally, orally, locally, via inhalation (e.g., aerosol inhalation), via injection, via infusion, via continuous infusion, via localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 1990).

The actual dosage amount of a composition administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of a compound described herein. In other embodiments, the compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain other embodiments, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or μg/kg or micrograms/kg/minute or mg/kg/min or micrograms/kg/hour or mg/kg/hour, or any range derivable therein. Alternatively, a composition may contain about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 micrograms (μg), milligrams (mg) or micrograms (mcg) or M (molar), or any range derivable therein.

Compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

The candidate substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine, or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. It may be preferable to include isotonic agents, such as, for example, sugars, sodium chloride or combinations thereof.

In other embodiments, one may use eye drops, nasal solutions or sprays, aerosols or inhalants. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in certain embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the candidate substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. In certain embodiments, carriers for oral administration comprise inert diluents, assimilable edible carriers or combinations thereof. In other aspects, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar, or both.

Additional formulations which are suitable for other modes of administration include suppositories. Suppositories are solid dosage forms of various weights and shapes, usually medicated, for insertion into the rectum, vagina, or urethra. After insertion, suppositories soften, melt or dissolve in the cavity fluids. In general, for suppositories, traditional carriers may include, for example, polyalkylene glycols, triglycerides, or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, certain methods of preparation may include vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin, or combinations thereof.

In some embodiments, methods and compositions may be used to treat cancer. A cancer, for example, may be a recurrent cancer or a cancer that is known or suspected to be resistant to conventional therapeutic regimens and standard therapies.

Moreover, one or more compounds described herein can be used to prevent cancer or to treat pre-cancers or premalignant cells, including metaplasias, dysplasias, and hyperplasias. It may also be used to inhibit undesirable but benign cells, such as squamous metaplasia, dysplasia, benign prostate hyperplasia cells, hyperplastic lesions, and the like.

“Treatment” and “treating” refer to administration or application of an agent, drug, compositions, or remedy to a subject, or performance of a procedure or therapeutic action on a subject for the purpose of obtaining a therapeutic benefit against a disease or health-related condition. The term “therapeutic benefit” refers to anything that promotes or enhances the well-being of the subject with respect to the medical treatment of a condition, which includes, but is not limited to, treatment of pre-cancer, dysplasia, cancer, and other hyperproliferative diseases. A list of nonexhaustive examples of therapeutic benefit includes extension of the subject's life by any period of time, decrease or delay in the neoplastic development of the disease, decrease in hyperproliferation, reduction in tumor growth, delay of metastases or reduction in number of metastases, reduction in cancer cell number or tumor cell proliferation rate, decrease or delay in progression of neoplastic development from a premalignant condition, and a decrease in pain to the subject that can be attributed to the subject's condition.

A patient can be any animal, including a human, having, suspected of having, or at risk or heightened risk of having cancer and undergoes treatment for such. In many embodiments, a patient is a mammal, specifically a human. The patient/subject can be one known or suspected of being free of a particular disease or health-related condition at the time the inventive compositions and/or methods are administered. The subject, for example, can be a subject with no known disease or health-related condition (i.e., a healthy subject). In some embodiments, the subject is a subject at risk of developing a particular disease or health-related condition. For example, the subject or the subject's relatives may have a history of cancer, who is at risk of developing a cancer. Alternatively, the subject may have undergone failed cancer therapy. The subject may be a subject at risk of developing a recurrent cancer because of a genetic predisposition or as a result of past chemotherapy. Alternatively, the subject may be a subject with a history of successfully treated cancer who is currently disease-free, but who is at risk of developing a second primary tumor. For example, the risk may be the result of past radiation therapy or chemotherapy that was applied as treatment of a first primary tumor. In some embodiments, the subject may be a subject with a first disease or health-related condition, who is at risk of development of a second disease or health-related condition. In some embodiments, methods may involve identifying a patient in need of such treatment. A patient may be identified, for example, based on taking a patient history, having one or more tests done to determine that the patient has cancer or a tumor, operating on the patient or taking a biopsy.

Cancer cells that may be treated by methods and compositions described herein also include cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these (and it is contemplated that one or more of these may be excluded as part of an embodiment): neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

### Combination Therapy

In some embodiments, it is contemplated that the Atox1 and/or CCS inhibitors may be used in conjunction other therapies. This process may involve contacting the cell(s) with one or more of the inhibitors at the same time or within a period of time wherein separate administration of the inhibitors produces a desired therapeutic benefit. This may be achieved by contacting the cell, tissue or organism with a single composition or pharmacological formulation that includes two or more agents, or by contacting the cell with two or more distinct compositions or formulations, wherein one composition includes one agent and the other includes another.

The one or more compounds may precede, be co-current with and/or follow the other agents by intervals ranging from minutes to weeks. In embodiments where the agents are applied separately to a cell, tissue or organism, one would generally ensure that a significant period of time did not expire between the time of each delivery, such that the agents would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the candidate substance. In other aspects, one or more agents may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering the Atox1 and/or CCS inhibitor.

Various combination regimens of the agents may be employed. Non-limiting examples of such combinations are shown below, wherein an inhibitor is “A” and a second agent is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

In some embodiments, more than one course of therapy may be employed. It is contemplated that multiple courses may be implemented. In specific embodiments, a combination therapy is provided that combines at least one Atox1 and/or CCS inhibitor such as Compound 50 with at least one other cancer therapy. Cancer therapies that are contemplated include, but are not limited to, surgery, chemotherapy, radiation, or immunotherapy. It is also contemplated that more than one Atox1 and/or CCS inhibitor may be employed.

### Organisms and Cell Source

Cells that may be used in many embodiments can be from a variety of sources. Embodiments include the use of mammalian cells, such as cells from monkeys, chimpanzees, rabbits, mice, rats, ferrets, dogs, pigs, humans, and cows. Alternatively, the cells may be from fruit flies, yeast, or E. coli, which are all model systems for evaluating homologous recombination.

Embodiments can involve cells, tissues, or organs involving the heart, lung, kidney, liver, bone marrow, pancreas, skin, bone, vein, artery, cornea, blood, small intestine, large intestine, brain, spinal cord, smooth muscle, skeletal muscle, ovary, testis, uterus, and umbilical cord.

Moreover, methods can be employed in cells of the following type: platelet, myelocyte, erythrocyte, lymphocyte, adipocyte, fibroblast, epithelial cell, endothelial cell, smooth muscle cell, skeletal muscle cell, endocrine cell, glial cell, neuron, secretory cell, barrier function cell, contractile cell, absorptive cell, mucosal cell, limbus cell (from cornea), stem cell (totipotent, pluripotent or multipotent), unfertilized or fertilized oocyte, or sperm.

Moreover, methods can be implemented with or in plants or parts of plants, including fruit, flowers, leaves, stems, seeds, cuttings. Plants can be agricultural, medicinal, or decorative.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Atox1 & CCS

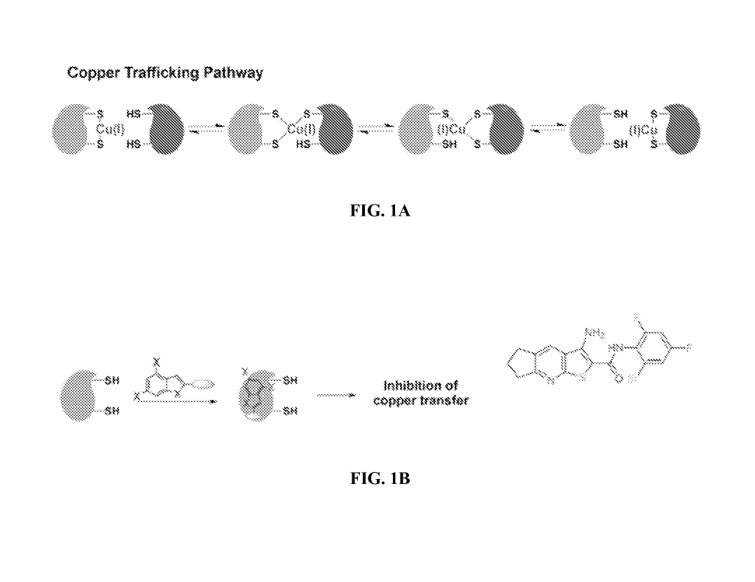

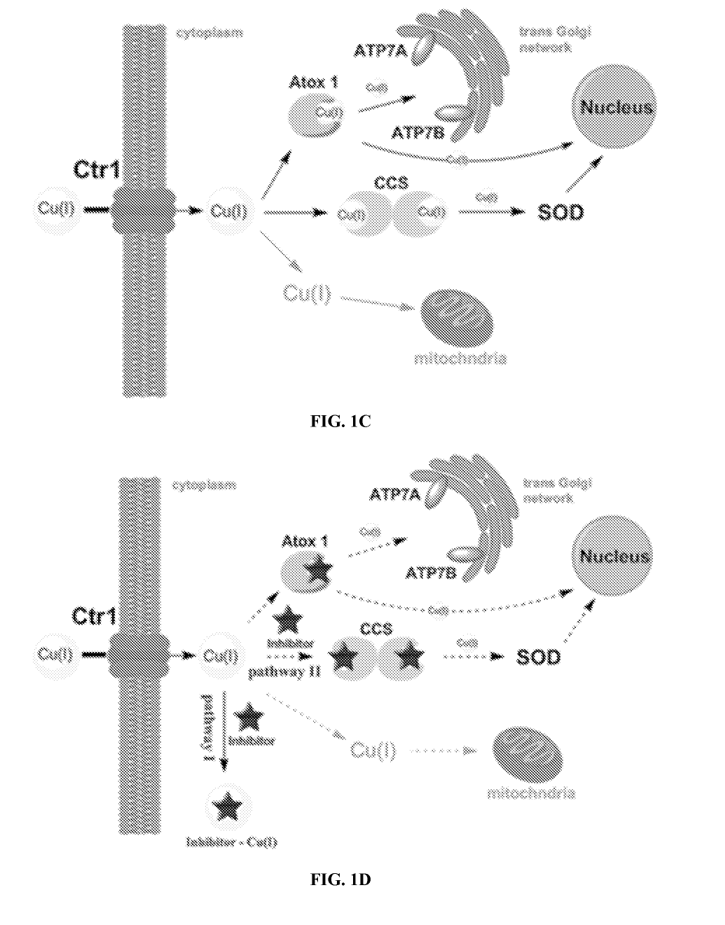

The pathways by which copper enters the human cell and is transported to specific locations are crucial to understanding copper homeostasis. The transporter protein CTRL plays a key role in high-affinity copper uptake. Once copper enters into cytoplasm, it is bound by the cytosolic copper chaperones, CCS and Atox1, which then transfer copper to specific cellular destinations. FIG. 1A illustrates the transfer of copper from a copper chaperone (left) to a proteins by a di-cysteine transfer mechanism. By inhibiting copper chaperones with small molecules like compound 50, copper transfer is inhibited (FIGS. 1B and 1D). Atox1 binds copper(I) with a conserved COX motif and delivers it to the single N-terminal metal binding domains of ATP7B and ATP7A in the secretory pathway (Lutsenko et al. 2008), which includes the trans-Golgi network (Hu 1998, Hung et al. 1998, Lin et al. 1997, FIG. 1C). Additionally, CCS (Culotta et al. 2006) has two domains with the first domain, which is structurally homologous to the Atox1, delivering copper to the antioxidant enzyme Cu/Zn superoxide dismutase (Bertini et al. 1998). Cu/Zn superoxide dismutase (SOD-1) is a key enzyme in the dismutation of the potentially toxic superoxide radicals into hydrogen peroxide and dioxygen. Since angiogenesis is characterized by proliferating endothelial cells and reoxygenation, recent studies suggest that the inhibition of SOD-1 diminishes the ability of endothelial cells to confront the increased level of ROS during angiogenesis, resulting in inhibition of angiogenesis, tumor development and metastasis (Marikovsky 2002, Fotsis et al. 1994, Huang 2000). Also, targeting these non-oncogene dependencies in the context of a transformed genotype may result in a synthetic lethal interaction and the selective death of cancer cells. Some cancer cells have higher levels of CCS and Atox1, suggesting higher dependence of cancer cells on copper trafficking A simple analysis comparing tumor and corresponding normal tissues revealed to us that the mRNA levels of human copper trafficking proteins Atox1 and CCS tend to be upregulated in most human tumors (Shin 2011, FIGS. 2A-B).

### Docking Strategy

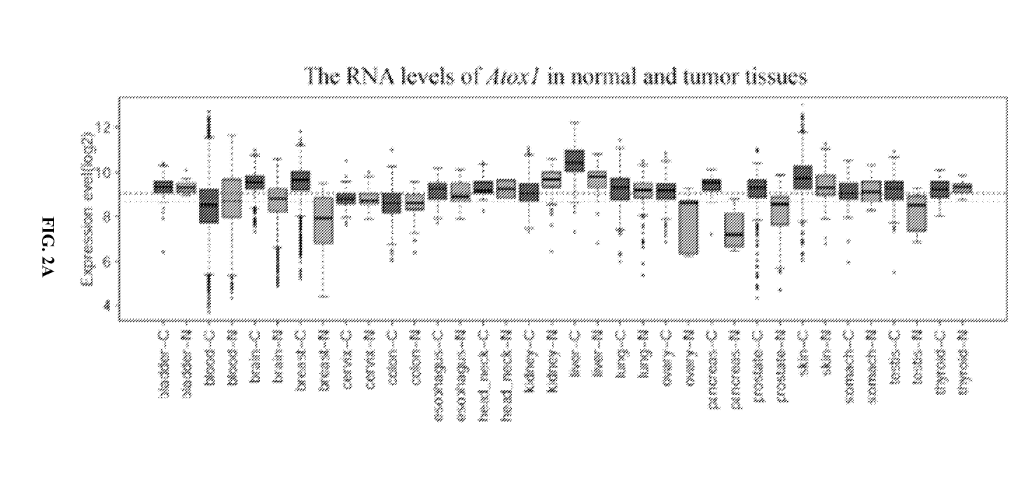

The crystal structure of Cu-Atox1 reveals a copper ion coordinated by cysteine residues from two Atox1 molecules (Wernimont et al. 2000, Anastassopoulou et al. 2004). The contact interface of these two Atox1 is a groove, which is also the protein-protein interaction interface utilized in copper delivery through Atox1 (Boal and Rosenzweig 2004). Based on this previous structural characterization, small molecules were designed that can functionally suppress copper trafficking. The copper-trafficking inhibition is achieved by targeting the protein-protein interaction interface, which is essential to the activity of copper-dependent enzymes. A hierarchical docking strategy was adopted: DOCK4.0 was used for screening of the Specs database, which contains more than 200,000 compounds. Based on the results of this screen and the consideration of structural features, physical chemistry properties, and drug-like characteristics, 237 compounds were selected for further bioactivity testing (FIG. 3).

### Lead Validation

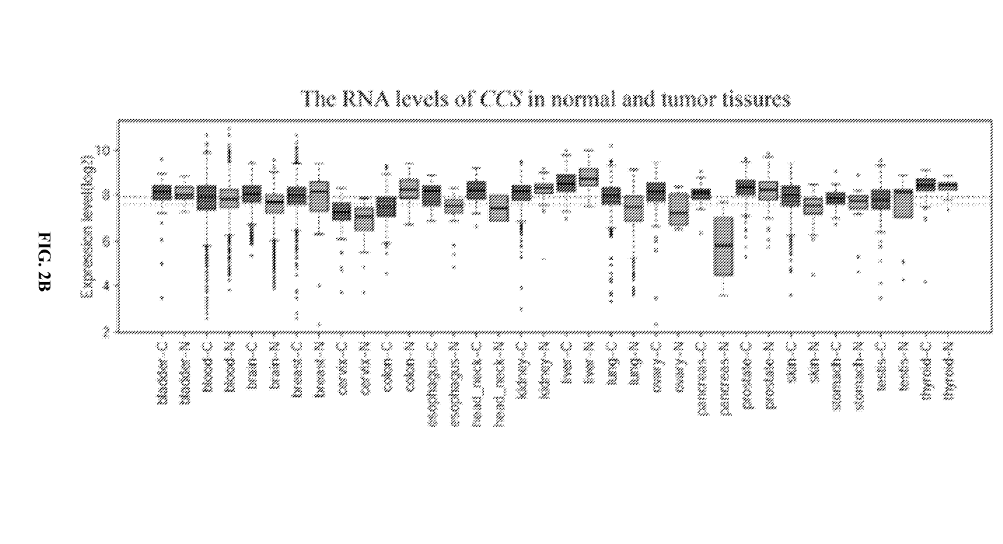

To validate the small molecules identified in the virtual screening, a previously developed FRET-based probe (Vinkenborg et al. 2009) was employed to examine inhibition effects of the 237 compounds on Atox1-based copper trafficking. The eCALWY3 probe used in this study consists of Atox1 and its copper-binding partner of the domain 4 of ATP7B (WD4) fused to green fluorescent proteins as partners of FRET and connected through a long flexible linker; Atox1 delivers copper(I) to WD4 through protein-protein recognition and copper(I) exchange via conserved Cys residues in both proteins (Banci et al. 2008). Binding of either copper(I) or zinc(II) is known to induce a 2-fold decrease of the FRET ratio of eCALWY3 although copper(I) is the biologically relevant metal of this complex (Vinkenborg et al. 2007). A small molecule that specifically binds at the interface inhibits metal binding by the complex which increases FRET ratio to the same level of that of the apo-form eCALWY3 (FIG. 4). Six compounds (at 100 μM) exhibited inhibition of the Atox1-WD4 interaction in the presence of metal (FIG. 5) and induced almost full recovery of the FRET ratio back to that of the apo form (Table 1), suggesting that their K_{d}s to Atox1-WD4 should be below 100 μM.

TABLE 1

| | | Fluorescence | |
|---|---|---|---|
| | | Intensity Ratio | Recover |
| Molecule | Molecular Structure | (Citrine/Cerulean) | % |
| LC-21 Compound 2 | 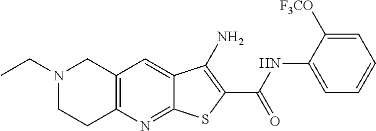 [SMILES: CCN1CCc2nc3sc(C(=O)Nc4ccccc4OC(F)(F)F)c(N)c3cc2C1] | eCALWY3 only: 3.54 100 μM compound added: 3.05 | 86% |
| Compound 30 | 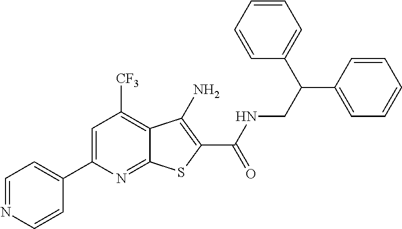 [SMILES: Nc1c(C(=O)NCC(c2ccccc2)c2ccccc2)sc2nc(-c3ccncc3)cc(C(F)(F)F)c12] | eCALWY3 only: 2.27 100 μM compound added: 1.53 | 67% |
| LC-42 Compound 49 | 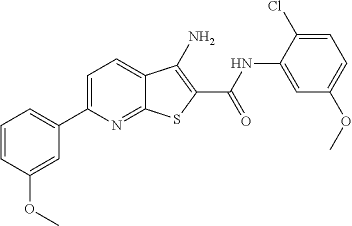 [SMILES: COc1cccc(-c2ccc3c(N)c(C(=O)Nc4cc(OC)ccc4Cl)sc3n2)c1] | eCALWY3 only: 2.24 100 μM compound added: 1.89 | 84% |
| LC-1 Compound 50 | 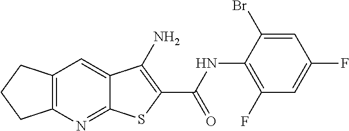 [SMILES: Nc1c(C(=O)Nc2c(F)cc(F)cc2Br)sc2nc3c(cc12)CCC3] | eCALWY3 only: 3.58 100 μM compound added: 43 | 96% |
| LC-40 Compound 61 | 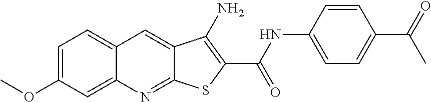 [SMILES: COc1ccc2cc3c(N)c(C(=O)Nc4ccc(C(C)=O)cc4)sc3nc2c1] | eCALWY3 only: 3.68 100 μM compound added: 2.72 | 74% |
| LC-44 Compound 71 | 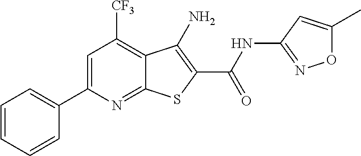 [SMILES: Cc1cc(NC(=O)c2sc3nc(-c4ccccc4)cc(C(F)(F)F)c3c2N)no1] | eCALWY3 only: 2.25 100 μM compound added: 1.86 | 83% |

### Cell Viability and Relative Kd Values

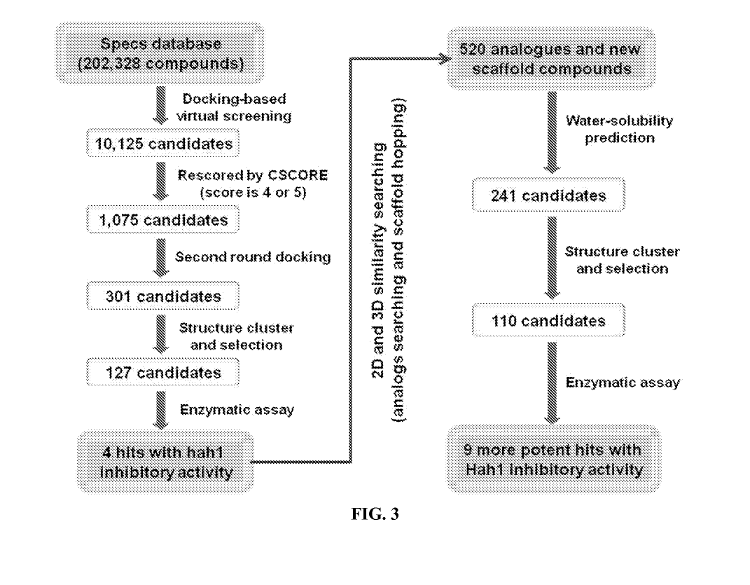

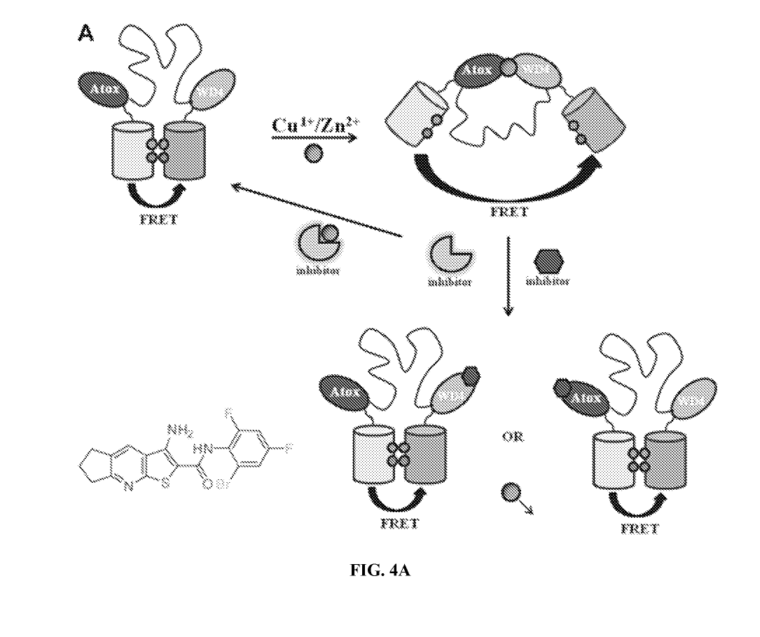

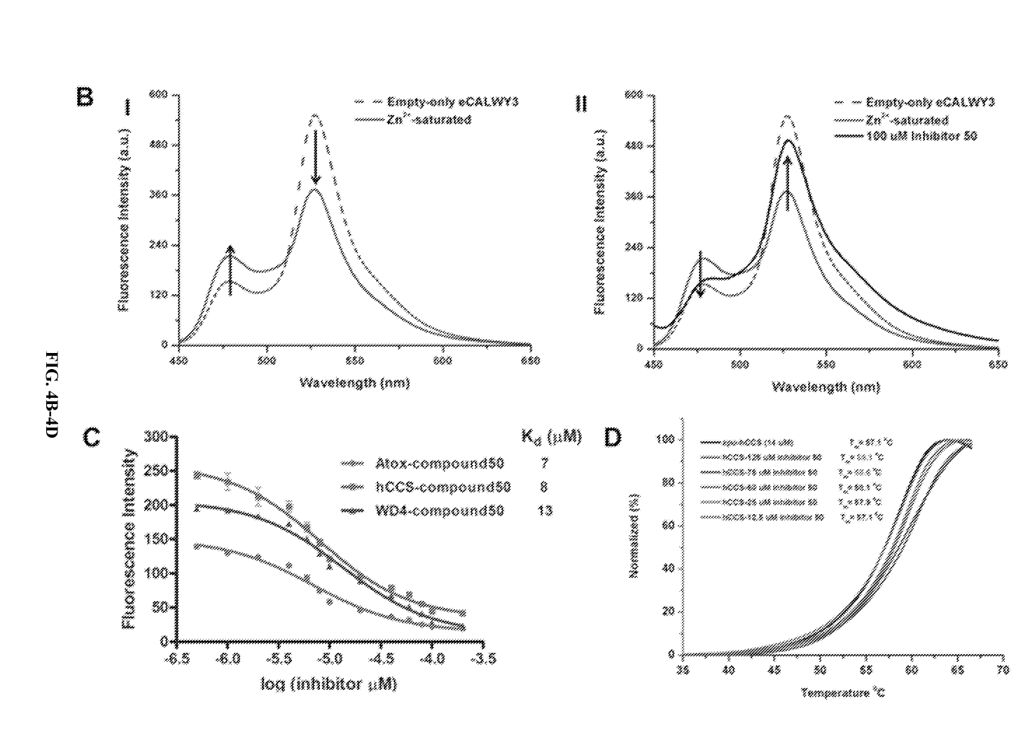

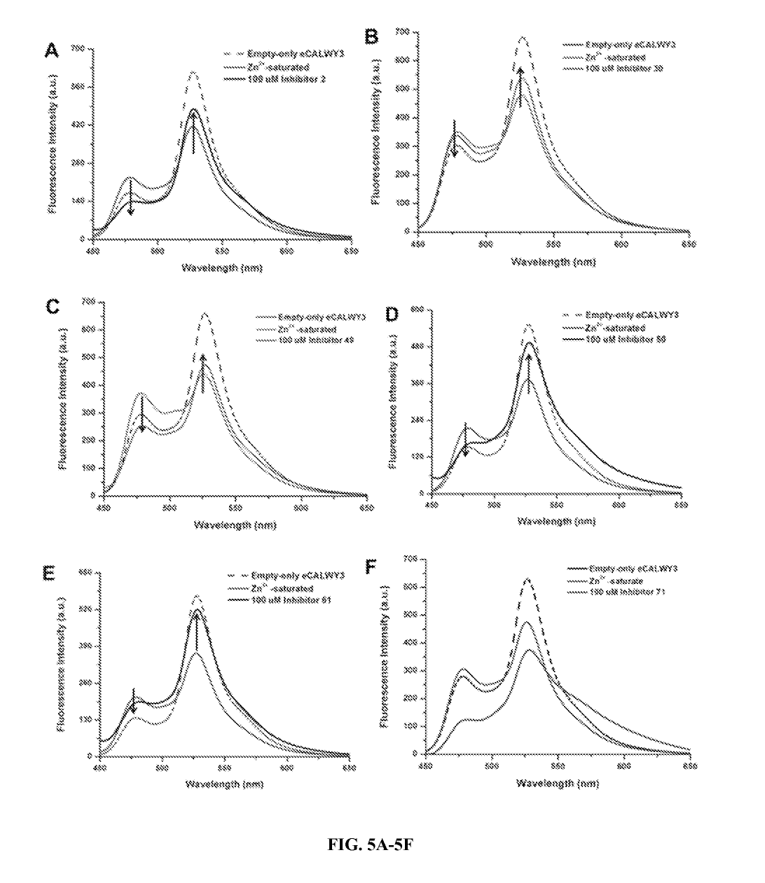

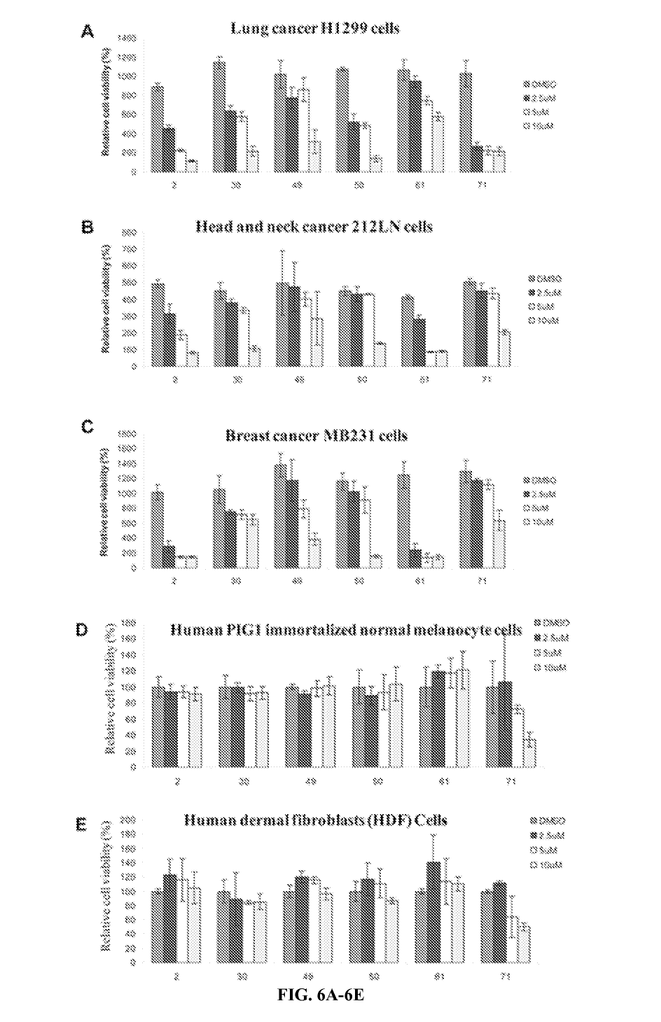

Six compounds were evaluated for their effects on the viability of cultured cancer cells and normal cells (FIG. 6). Treatment with these compounds markedly induced cell death in several cancer cell lines tested (Lung cancer H1299 cell, Head and neck cancer 212LN cell, Breast cancer MB231 cell) (FIGS. 6A-C). When primary normal cells (human PIG1 cell and human dermal fibroblast cell) with diverse proliferative capacities were incubated with highly purified compounds for 72 hours, there was little apparent reduction in cell viability at the highest concentration tested (10 μM, FIGS. 6D-E) except compound 71 which appears to be toxic to normal cells. FIGS. 6A-E demonstrate that compounds 2, 30, 49, 50, and 61 induce cell death in cancer cells and are not harmful to normal cells.

The relative binding affinities of 2, 50 and 61 were obtained from the binding studies based on the FRET assays (FIGS. 7A-D).

### Binding Affinity for Human CCS

Besides Atox1 human CCS (hCCS) is another main copper chaperon protein (Culotta et al. 2006, Kawamata and Manfredi 2008, Wood and Thiele 2009). An alignment of Atox1, WD4, and the copper-binding domain 1 of hCCS shows that residues in copper-binding and protein-protein interaction are well conserved (FIGS. 8A and 8B). Based on this similarity, the intrinsic fluorescence of tyrosine for Atox1 and WD4 and tryptophan for hCCS upon addition of small molecules was monitored. The K_{d}s of the small molecules to these three proteins were measured to be ˜7-18 μM (FIG. 4C and FIGS. 8D and 8F). Compound 50 binds Atox1 with a K_{d} of 7 μM and hCCS with a K_{d} of 8 μM. The binding affinities were further confirmed by using isothermal titration calorimetry (ITC). A fluorescence-based thermal shift assay was also employed to further test the interaction of the compounds and hCCS. As shown in FIG. 4D, the presence of compound 50 shifted the melting temperature (Tₘ) of the native hCCS protein by approximately 2° C. when only 9-fold excess of compound was added, indicating a significant interaction between the two. Compound 2 and 61 also shifted the melting temperature (Tₘ) of hCCS by approximately 1-1.5° C. when 9-fold excess of compound was added (FIGS. 8E and 8G). The biophysical analyses indicate that these small molecules bind Atox1 and hCCS and the binding disrupts simultaneous metal binding by both proteins based on the FRET-based assay.

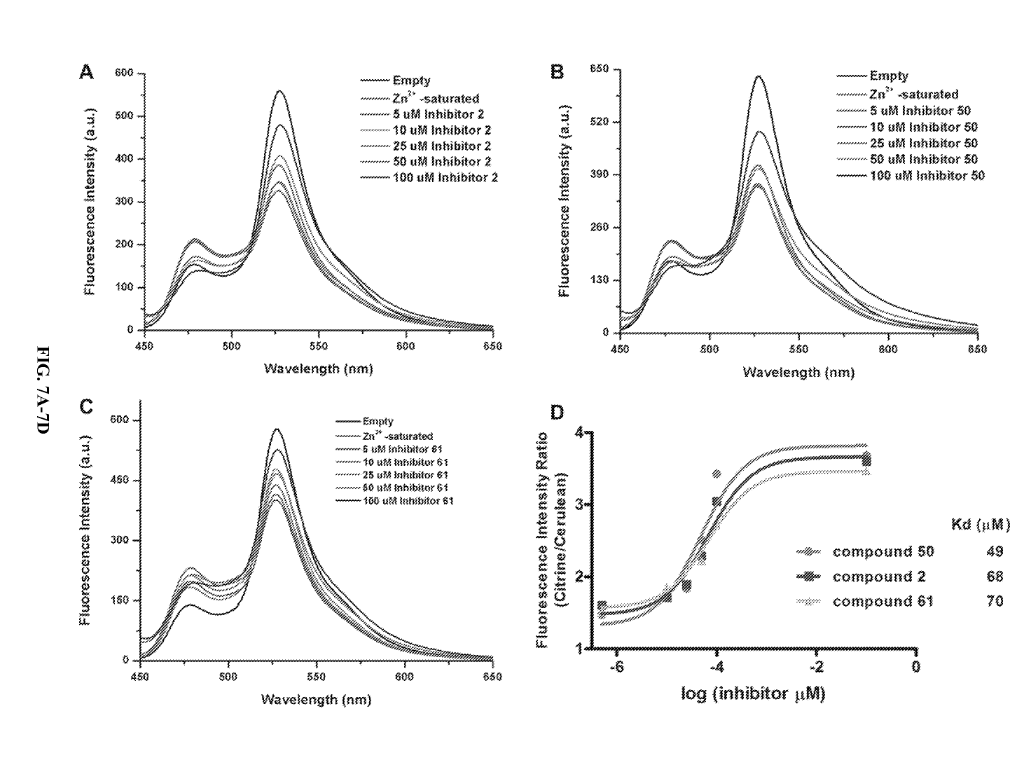

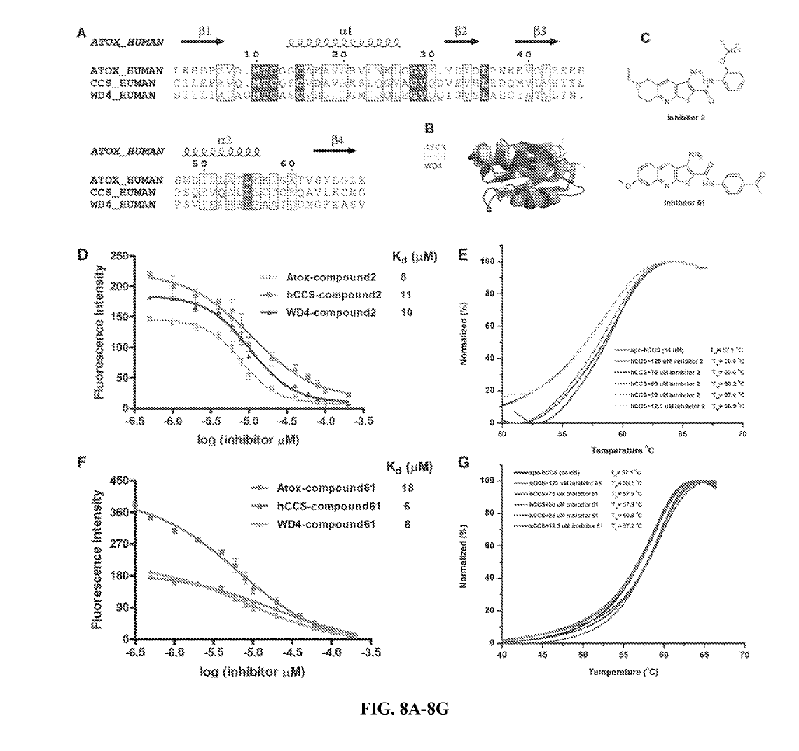

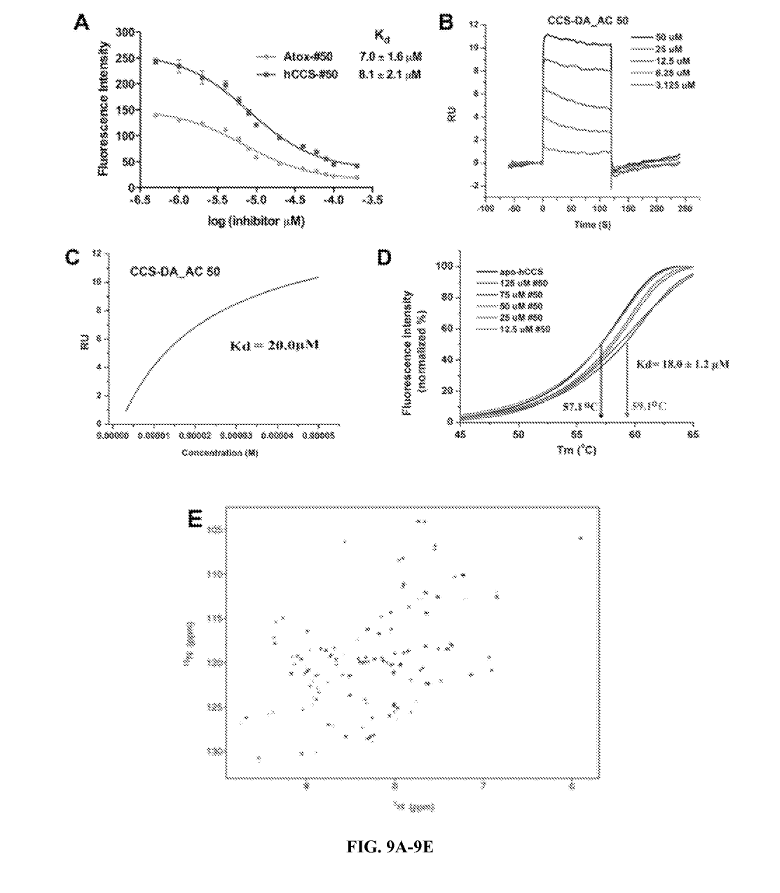

Given the similarity between copper-binding and protein-protein interaction residues of Atox1 and the copper-binding domain 1 of CCS, Atox1 and CCS were expressed and purified in order to further test their binding to compound 50. The binding constants (K_{d}s) were determined to be ˜7.0 μM for Atox1 and ˜8.1 μM for CCS, respectively (FIG. 9A). The binding assay was performed in an SPR assay. Varied concentrations of compound 50 (from 3.2 μM to 50 μM) were used with 1 μM hCCS (FIGS. 9B-C). In order to confirm the stabilization of CCS by compound 50, a thermal shift assay was performed, resulting in a similar Kd value. Graphs indicate unfolding transition of 14 μM CCS in the presence of 12.5, 25, 50, 75, and 125 μM of compound 50, respectively (FIG. 9D).

NMR was used to characterize the interaction of Cu(I)-loaded Atox1 with compound 50. Superposition of ¹H-¹⁵N HSQC spectra for Cu(I)-loaded Atox1 with (red) or without (black) compound 50. Protein samples were freshly made at a concentration of 20 mM in 50 mM HEPES, 200 mM NaCl, 1 mM DTT, pH 7.0. The stock of compound was added to the final protein-to-compound ratio of 1:5. Experiments were carried out at 25° C. on a Bruker Avance III 600 MHz NMR spectrometer equipped with a TCI cryoprobe (FIG. 9E).

### Molecular Modeling

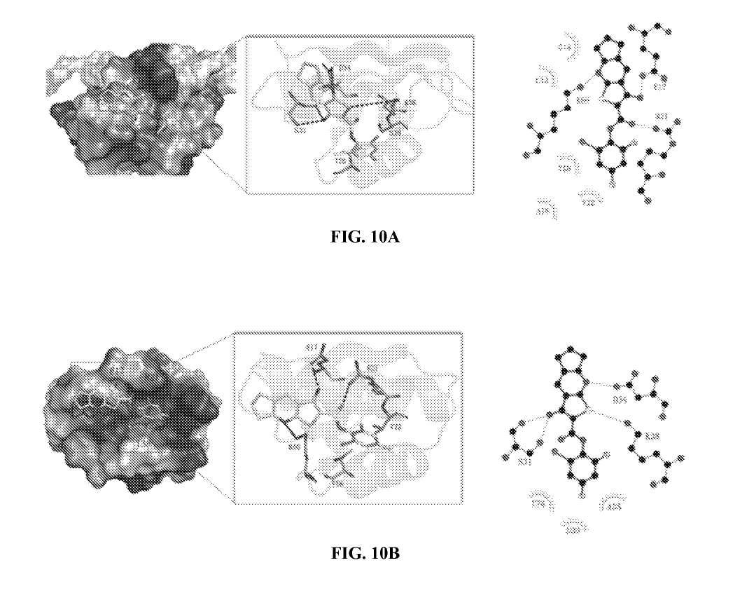

Molecular modeling was performed in order to understand the binding mode of the small molecule to both Atox1 and hCCS. Computational molecular docking was applied for compound 50 to model binding to Atox1 and hCCS. For Atox1, hydrogen bonds between heteroatoms of compound 50 and side chains of Glu17, Arg21, Lys60 were revealed. Upon docking into hCCS, compound 50 displayed interactions with amino acids Ser31, Asp34, and Lys38. Additionally, the structural model suggested hydrophobic packing of the substituted phenyl group of compound 50 with Va122 and Thr58 of Atox1 and Ser39 and Thr76 of hCCS (FIGS. 10A-B)

### Single and Double Mutant Studies

To validate the computational model, several Atox1 mutants were constructed replacing residues E17A, R21A, K60A, T58A and V22S with Ala (predicted locations of mutated residues illustrated in FIG. 11A). As shown in FIG. 11B, the single mutations of these amino acids weakened the binding affinity of compound 50 to the mutant protein as compared to the wild-type Atox1(4-6 fold). FIG. 11D illustrates binding affinities for hCCS with single mutations. To further validate these binding sites, the binding of compound 50 to several double mutations (E17R21A, E17T58A, E17K60A, R21K60A and R21AV22S) was tested. The binding affinity of these mutants was further weakened (5-8 fold) as compared to the wild-type Atox1 and hCCS, supporting involvement of these residues in small molecule binding by Atox1 (FIGS. 11C and 11E).

### Effect on Copper Uptake

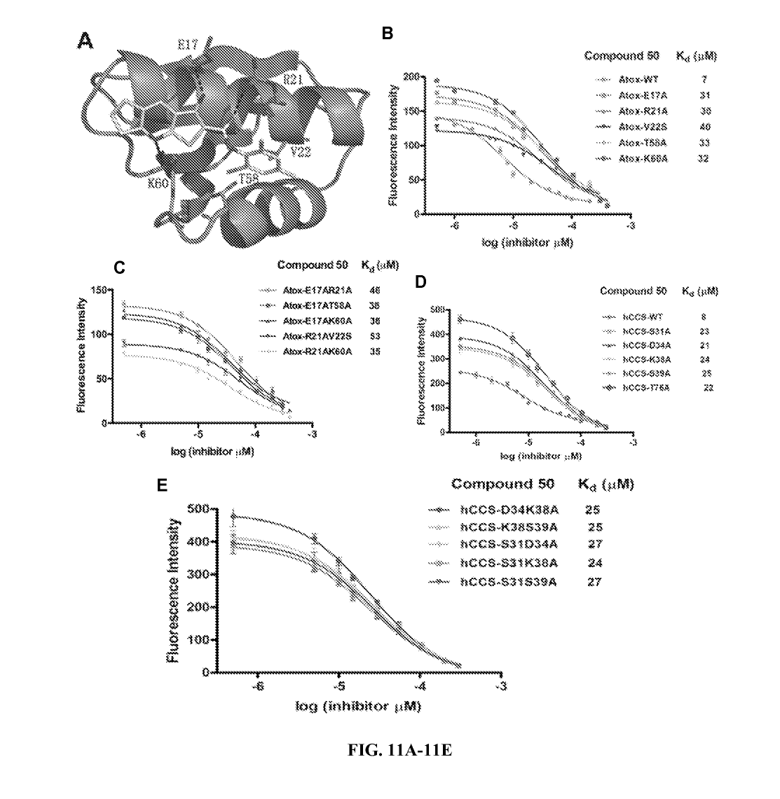

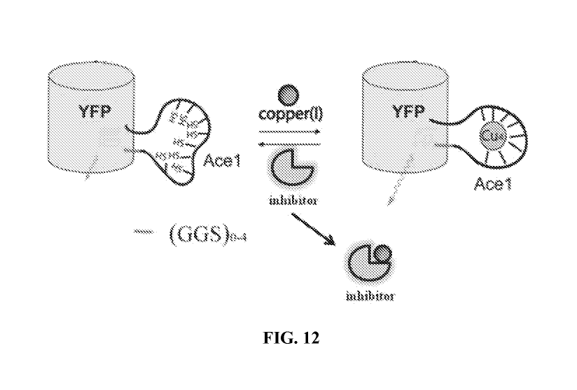

A genetically encoded copper (I) probe capable of monitoring copper fluctuations inside living cells was used to study effect of compound 50 on copper uptake by mammalian cells. HeLa cells were used in the initial test. CuSO₄ (150 μM) was added to the medium and cells were allowed for incubation for 10 min. A clear fluorescence reduction was observed, indicating an increased intracellular copper level. Compound 50 (50 μM) was added at this time point (10 min after copper addition) to the same medium, a fluorescence increase was observed, suggesting reduced copper uptake in the same cells. Real-time imaging of Hela cells with 150 μM CuSO₄ and 50 μM compound 50 was performed following the same procedure, which demonstrated a rapid inhibition effect of compound 50 on copper uptake in living cells (FIG. 12).

Compound 50 Inhibits Cancer Cell Proliferation with Minimal Effects on Noncancerous Cells

Compound 50 demonstrated a high efficiency in inhibiting cancer cell proliferation in a dose-dependent manner (FIG. 13A) with minimum effects on noncancerous cell lines observed (FIG. 13B). Using Western blotting, both Atox1 and CCS are expressed at higher levels in selected cancer cells compared to normal cells (FIG. 13C).

Atox1 and/or hCCS Knockdown

Knockdown of either Atox1 or CCS in H1299 lung cancer cells was performed using short hairpin RNA (shRNA). A decreased cell proliferation was observed in both cases (FIG. 13D), indicating that both proteins may indeed play important roles in cancer proliferation. To further confirm Atox1 and hCCS as cellular targets of compound 50, inhibition of cell viability in H1299 cells with stable knockdown of either Atox1 or hCCS, as well as both proteins was studied. Cells with stable knockdown of either Atox1 or hCCS were still susceptible to the treatment of compound 50; however, double knockdown of Atox1 and hCCS resulted with the inability of compound 50 to inhibit cell proliferation (FIG. 13E). Note that the relative cell viability compared to untreated state was shown in these studies. These results compellingly implicate Atox1 and hCCS as the means through which compound 50 exerts its inhibitory effect on cell proliferation.

### In Vivo Studies

An in vivo small molecule treatment experiment was performed to further test the efficacy of these compounds. Initial toxicity studies by chronic injection of compound 50 to nude mice for 4 weeks revealed that 100 mg/kg/day (administered intraperitoneally) is a well-tolerated dose. In addition, continuous treatment with compound 50 (100 mg/kg/day) for 7 days did not result in significant alteration to body weight, complete blood cell counts, or hematopoietic properties of nude mice (Table 2).

TABLE 2

| | Reference range | Control day | 100 mg/kg/day |
|---|---|---|---|
| Test name (units) | (low-high) | 30 (mean) | (mean) |
| WBC (×10³/μL) | 2.6-10.1 | 7.66 ± 1.64 | 6.89 ± 3.125 |
| LYM (×10³/μL) | 1.3-8.4 | 4.685 ± 0.88 | 4.74 ± 1.556 |
| MONO (×10³/μL) | 0-0.3 | 0.475 ± 0.35 | 0.44 ± 0.17 |
| GRAN (×10³/μL) | 0.4-2 | 2.42 ± 0.38 | 1.58 ± 1.4 |
| HCT (%) | 32.8-48 | 49.6 ± 3.82 | 30.85 ± 4.879 |
| MCV (fl) | 42.3-55.9 | 58.05 ± 1.06 | 56.75 ± 0.919 |
| RDWa (%) | 0-99.9 | 16.3 ± 0.57 | 21.5 ± 4.667 |
| HGB (g/dl) | 10-16.1 | 12.85 ± 0.78 | 7.45 ± 1.768 |
| MCHC (g/dl) | 29.5-35.1 | 25.9 ± 0.42 | 24 ± 1.98 |
| RBC (×10⁶/μL) | 6.5-10.1 | 8.545 ± 0.81 | 5.43 ± 0.778 |
| MCH (pg) | 13.7-18.1 | 15.05 ± 0.49 | 13.6 ± 1.273 |
| PLT (×10³/μL) | 250-1540 | 1189.5 ± 38.9 | 742 ± 684.5 |
| MPV (fl) | 0-99.9 | 6.35 ± 0.49 | 6.35 ± 0.495 |

### Xenograft Results

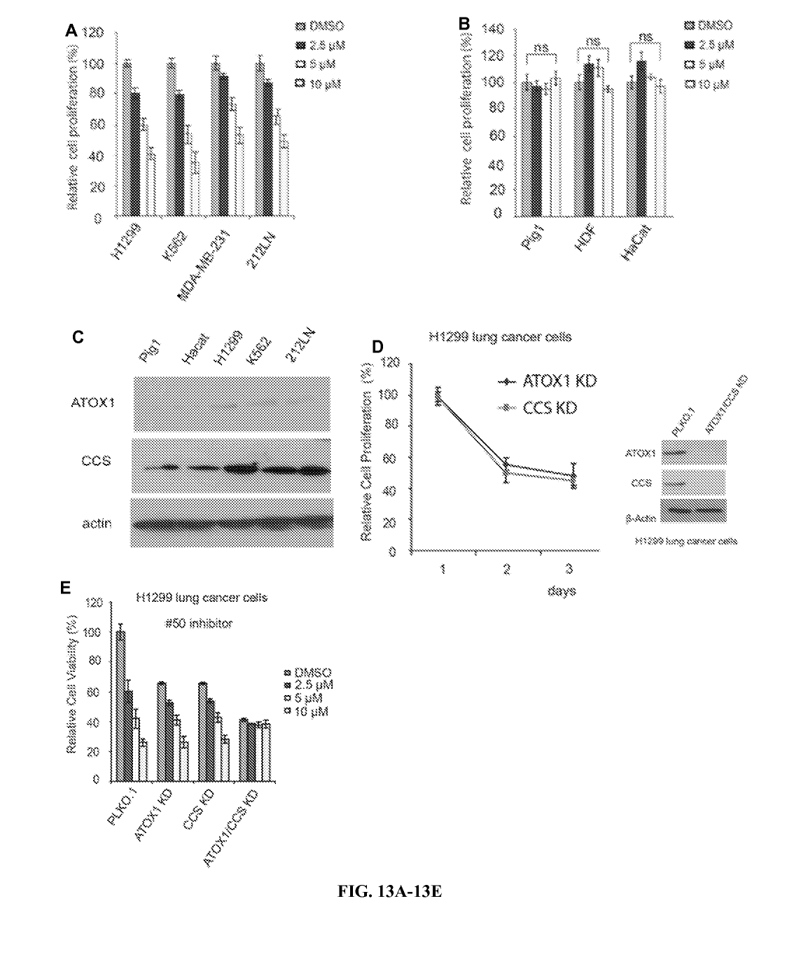

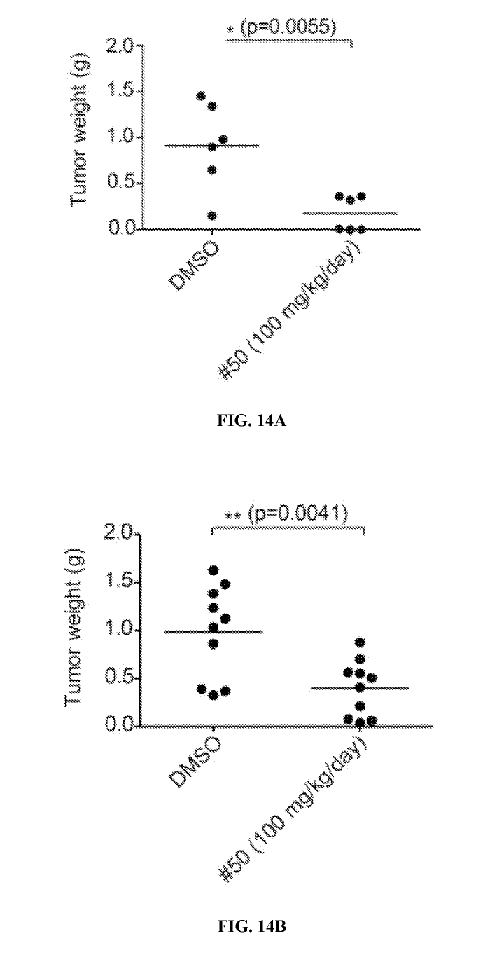

Xenograft experiments were performed by injecting H1299 or K562 cells into nude mice as previously described (Hitosugi et al. 2009). Six days post-injection, mice were divided into two groups (n=10/group) and treated with either compound 50 (100 mg/kg/day) or vehicle control for 21 days. Treatment with compound 50 results in significantly decreased tumor growth and size in comparison to mice receiving vehicle control (FIGS. 14A-B). The data suggests compound 50 targets Atox1 and hCCS in vivo, and this inhibition causes specific toxicity to tumor cells. The results show that the protein levels of Atox1 and hCCS are higher in cancer cells compared to normal cells, suggesting higher dependence on copper of cancer cells (FIG. 12C). Targeting Atox1 and CCS by compound 50 can effectively suppress tumor growth without affecting normal tissues in mice. Besides being an important metal in various essential (cytochrome c oxidase) and important enzymes (Cu/Zn superoxide dismutase), the significant roles of copper in angiogenesis as endothelial cell growth and cell proliferation all suggest potential higher dependence of cancer cells on copper for survival and proliferation. Therefore, small molecules that inhibit cellular copper uptake can be a powerful approach in cancer therapy. These molecules could also be used to treat Wilson's diseases or used in wound healing processes.

Investigation of Additional Pathways that Contribute to Compound 50's Inhibition of Cancer Cell Proliferation

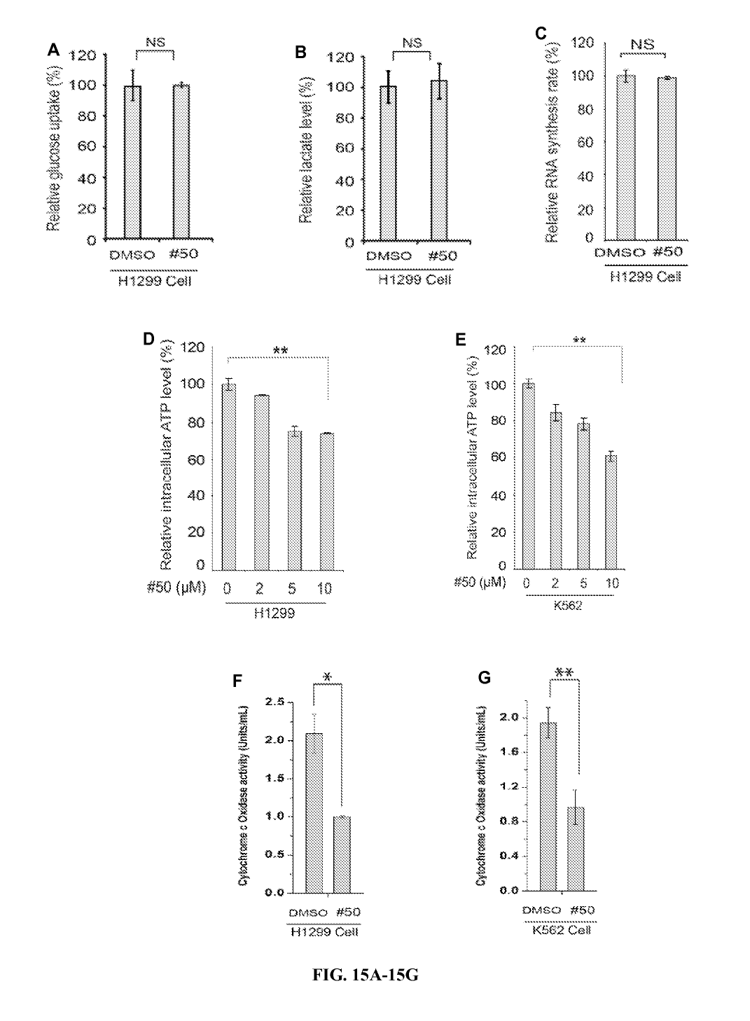

The effect of compound 50 on cellular levels of lactate, glucose uptake, and glucose-dependent RNA synthesis was examined, but no noticeable changes were observed (FIGS. 15A-C). However, compound 50 significantly reduced cellular ATP levels (FIGS. 15D-E). Inhibition of copper trafficking reduces the cellular ATP level; the reduced ATP level results in activation of AMP-activated protein kinase (AMPK) that leads to reduced lipogenesis and contributes to the inhibition of cancer cell proliferation.

Copper is an essential co-factor for electron transfer and oxygen reduction activity of cytochrome c oxidase. Disruption of oxidative phosphorylation (OXPHOS) has been tied to to increased ROS level and reduced ATP production (Wallace 2012). Both of these effects were observed upon treatment with compound 50. Although the exact mechanism for copper delivery to cytochrome c oxidase (CCO) remains unclear, previous reports have shown that an ATP7A (the copper delivery target of Atox1) defect led to reduced CCO activity (Mercer, 1998). The involvement of CCS in copper delivery to mitochondria has also been implicated (Kim 2008). Therefore, reduced OXPHOS activity is expected when Atox1 and CCS are inhibited. The CCO activities (Units/mL) of both H1299 and K562 cells were significantly lowered in the presence of compound 50 compared to the control (FIGS. 15F-G).

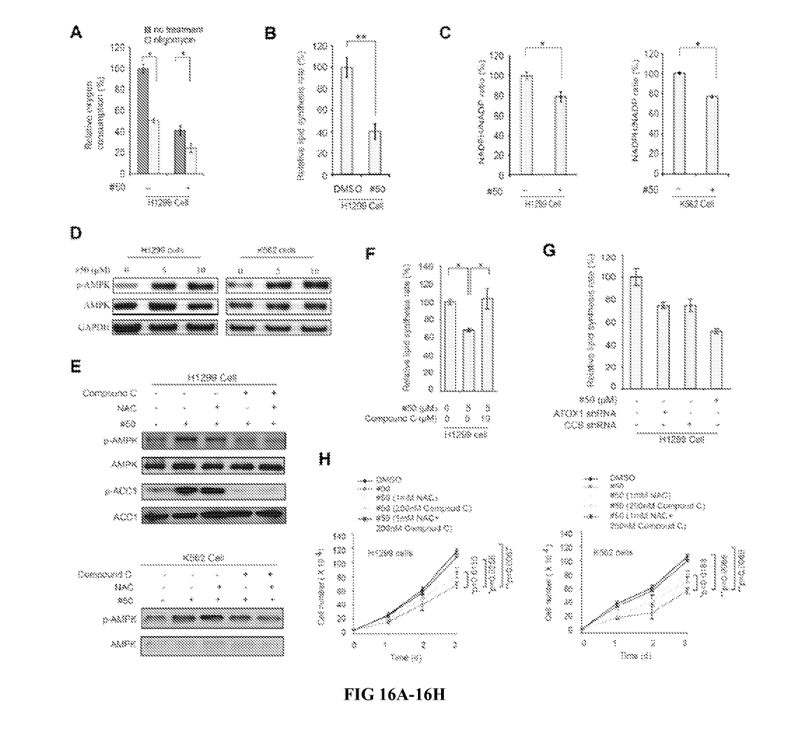

Mitochondrial performance of H1299 cells upon treatment with compound 50 was examined. Compound 50 significantly decreased oxygen consumption rate in the presence or absence of ATP synthase inhibitor oligomycin (FIG. 16A). Compound 50 caused significant decreases in lipid synthesis and the NADPH/NADP⁺ ratio in the H1299 cancer cells (FIGS. 16B-C), a finding that strongly supports reduced lipogenesis as a key factor in cell proliferation inhibition. Reduced levels of ATP result in activation of the AMP-activated protein kinase (AMPK) (Mihaylova 2011, Hardie 2012), a central sensor of cellular metabolism, which should subsequently lead to increased phosphorylation of its direct target, acetyl-CoA carboxylase 1 (ACC1), and inhibition of lipid biosynthesis (Jiang et al. 2013, Scott 2012). Treatment with compound 50 increased the levels of AMPK phosphorylation and ACC1 phosphorylation (FIGS. 16D-E). These effects could not be rescued with the ROS scavenger NAC; however, treatment of an AMPK inhibitor compound C (CAS No. 866405-64-3) together with compound 50 almost completely reversed the increased phosphorylation on both proteins and recovered lipid synthesis in H1299 cells (FIGS. 16E-F). Similar effects were also observed in K562 cells.

Subsequent investigations revealed that decreased lipid synthesis was observed in either Atox1 or CCS knockdown cells (FIG. 16G). Importantly, treatment with either N-acetyl-L-cysteine (NAC) or compound C partially rescued cell proliferation caused by compound 50 (FIG. 16H). When cells were treated with both NAC and compound C, we observed almost complete rescue of cell proliferation inhibition induced by compound 50 (FIG. 16H). These results further support that the inhibition of copper trafficking induces an increased ROS level and AMPK activation (through reduced ATP production), which attenuate cancer cell proliferation and tumor growth.

### Induction of Cellular Oxidative Stress

Inhibition of copper trafficking induces cellular oxidative stress which contributes to the inhibition of cancer cell proliferation. Copper is essential for the activity of human superoxide dismutase (SOD). FIG. 17 is a mechanistic model of cancer cell proliferation inhibition through targeting of copper trafficking proteins Atox1 and CCS (upregulated in cancer cells). Selective inhibition of copper trafficking proteins Atox1 and CCS by compound 50 elevates cellular ROS level and reduces lipogenesis through AMPK activation.

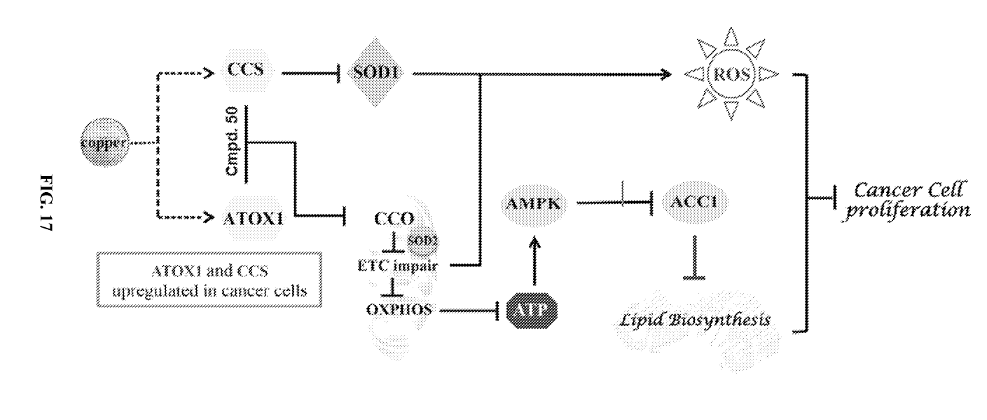

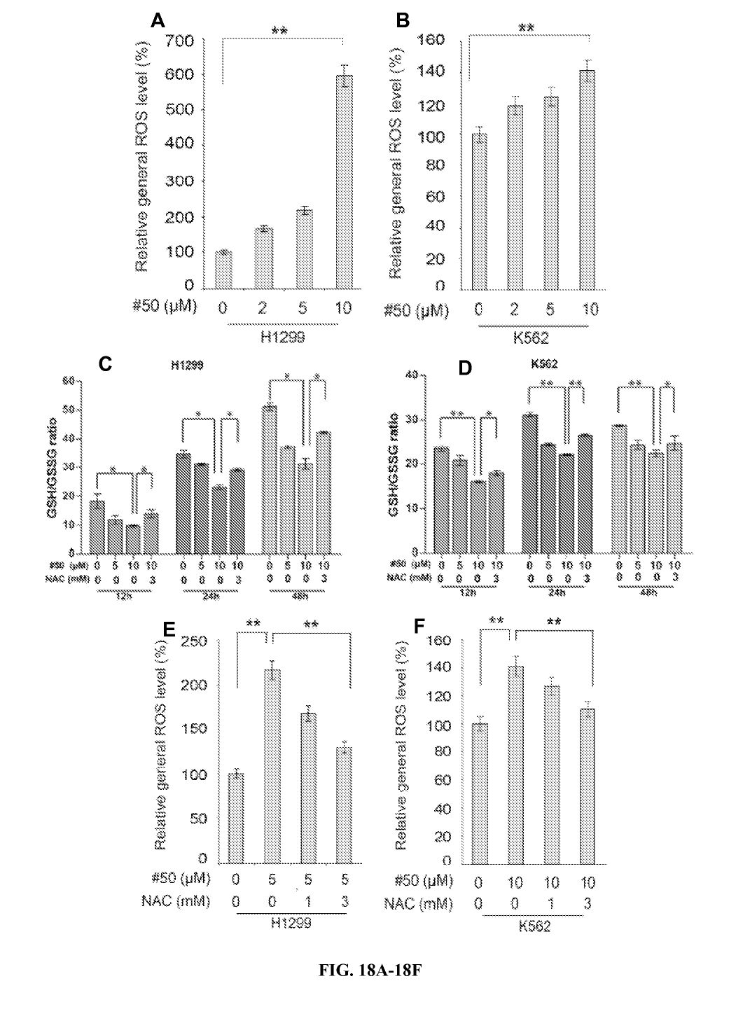

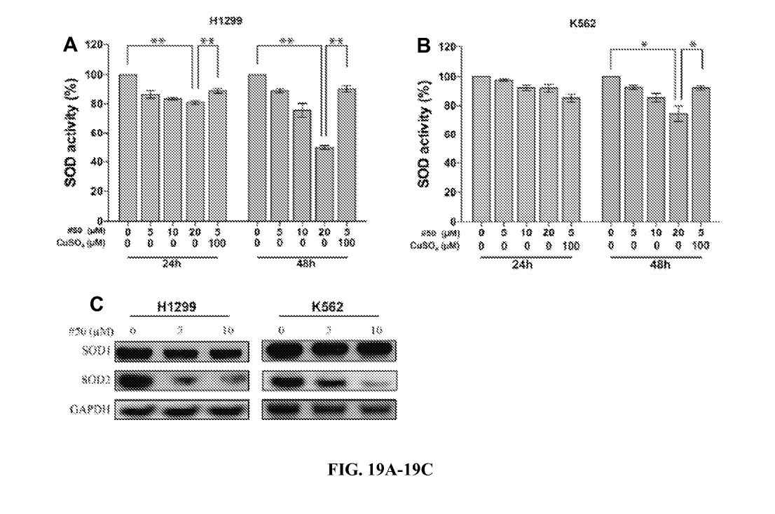

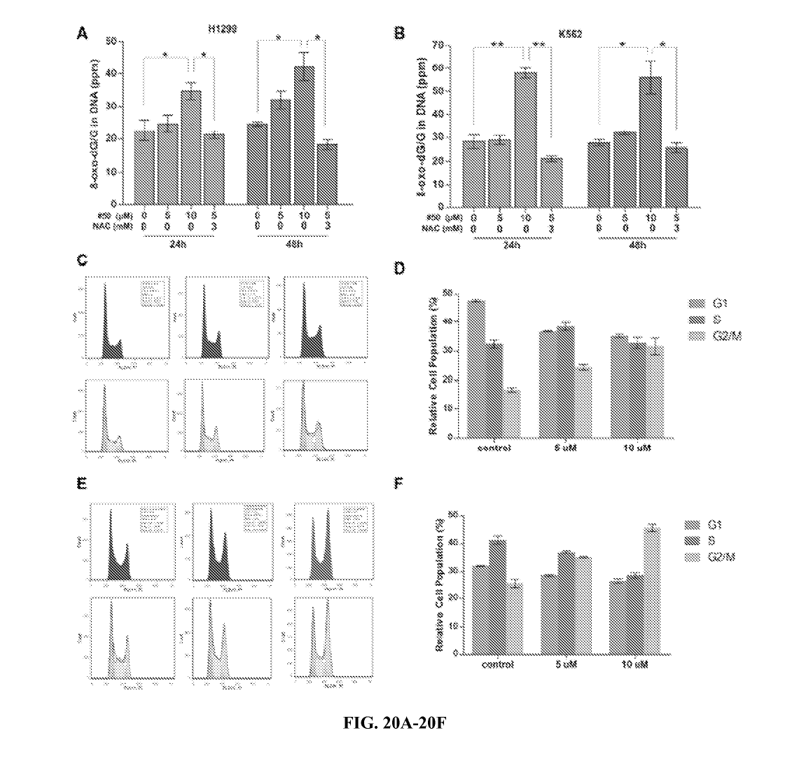

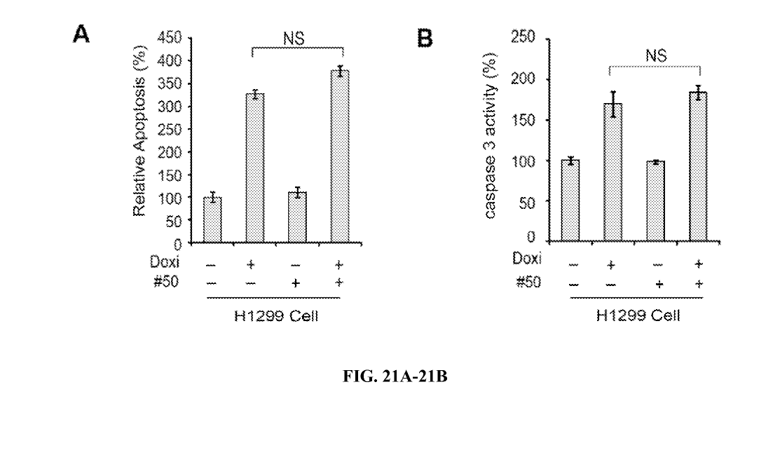

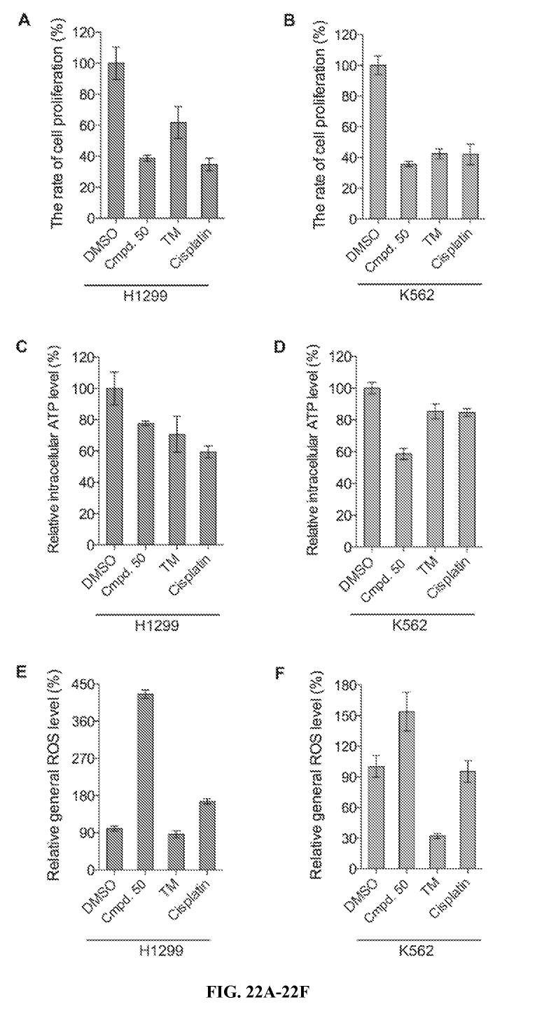

Treatment of cells (H1299 and K562) with compound 50 (10 μM) led to increased cellular ROS level (FIGS. 18A-B), accompanied by a decrease of the ratio of reduced to oxidized glutathione (GSH/GSSG) in both cells (FIGS. 18C-D). These effects can be almost completely rescued with the treatment of ROS scavenger N-acetyl-L-cysteine (NAC) (FIGS. 18E-F). In agreement with previous studies (Rae 1999), the inhibition of CCS didn't decrease the SOD1 expression level but led to reduced SOD1 activity and decreased SOD2 level (FIGS. 19A-C). The increased oxidative stress was further confirmed with the observation of noticeably increased levels of 8-OHdG in genomic DNA, one of the major products of DNA oxidation, after compound 50 treatment (FIGS. 20A-B). The oxidative stress also caused a G2/M phase cell cycle arrest in both H1299 and K562 cancer cells (FIGS. 20C-F). Interestingly, the treatment of compound 50 didn't noticeably induce apoptosis (FIGS. 21A-B).

In summary, Atox1 and CCS are upregulated in most cancer cells. A small molecule that specifically inhibits copper chaperones Atox1 and CCS results in significantly reduced cancer cell proliferation and tumor growth. Mechanistic investigations reveal that the inhibition of copper trafficking leads to increased ROS and reduced lipid synthesis, which explain the reduced cancer cell proliferation (FIGS. 16H and 17). These discoveries, together with the observed upregulation of Atox1 and CCS in cancer cells, indicate a critical role of copper uptake and trafficking in cancer cell proliferation. This work establishes copper trafficking as a new pathway for future anticancer therapeutic developments.

### Virtual Screening for Compound Binding at the Atox1 Active Site

A hierarchical docking strategy was adopted: DOCK4.0 was used for initial screening on Specs database that containing more than 200,000 compounds and standard DOCK score was used to rank the result list; the top ranked 10,125 candidates were rescored by CSCORE module of SYBYL 7.3 and 1,075 compounds whose scores are 4 or 5 were selected. Then these compounds were further docked using Autodock software. According to the energy and ki value, top 301 compounds were chose and then structurally clustered to 60 clusters by pipeline pilot 7.5 program. Finally, according to the structure features, physical chemistry properties, drug-like characters etc, 127 compounds were chose for bioactivity test. Four compounds have been proven effective for hah1 inhibition.

Based on the structures of the four bioactive compounds, 2D similarity searching was conducted to discover analogues and 3D similarity mapping was adopted for scaffold hopping to get new scaffolds, both by using ChemMapper web server. Afterward, molecular water-solubility was predicted to further screen the 520 analogues and new scaffold compounds. Among these compounds, seven molecules show hah1 inhibition activities. Structure cluster and selection was applied again to ensure the structure diversity of the candidates. This procedure provides 110 candidates for enzymatic assay and six more potent hits with hah1 inhibitory activity were discovered.
Expression and Purification of Atox, hCCS and WD4
The E. coli strain BL21 was transformed with pET28a-Atox, hCCS and WD4 domain or mutants of Atox, hCCS and WD4, cultured in LB medium containing 50 mg/mL kanamycin at 37° C. to an absorbance of 0.6 at 600 nm, and induced with 1 mM IPTG for 16 hours at 16° C. before being harvested by centrifugation. The cell pellets were suspended in lysis buffer (10 mM Tris, pH 7.5, 200 mM NaCl, 1 mM DTT) and disrupted by sonication. After centrifugation, the supernatant was applied to a Ni-NTA column and proteins were eluted with elution buffer (10 mM Tris, pH 7.5, 200 mM NaCl, 1 mM DTT and 400 mM Imidazole). The 6-His tag of proteins were removed by digestion with thrombin. The samples were exchanged and further purified by the buffer using size-exclusion chromatography (S200 Sephacryl column, GE) in 50 mM HEPES, 200 mM NaCl and 1 mM DTT. Fractions containing protein were analyzed using SDS-PAGE and fractions showing a single band corresponding to the expected molecular weight were pooled, resulting in >95% pure protein samples.
Expression and Purification of eCALWY3
The protein were expressed in E. coli strain BL21 and purified according to a published method. Expression was induced using 0.1 mM IPTG, and bacteria were subsequently grown at 16° C. for 16 hrs. Lysis of bacteria was obtained by sonication and the soluble protein fraction was purified using nickel affinity chromatography and histidine tags were subsequently removed by digestion with thrombin and a second additionally purified using size-exclusion chromatography (S200 Sephacryl column, GE) in 50 mM Tris, 100 mM NaCl and 1 mM DTT, pH 7.5. Fractions containing protein were analyzed using SDS-PAGE and fractions showing a single band corresponding to the expected molecular weight were pooled, resulting in >95% pure protein samples.

### FRET Measurement

FRET for the eCALWY3 was performed in 150 mM HEPES, 100 mM NaCl, 1 mM DTT and 10% glycerol (pH 7.1). Zn²⁺ titration was done by mixing 0.9 mM of Zn²⁺ from a slightly acidic stock solution of ZnCl₂ with buffering systems consisting of 1 mM DHPTA. The effects of varying concentrations of the small molecules were evaluated. Fluorescence spectra and emission anisotropy were recorded on a Varian Cary Eclipse spectrometer. Protein concentration was determined by measuring the citrine absorbance at 515 nm using an extinction coefficient of 77000 M⁻¹cm⁻¹. The Citrine/Cerulean emission ration was calculated by dividing the emissions at 527 nm and 475 nm, respectively.

### Fluorescence Kd Measurement

The Atox, WD4 domain and hCCS (1 μM) were performed in 50 mM HEPES, 200 mM NaCl, 1 mM DTT (pH 7.1). Fluorescence spectra were excited at 278 nm and the maximum fluorescence emission at 310 nm (Tyr) and 330 nm (Trp), respectively. After treating with different concentrations of small molecules, the protein fluorescence emission was decreased and the emission of the small molecular was elevated. Following this fluorescence results, the Kds of the small molecules was calculated.

### Thermal Melt Shift Assay.

In brief, thermal shift assay of compound-protein interaction was performed in 384-well PCR plates with various compound concentrations and 200 μg/ml protein in a buffer solution (50 mM HEPES, 200 mM NaCl, 1 mM DTT, pH 7.4). SYPRO orange was used as a dye to monitor the fluorescence change at 610 nm. Small molecules were dissolved in DMSO and added to protein solution. Final DMSO concentration of solution is 1%.

### Live Cell Fluorescence Imaging

Hela cells were grown in DMEM media with 10% FBS and penicillin/streptomycin (Invitrogen). 24 hours after plating, the cells were transfected with pCDNA-YFP-Ace1 using Lipofectamine™ LTX transfection reagent (Invitrogen). 24 hours after transfection, cell was treated with Cu⁺ and compound 50, respectively. The acquisition of image data and synchronization of the illumination were performed on a fixed cell DSU spinning confocal microscope (Leica).

### Live Cell Time-Lapse Imaging

HeLa cells were maintained at 37° C. under 5% CO₂ in Dulbecco's modified Eagle's medium supplemented with 10% FBS. For the expression of pCDNA-YFP-Ace1, HeLa cells were plated onto Lab-Tek™ four-well-chambered coverglass at a density of 2×10⁵ cells/mL. After 24 hours, cells were transfected using Lipofectamine™ LTX transfection reagent (Invitrogen) according to the manufacturer's protocol. 24 hours after transfection, cell was treated with 150 μM Cu⁺ for 10 min then added 50 μM inhibitor 50 for 12 min. The acquisition of image data and synchronization of the illumination were performed on a fixed cell DSU spinning confocal microscope (Olympus). Images were collected every 2 min for 20 min (Cu⁺) and 2 min for 12 min (compound 50).

### Cancer Cell Culture

H1299, MDA-MB231, K562 cells were cultured in RPMI 1640 medium with 10% fetal bovine serum (FBS). 212LN cell lines were cultured in DMEM/Ham's F-12 50/50 mix medium in presence of 10% FBS 293T cells were cultured in Dulbecco Modified Eagle Medium (DMEM) with 10% FBS.

### Generate Stable Cell Lines.

Stable knockdown of endogenous Atox1 and hCCS were achieved using lentiviral vector harboring shRNA construct were purchased from Open Biosystems, Huntsville, Ala. Stable knockdown of overexpress Atox1 and hCCS were achieved using transfection by lipofctamine 2000 under selection G418.

### Cell Proliferation and Viability Assays.

For cell proliferation assay, 10×10⁴ cells were seeded in tissue culture coated 6-well plate and incubated at 37° C. for indicated times. Cell numbers were counted by trypan blue exclusion under a microscope (×40) at indicated times and the percentage of cell proliferation was determined by comparing Atox1 or hCCS knockdown cells to pLKO.1 vector expressing cells. For MTT cell viability assay of adherent cells, 5×10³ cells were seeded in 96-well plate 24 h before the assay starts and were cultured at 37° C. 24 h after seeding, cells were treated with compound 50 and incubated at 37° C. for 3 days. Cell viability was determined by using CellTiter96 Aqueous One solution proliferation kit (Promega).

### Xenograft Studies

Nude mice (nu/nu, male 6-8-week-old, Charles River Laboratories) were subcutaneously injected with 20×10⁶ H1299 cells or 10×10⁶ K562 cell on the right flanks. For drug evaluation of compound 50 using xenograft mice, the drug was administered by daily i.p. injection with a dose of 100 mg/kg from 6 days after subcutaneous injection of H1299 cells on right flank of each mouse. Tumor growth was recorded by measurement of two perpendicular diameters of the tumors over a 3-week course using the formula 4π/3×(width/2)²×(length/2). The tumors were harvested and weighed at the experimental endpoint, and the masses of tumors (g) treated with vehicle control (DMSO) and compound 50 was compared by a two-tailed unpaired Student's t test.

### Cell Proliferation and Viability Assays.

For cell proliferation assay, 10×10⁴ cells were seeded in tissue culture coated 6-well plate and incubated at 37° C. for indicated times. Cell numbers were counted by trypan blue exclusion under a microscope (×40) at indicated times and the percentage of cell proliferation was determined by comparing Atox1 or hCCS knockdown cells to pLKO.1 vector expressing cells. For MTT cell viability assay of adherent cells, 5×10³ cells were seeded in 96-well plate 24 h before the assay starts and were cultured at 37° C. 24 h after seeding, cells were treated with compound 50 and incubated at 37° C. for 3 days. Cell viability was determined by using CellTiter96 Aqueous One solution proliferation kit (Promega).

### Statistical Analysis

Statistical analysis and graphical presentation was done using GraphPad Prism 4.0. Data shown are from one representative experiment of multiple independent experiments and are given as mean±SD. Statistical analysis of significance (p values) was based on two-tailed Student's t test. The p value of the xenograft experiment was determined by the paired Student's t test.

### Measurement of ROS Production

Cells were treated with DMSO and DC_AC50 for 12 h and ROS generation was detected with DCFH-DA. Cells were incubated with 10 μM of DCFH-DA for 30 min at 37° C., washed twice with PBS, and immediately analyzed by a FACScan flow cytometer. The antioxidant NAC was treated with 3 mM concentration.

### Measurement of Intracellular ATP Production

An ATP bioluminescent somatic cell assay kit (Sigma) was used to measure intracellular ATP concentration. 1×10⁶ cells were trypsinized and resuspended in ultrapure water. Luminescence was measured with a spectrofluometer (SPECTRA Max Gemini; Molecular Probe) immediately after the addition of ATP enzyme mix to cell suspension.
¹⁴C-Lipids Synthesized from 14C-Glucose were Measured
Subconfluent cells were seeded on a 6-well plate. Cells were then incubated in complete medium spiked with 4 μCi/mL of D-[6-¹⁴C]-glucose for 2 h, washed twice with PBS, and lipids were extracted by the addition of 500 μL hexane:isopropanol (3:2 v/v). Wells were washed with an additional 500 μL of hexane:isopropanol solution, and extracts were combined and air dried with heat. Extracted lipids were resuspended in 50 μL of chloroform, and subjected to scintillation counting. Scintillation counts were normalized with cell numbers counted by a microscope (×40). ¹⁴C-RNA synthesized from ¹⁴C-glucose was measured. Subconfluent cells were seeded on a 6-well plate. Cells were then incubated in complete medium spiked with 4 μCi/mL of D-[U-¹⁴C]-glucose for 2. RNA was then extracted using RNeasy columns (Qiagen) and ¹⁴C-RNA was assayed by scintillation counter. ¹⁴C counts for each sample were normalized by the amount of RNA.

### Glucose Utilization Assays

1×10⁶ cells were plated onto a 6-cm dish one day prior to the assay. Media were replaced with phenol-red free RPMI with 1% FBS prior to continuous culture for 3 days. Medium samples were collected each day. Glucose concentrations in media were measured using a colormetric glucose assay kit (Biovision) and normalized with cell numbers.

### Lactate Production, Oxygen Consumption

Cellular lactate production was measured under normoxia with a fluorescence-based lactate assay kit (MBL). Phenol red-free RPMI medium without FBS was added to a 6 well-plate of subconfluent cells, and was incubated for 1 hour at 37° C. After incubation, 1 mL of media from each well was assessed using the lactate assay kit. A microscope (×40) was used to count cell numbers. Oxygen consumption rates were measured with a Clark-type electrode equipped with a 782 oxygen meter (Strathkelvin Instruments). 1×10⁷ cells were resuspended in RPMI 1640 medium with 10% FBS and placed inside a water-jacked chamber RC300 (Strathkelvin Instruments). Recording commenced immediately.

### Cell Cycle Arrest Assay

Cell-cycle arrest was assayed by using propidium iodide-stained (Muse™ Cell Cycle kit). 1×10⁶ cells were transferred to each tube and centrifuged at 300×g for 5 minutes then washed once with 1×PBS. The resuspended cells were slowly added 1 mL 70% ethanol to fix cells, which were incubated overnight at −20° C. The cells were centrifuged at 300×g for 5 minutes and ethanol was discarded. 200 μL of Muse cell cycle reagent was then added to each tube and incubated for 30 min at room temperature. Cell-cycle distributions were measured by flow cytometry.

### NADPH/NADP⁺ Ratio Assay

NADPH/NADP⁺ kit (BioAssay Systems) was used to measure the cellular NADPH/NADP⁺ ratio. Subconfluent cells seeded on a 10 cm dish were collected with a scraper, washed with PBS, and lysed with 200 μL of NADP⁺ (or NADPH) extraction buffer. Heat extract was allowed to proceed for 5 minutes at 60° C. before adding 20 μL of assay buffer and 200 μL of the counter NADPH (or NADP⁺) extraction buffer in order to neutralize the extracts. The extracts were spun down and the supernatants were reacted with the working buffer according to the manufacturer's protocol. The absorbance at 565 nm from the reaction mixture was measured with a plate reader.

### GSH/GSSG Ratio Assay

H1299 and K562 cells were grown in 96-well luminometer-compatible tissue culture plates. The GSH/GSSG ratio was determined using the GSH/GSSG-Glo assay (Promega) according to the manufacturer's protocol. Results are represented as mean and s.e.m of at least three independent experiments.

### SOD Activity Assay

Cells were grown until ˜80% confluent in 100-mm dishes (1×10⁷ cells). The cells were then washed three times with PBS, scraped from the dish, and pellet. The cell lysates were prepared in 50 mM Tris, pH 7.5, 150 mM NaCl, 1% Triton X-100, 10% glycerol and protease inhibitor cocktail (Sigma-Aldrich). The SOD activity was determined using the SOD determination kit (Sigma) according to the manufacturer's protocol. Results are represented as mean and s.e.m. of at least three independent experiments.

### Cytochrome c Oxidase Activity Assay

The Cytochrome c Oxidase activity was determined using the cytochrome c oxidase Assay kit (Sigma) according to the manufacturer's protocol. This kit is based on observation of the decrease in absorbance at 550 nm of ferrocytochrome c caused by its oxidation to ferricytochrome c by cytochrome c oxidase. Results are represented as mean and s.e.m. of at least three independent experiments.

### Antibodies

Antibodies used for immunoblotting were GAPDH (A00192; GenScript), Atox1 (sc-100557; santa cruz), CCS (sc-20141; santa cruz), SOD1 ((8B10); MA1-105; Thermo Scientific), SOD2 (PA5-30604; Thermo Scientific), Phospho-AMPKα ((40H9); 2535; Cell signaling), AMPKα (2532; Cell signaling), Phospho-ACC ((Ser79); 3661; Cell signaling), ACC ((C83B10); 3676; Cell signaling).

### Synthetic Procedures

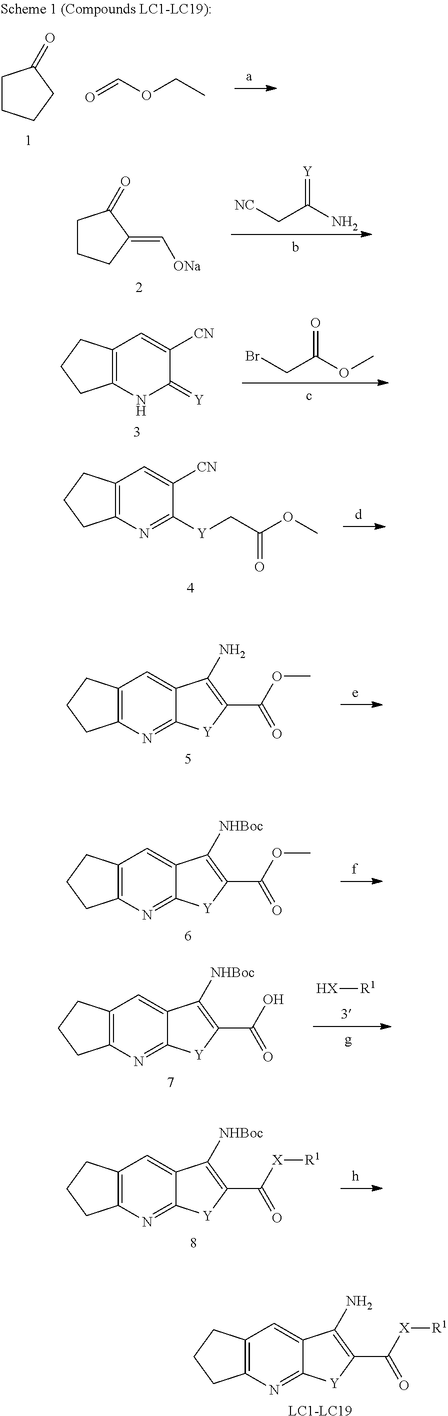

Experimental procedure for Scheme 1: Step a: To 1 equivalent of sodium metal in anhydrous diethyl ether is added 1-2 equivalents of ethyl formate and 1-2 equivalents of cyclopentanone. The resulting mixture is stirred overnight. The mother liquor is filtered by suction filtration to obtain crude intermediate 2.
Step b: To a solution of intermediate 2 in an organic solvent, is added 0.1 to 1 equivalent of glacial acetic acid. The reaction is stirred at 50-100° C., then 2′ and 0.1 to 1 equivalent of glacial acetic acid are added. The resulting reaction mixture is refluxed for 1-5 hours, filtered and recrystallized to produce product 3; the said organic solvent may optionally be tetrahydrofuran, ether, dimethylformamide, ethyleneglycol dimethyl ether, ethylene glycol diethyl ether, dioxane, ethanol, methanol, ethyl acetate, or dichloromethane.
Step c: To a solution of compound 3 in an organic solvent, is added 1 equivalent of methyl bromoacetate and an appropriate amount of base. The reaction mixture is stirted at room temperature to produce intermediate 4. The said organic solvent may optionally be tetrahydrofuran, aether, dimethylformamide, ethylene glycol dimethyl ether, ethylene glycol diethyl ether, dioxane, ethanol, methanol, ethyl acetate, or dichloromethane. The said base may optionally be potassium hydroxide, sodium hydroxide, sodium carbonate, potassium carbonate, cesium carbonate, and their aqueous solution in various concentrations.
Step d: The base described in Step c is added to a solution of compound 4 in an organic solvent. The reaction mixture is stirred and heated to produce intermediate 5.
Step e: An appropriate amount of di-tert-butyl dicarbonate and alkali are added to a solution of compound 5 in an organic solvent. The reaction is stirred to produce intermediate 6.
Step f: An appropriate amount of base is added to a solution of compound 6 in an organic solvent, which is then hydrolyzed to produce intermediate 7.
Step g: 3′ and a stoichiometric amount of condensing agent are added to a solution of compound 7 in an organic solvent. The reaction mixture is stirred until 3′ reacts completely to produce the final product. The said organic solvent may optionally be tetrahydrofuran, aether, dimethyl formamide, ethylene glycol dimethyl ether, ethylene glycol diethyl ether, dioxane, ethanol, methanol, ethyl acetate, or dichloromethane. The said condensing agent may optionally be DCC, EDCI, HOBt, and CDI.
Step h: To a solution of compound 7 in an organic solvent is added aqueous hydrochloric acid or trifluoroacetic acid. The reaction mixture is stirred vigorously to yield the BOC-deprotected final product.

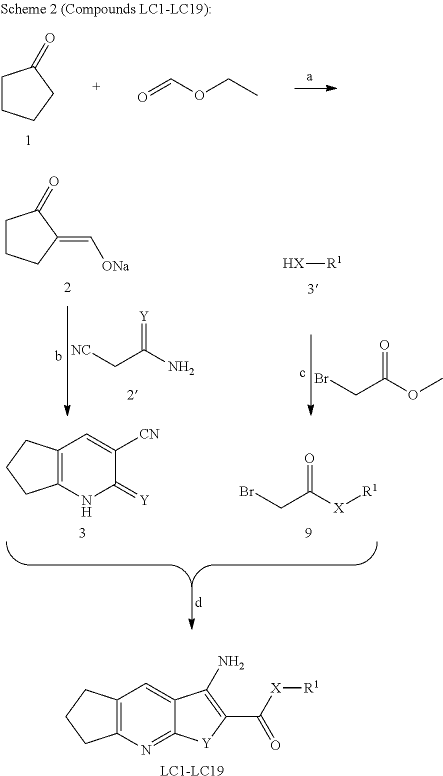

### Experimental Procedure for Scheme 2 (Compounds LC1-LC19):

Step a: Dissolve 1 equivalent of sodium in anhydrous ether, which shall be added slowly under an ice bath and rapid stirring condition. Add 1 equivalent of ethyl formate and 1 equivalent of cyclopentanone in a constant pressure dropping funnel, add 0.5 ml ethanol as an initiator, after 1 hour of stirring in ice bath, and stir overnight at room temperature until the reaction of sodium is finished. Perform suction filtration, wash with absolute ether to produce crude product for the following steps of reaction.
Step b: Dissolve the product in above steps directly in ethanol and control its amount, add an appropriate amount of glacial acetic acid, and stir and reflux under 70° C. Add cyano-sulfamide into the reaction solution, and add an appropriate amount of glacial acetic acid, react and reflux for about 3 hours. Recrystallize with ethanol to produce crude product.
Step c: Add 1 equivalent of the appropriate aniline or phenol and 2 equivalents of potassium carbonate solid in a round-bottomed flask that is placed in ice bath, add anhydrous THF to fully dissolve the solid, add 1.5 equivalents of bromoacetyl bromide into a constant pressure dropping funnel and dilute with THF, which is slowly dropped into the former said round-bottomed flask that is moved to room temperature in 10 min late and react for 1 hour; extract and dry with anhydrous sodium sulfate, filtrate by suction, and perform rotary evaporation to remove the solvent, and the crude product is obtained, which is to be used directly in the next step of reaction.
Step d: Dissolve the product from Step 2 into DMF under normal temperature by mixing, add 3 equivalents of 10% KOH solution, which is then transferred to an oil bath of 70° C. and react, and add I equivalent of the product from step 3. Stir for about 3 hours and then extract directly with ethyl acetate, and recrystallize the crude product with ethanol to produce pure end product.
Steps a and b: Intermediate 3 is prepared in accordance with the method outlined in Scheme 1.
Step c: 3′ and bromoacetyl bromide are condensed in the presence of a suitable base to produce intermediate 9. The said base may optionally be potassium hydroxide, sodium hydroxide, sodiumcarbonate, potassium carbonate, cesium carbonate, and their aqueous solution in various concentrations.
Step d: An appropriate amount of base is added to a solution of compound 3 in an organic solvent, and the reaction mixture is heated to 40-100° C. Intermediate 9 is added, and the heated solution is stirred for 1-10 hours to yield the final product. The said organic solvent may optionally be tetrahydrofuran, aether, dimethylformamide, ethylene glycol dimethyl ether, ethylene glycol diethyl ether, dioxane, ethanol, methanol. ethyl acetate, or dichloromethane. The said base may optionally be potassium hydroxide, sodium hydroxide, sodium carbonate, potassium carbonate, cesium carbonate, and their aqueous solution in various concentrations.

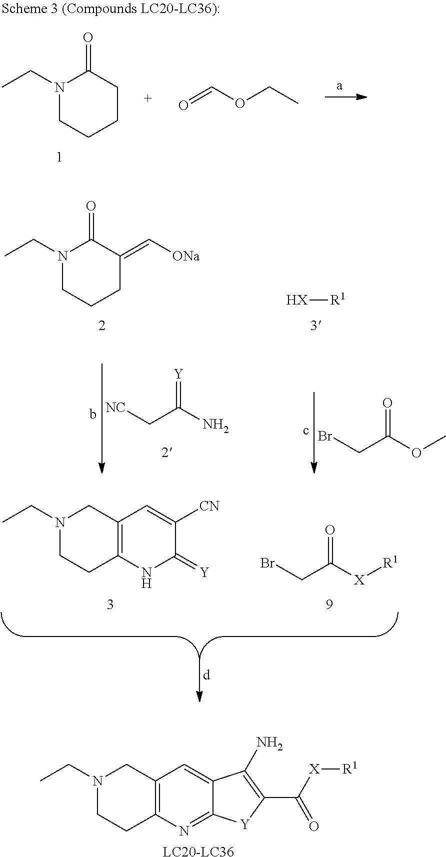

### Experimental Procedure for Scheme 3:

Step a: Dissolve 1 equivalent of sodium in anhydrous ether, which shall be added slowly under an ice bath and rapid stirring condition. Add 1 equivalent of ethyl formate and 1 equivalent of N-ethyl-piperidone in a constant pressure dropping funnel, add 0.5 ml ethanol as an initiator, after 1 hour of stirring in ice bath, and stir overnight at room temperature until the reaction of sodium is finished. Perform suction filtration, wash with absolute ether to produce crude product for the following steps of reaction.
Step b: Dissolve the product in above steps directly in ethanol and control its amount, add an appropriate amount of glacial acetic acid, and stir and reflux under 70° C. Add cyano-sulfamide into the reaction solution, and add an appropriate amount of glacial acetic acid, react and reflux for about 3 hours. Recrystallize with ethanol to produce crude product.
Step c: Add 1 equivalent of the appropriate aniline or phenol and 2 equivalents of potassium carbonate solid in a round-bottomed flask that is placed in ice bath, add anhydrous THF to fully dissolve the solid, add 1.5 equivalents of bromoacetyl bromide into a constant pressure dropping funnel and dilute with THF, which is slowly dropped into the former said round-bottomed flask that is moved to room temperature in 10 min late and react for 1 hour; extract and dry with anhydrous sodium sulfate, filtrate by suction, and perform rotary evaporation to remove the solvent, and the crude product is obtained, which is to be used directly in the next step of reaction.
Step d: Dissolve the product from Step 2 into DMF under normal temperature by mixing, add 3 equivalents of 10% KOH solution, which is then transferred to an oil bath of 70° C. and react, and add I equivalent of the product from step 3. Stir for about 3 hours and then extract directly with ethyl acetate, and recrystallize the crude product with ethanol to produce pure end product.

### Synthesis of Compounds LC37-LC39

Compound LC39 is synthesized in the same manner as the compounds in Scheme 3, except that N-ethyl-piperidone is replaced with N-acetyl piperidone. LC38, is synthesized in the same manner as the compounds in Scheme 3, except that N-ethyl-piperidone is replaced with N-Boc-piperidone. LC37 is synthesized by removing the Boc-protecting group of compound LC38 under acidic conditions.

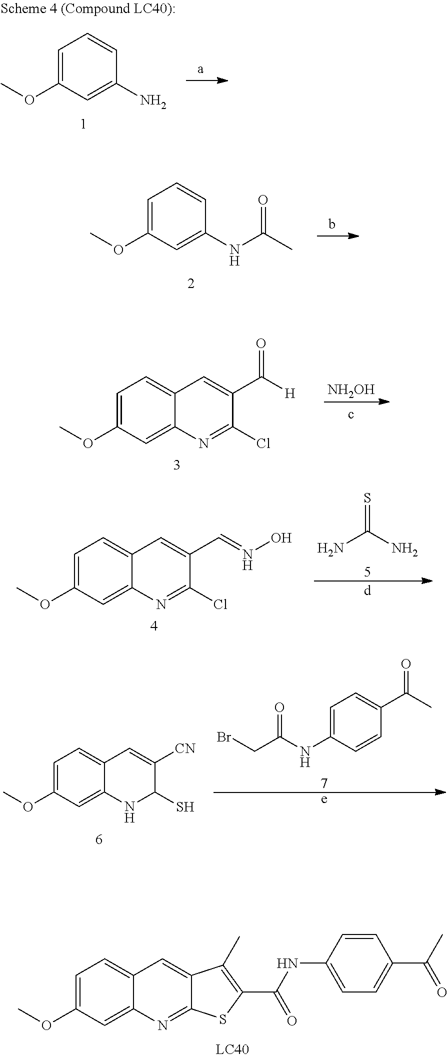

### Experimental Procedure for Scheme 4:

Step a: Prepare 2 by condensing starting material 1 with acetic anhydride;
Step b: Close ring in DMF to produce intermediate 3;
Step c: Prepare intermediate 4 by condensing intermediate 3 with hydroxylamine;
Step d: Prepare intermediate 6 by condensing intermediate 4 with raw materials;
Step e: Condense intermediate 6 with raw material 7 to produce the object compound LC-40.

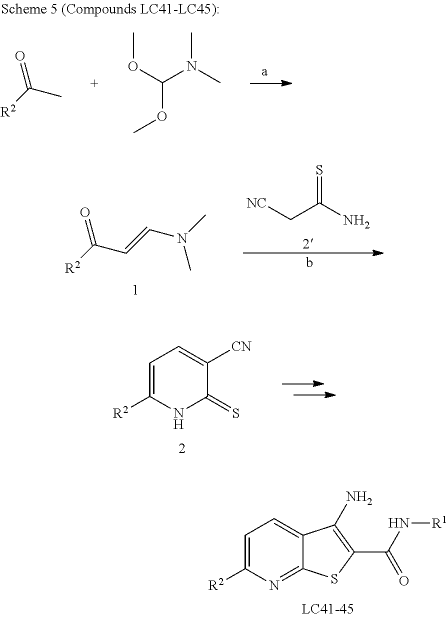

### Experimental Procedure for Scheme 5:

Step a: Prepare intermediate 1 by condensing various aromatic ethanones with N,N-dimethyl formamide dimethyl acetal.
Step b: Prepare intermediate by condensing intermediate 1 with cyanothiacetamide; then perform the subsequent operations similar to that in Scheme 2 to produce the final product.

### NMR and Mass Spectral Data:

### LC-1 (Compound 50)-3-amino-N-(2-bromo-4,6-difluorophenyl)-6,7-dihydro-5H-cyclopenta[b]thieno[3,2-e]pyridine-2-carboxamide

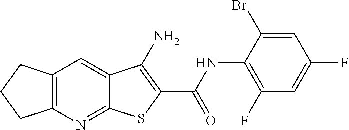 [SMILES: Nc1c(C(=O)Nc2c(F)cc(F)cc2Br)sc2nc3c(cc12)CCC3]

¹H NMR (CDCl₃, 400 MHz) δ 9.15 (s, 1H), 7.61 (s, 1H), 7.13 (m, 1H), 6.60 (m, 1H), 6.27 (s, 2H), 3.20 (t, 2H), 2.98 (t, 2H), 2.39 (m, 2H); ESI-MS (EI) m/z 422 (M⁺)

### LC-2-3-amino-N-phenyl-6,7-dihydro-5H-cyclopenta[b]thieno[3,2-e]pyridine-2-carboxamide

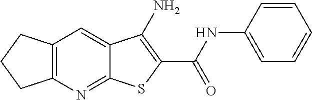 [SMILES: Nc1c(C(=O)Nc2ccccc2)sc2nc3c(cc12)CCC3]

¹H NMR (CDCl₃, 400 MHz) δ 9.15 (s, 1H), 7.61 (m, 3H), 7.43 (m, 2H), 7.19 (s, 1H), 6.27 (s, 2H), 3.20 (t, 2H), 2.98 (t, 2H), 2.39 (m, 2H); ESI-MS (EI) m/z 309 (M⁺)

### LC-3-3-amino-N-(2,4-difluorophenyl)-6,7-dihydro-5H-cyclopenta[b]thieno[3,2-e]pyridine-2-carboxamide

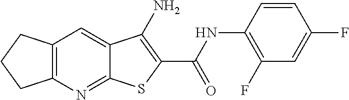 [SMILES: Nc1c(C(=O)Nc2ccc(F)cc2F)sc2nc3c(cc12)CCC3]

¹H NMR (CDCl₃, 400 MHz) δ 9.15 (s, 1H), 7.77 (m, 1H), 7.61 (s, 1H), 6.99 (m, 1H), 6.66 (m, 1H), 6.27 (s, 2H), 3.20 (t, 2H), 2.98 (t, 2H), 2.39 (m, 2H); ESI-MS (EI) m/z 345 (M⁺)

### LC-4-3-amino-N-(2-fluorophenyl)-6,7-dihydro-5H-cyclopenta[b]thieno[3,2-e]pyridine-2-carboxamide

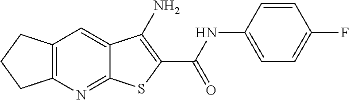 [SMILES: Nc1c(C(=O)Nc2ccc(F)cc2)sc2nc3c(cc12)CCC3]

¹H NMR (CDCl₃, 400 MHz) δ 9.15 (s, 1H), 7.61 (s, 1H), 7.60 (m, 2H), 7.22 (m, 2H), 6.27 (s, 2H), 3.20 (t, 2H), 2.98 (t, 2H), 2.39 (m, 2H); ESI-MS (EI) m/z 323 (M⁺)

### LC-5-3-amino-N-p-tolyl-6,7-dihydro-5H-cyclopenta[b]thieno[3,2-e]pyridine-2-carboxamide

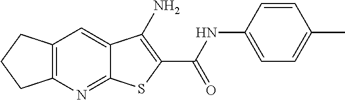 [SMILES: Cc1ccc(NC(=O)c2sc3nc4c(cc3c2N)CCC4)cc1]

¹H NMR (CDCl₃, 400 MHz) δ 9.15 (s, 1H), 7.61 (s, 1H), 7.56 (m, 2H), 7.21 (m, 2H), 6.27 (s, 2H), 3.20 (t, 2H), 2.98 (t, 2H), 2.39 (m, 2H), 2.39 (s, 3H); ESI-MS (EI) m/z 323 (M⁺)

### LC-6-3-amino-N-(4-bromophenyl)-6,7-dihydro-5H-cyclopenta[b]thieno[3,2-e]pyridine-2-carboxamide

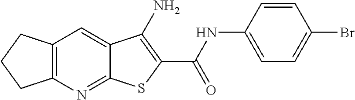 [SMILES: Nc1c(C(=O)Nc2ccc(Br)cc2)sc2nc3c(cc12)CCC3]

¹H NMR (CDCl₃, 400 MHz) δ 9.15 (s, 1H), 7.70 (m, 2H), 7.61 (m, 1H), 7.58 (m, 2H), 6.27 (s, 2H), 3.20 (t, 2H), 2.98 (t, 2H), 2.39 (m, 2H); ESI-MS (EI) m/z 387 (M⁺)

### LC-7-3-amino-N-(4-(trifluoromethoxy)phenyl)-6,7-dihydro-5H-cyclopenta[b]thieno[3,2-e]pyridine-2-carboxamide

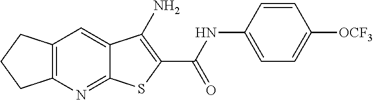 [SMILES: Nc1c(C(=O)Nc2ccc(OC(F)(F)F)cc2)sc2nc3c(cc12)CCC3]

¹H NMR (CDCl₃, 400 MHz) δ 9.15 (s, 1H), 7.61 (s, 1H), 7.51 (m, 2H), 6.97 (m, 2H), 6.27 (s, 2H), 3.20 (t, 2H), 2.98 (t, 2H), 2.39 (m, 2H); ESI-MS (EI) m/z 393 (M⁺)

### LC-8-3-amino-N-(4-cyanophenyl)-6,7-dihydro-5H-cyclopenta[b]thieno[3,2-e]pyridine-2-carboxamide

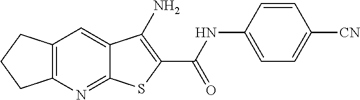 [SMILES: N#Cc1ccc(NC(=O)c2sc3nc4c(cc3c2N)CCC4)cc1]

¹H NMR (CDCl₃, 400 MHz) δ 9.15 (s, 1H), 7.99 (m, 2H), 7.62 (m, 2H), 7.61 (s, 1H), 6.27 (s, 2H), 3.20 (t, 2H), 2.98 (t, 2H), 2.39 (m, 2H); ESI-MS (EI) m/z 334 (M⁺)

### LC-9-3-amino-N-(2-bromophenyl)-6,7-dihydro-5H-cyclopenta[b]thieno[3,2-e]pyridine-2-carboxamide

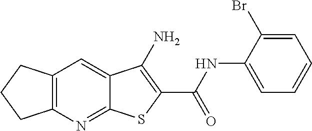 [SMILES: Nc1c(C(=O)Nc2ccccc2Br)sc2nc3c(cc12)CCC3]

¹H NMR (CDCl₃, 400 MHz) δ 9.15 (s, 1H), 8.06 (m, 1H), 7.78 (m, 1H), 7.61 (s, 1H), 7.37 (m, 1H), 7.08 (m, 1H), 6.27 (s, 2H), 3.20 (t, 2H), 2.98 (t, 2H), 2.39 (m, 2H); ESI-MS (EI) m/z 387 (M⁺)

### LC-10-3-amino-N-(2-fluorophenyl)-6,7-dihydro-5H-cyclopenta[b]thieno[3,2-e]pyridine-2-carboxamide

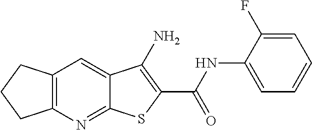 [SMILES: Nc1c(C(=O)Nc2ccccc2F)sc2nc3c(cc12)CCC3]

¹H NMR (CDCl₃, 400 MHz) δ 9.15 (s, 1H), 7.96 (m, 1H), 7.61 (s, 1H), 7.22 (m, 1H), 7.21 (m, 1H), 7.01 (m, 1H), 6.27 (s, 2H), 3.20 (t, 2H), 2.98 (t, 2H), 2.39 (m, 2H); ESI-MS (EI) m/z 327 (M⁺)

### LC-11-3-amino-N-(2-(trifluoromethoxy)phenyl)-6,7-dihydro-5H-cyclopenta-[b]thieno[3,2-e]pyridine-2-carboxamide

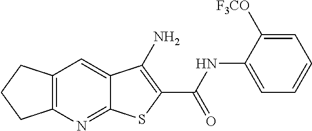 [SMILES: Nc1c(C(=O)Nc2ccccc2OC(F)(F)F)sc2nc3c(cc12)CCC3]

¹H NMR (CDCl₃, 400 MHz) δ 9.15 (s, 1H), 7.85 (m, 1H), 7.61 (s, 1H), 7.08 (m, 2H), 6.99 (m, 1H), 6.27 (s, 2H), 3.20 (t, 2H), 2.98 (t, 2H), 2.39 (m, 2H); ESI-MS (EI) m/z 393 (M⁺)

### LC-12-3-amino-N-(2-(trifluoromethyl)phenyl)-6,7-dihydro-5H-cyclopenta[b]thieno[3,2-e]pyridine-2-carboxamide

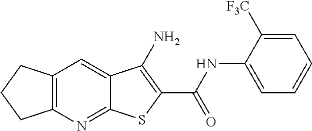 [SMILES: Nc1c(C(=O)Nc2ccccc2C(F)(F)F)sc2nc3c(cc12)CCC3]

¹H NMR (CDCl₃, 400 MHz) δ 9.15 (s, 1H), 7.71 (m, 1H), 7.61 (s, 1H), 7.46 (m, 1H), 7.43 (m, 1H), 7.35 (m, 1H), 6.27 (s, 2H), 3.20 (t, 2H), 2.98 (t, 2H), 2.39 (m, 2H); ESI-MS (EI) m/z 377 (M⁺)

### LC-13-3-amino-N-(2-nitrophenyl)-6,7-dihydro-5H-cyclopenta[b]thieno[3,2-e]pyridine-2-carboxamide

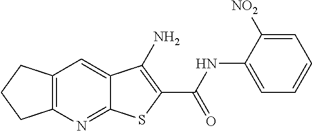 [SMILES: Nc1c(C(=O)Nc2ccccc2[N+](=O)[O-])sc2nc3c(cc12)CCC3]

¹H NMR (CDCl₃, 400 MHz) δ 9.15 (s, 1H), 8.53 (m, IH), 8.24 (m, 1H), 7.82 (m, 1H), 7.78 (m, 1H), 7.61 (s, 1H), 6.27 (s, 2H), 3.20 (t, 2H), 2.98 (t, 2H), 2.39 (m, 2H); ESI-MS (EI) m/z 354 (M⁺)

### LC-14-3-amino-N-(pyridin-2-yl)-6,7-dihydro-5H-cyclopenta[b]thieno[3,2-e]pyridine-2-carboxamide

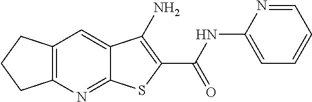 [SMILES: Nc1c(C(=O)Nc2ccccn2)sc2nc3c(cc12)CCC3]

¹H NMR (CDCl₃, 400 MHz) δ 9.15 (s, 1H), 8.07 (m, 1H), 7.61 (s, 1H), 7.55 (m, 1H), 6.62 (m, 1H), 6.53 (m, 1H), 6.27 (s, 2H), 3.20 (t, 2H), 2.98 (t, 2H), 2.39 (m, 2H); ESI-MS (EI) m/z 310 (M⁺)

### LC-15-3-amino-N-(naphthalen-2-yl)-6,7-dihydro-5H-cyclopenta[b]thieno[3,2-e]pyridine-2-carboxamide

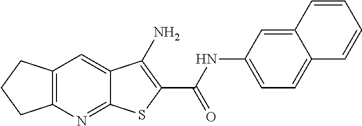 [SMILES: Nc1c(C(=O)Nc2ccc3ccccc3c2)sc2nc3c(cc12)CCC3]

¹H NMR (CDCl₃, 400 MHz) δ 9.15 (s, 1H), 8.29 (s, 1H), 7.88 (m, 1H), 7.84 (m, 1H), 7.77 (m, 1H), 7.61 (s, 1H), 7.50 (m, 1H), 7.49 (m, 1H), 7.36 (m, 1H), 6.27 (s, 2H), 3.20 (t, 2H), 2.98 (t, 2H), 2.39 (m, 2H); ESI-MS (EI) m/z 359 (M⁺)

### LC-16-3-amino-N-(2-bromo-4,6-difluorophenyl)-6,7-dihydro-5H-cyclopenta[b]furo[3,2-e]pyridine-2-carboxamide

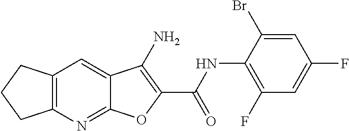 [SMILES: Nc1c(C(=O)Nc2c(F)cc(F)cc2Br)oc2nc3c(cc12)CCC3]

¹H NMR (CDCl₃, 400 MHz) δ 9.15 (s, 1H), 7.61 (s, 1H), 7.13 (m, 1H), 6.60 (m, 1H), 6.27 (s, 2H), 3.20 (t, 2H), 2.98 (t, 2H), 2.39 (m, 2H); ESI-MS (EI) m/z 407 (M⁺)

### LC-17-3-amino-N-(2-(trifluoromethoxy)phenyl)-6,7-dihydro-5H-cyclopenta[b]furo[3,2-e]pyridine-2-carboxamide

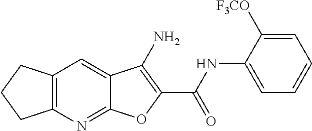 [SMILES: Nc1c(C(=O)Nc2ccccc2OC(F)(F)F)oc2nc3c(cc12)CCC3]

¹H NMR (CDCl₃, 400 MHz) δ 9.15 (s, 1H), 7.85 (m, 1H), 7.61 (s, 1H), 7.08 (m, 2H), 6.99 (m, 1H), 6.27 (s, 2H), 3.20 (t, 2H), 2.98 (t, 2H), 2.39 (m, 2H); ESI-MS (EI) m/z 377 (M⁺)

### LC-18-2-bromo-4,6-difluorophenyl 3-amino-6,7-dihydro-5H-cyclopenta[b]thieno[3,2-e]pyridine-2-carboxylate

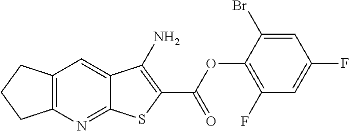 [SMILES: Nc1c(C(=O)Oc2c(F)cc(F)cc2Br)sc2nc3c(cc12)CCC3]

¹H NMR (CDCl₃, 400 MHz) δ 9.15 (s, 1H), 7.12 (m, 1H), 6.59 (m, 1H), 6.27 (s, 2H), 3.20 (t, 2H), 2.98 (t, 2H), 2.39 (m, 2H); ESI-MS (EI) m/z 423 (M⁺)

### LC-19-2-(trifluoromethoxy)phenyl 3-amino-6,7-dihydro-5H-cyclopenta[b]thieno[3,2-e]pyridine-2-carboxylate

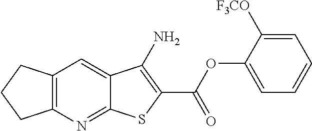 [SMILES: Nc1c(C(=O)Oc2ccccc2OC(F)(F)F)sc2nc3c(cc12)CCC3]

¹H NMR (CDCl₃, 400 MHz) δ 7.61 (s, 1H), 7.18 (m, 1H), 7.15 (m, 1H), 6.98 (m, 1H), 6.96 (m, 1H), 6.27 (s, 2H), 3.20 (t, 2H), 2.98 (t, 2H), 2.39 (m, 2H); ESI-MS (EI) m/z 394 (M⁺)

### LC-20-3-amino-6-ethyl-N-phenyl-5,6,7,8-tetrahydrothieno[2,3-b][1,6]-naphthyridine-2-carboxamide

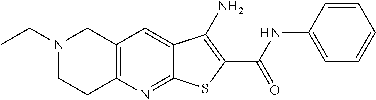 [SMILES: CCN1CCc2nc3sc(C(=O)Nc4ccccc4)c(N)c3cc2C1]

¹H NMR (CDCl₃, 400 MHz) δ 9.15 (s, 1H), 7.80 (s, 1H), 7.61 (m, 2H), 7.43 (m, 2H), 7.19 (m, 1H), 6.27 (s, 2H), 3.70 (s, 2H), 3.09 (t, 2H), 2.85 (m, 2H), 2.78 (m, 2H), 1.02 (t, 3H); ESI-MS (EI) m/z 352 (M⁺)

### LC-21 (Compound 2)-3-amino-6-ethyl-N-(2-(trifluoromethoxy)phenyl)-5,6,7,8-tetrahydro-thieno[2,3-b][1,6]naphthyridine-2-carboxamide

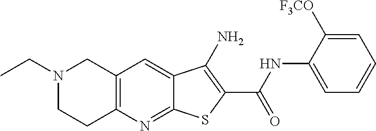 [SMILES: CCN1CCc2nc3sc(C(=O)Nc4ccccc4OC(F)(F)F)c(N)c3cc2C1]

¹H NMR (CDCl₃, 400 MHz) δ 9.15 (s, 1H), 7.85 (m, 1H), 7.80 (s, 1H), 7.08 (m, 2H), 6.99 (m, 1H), 6.27 (s, 2H), 3.70 (s, 2H), 3.09 (t, 2H), 2.85 (m, 2H), 2.78 (m, 2H), 1.02 (t, 3H); ESI-MS (EI) m/z 436 (M⁺)

### LC-22-3-amino-N-(2-bromo-4,6-difluorophenyl)-6-ethyl-5,6,7,8-tetrahydro-thieno[2,3-b][1,6]naphthyridine-2-carboxamide

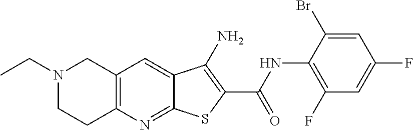 [SMILES: CCN1CCc2nc3sc(C(=O)Nc4c(F)cc(F)cc4Br)c(N)c3cc2C1]

¹H NMR (CDCl₃, 400 MHz) δ 9.15 (s, 1H), 7.80 (s, 1H), 7.13 (d, 1H), 6.60 (t, 1H), 6.27 (s, 2H), 3.70 (s, 2H), 3.09 (t, 2H), 2.85 (m, 2H), 2.78 (m, 2H), 1.02 (t, 3H); ESI-MS (EI) m/z 466 (M⁺)

### LC-23-3-amino-N-(2,4-difluorophenyl)-6-ethyl-5,6,7,8-tetrahydrothieno[2,3-b][1,6]naphthyridine-2-carboxamide

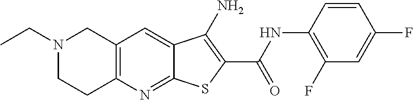 [SMILES: CCN1CCc2nc3sc(C(=O)Nc4ccc(F)cc4F)c(N)c3cc2C1]

¹H NMR (CDCl₃, 400 MHz) δ 9.15 (s, 1H), 7.80 (s, 1H), 7.77 (m, 1H), 6.99 (m, 1H), 6.66 (t, 1H), 6.27 (s, 2H), 3.70 (s, 2H), 3.09 (t, 2H), 2.85 (m, 2H), 2.78 (m, 2H), 1.02 (t, 3H); ESI-MS (EI) m/z 388 (M⁺)

### LC-24-3-amino-6-ethyl-N-(2-fluorophenyl)-5,6,7,8-tetrahydrothieno[2,3-b][1,6]naphthyridine-2-carboxamide

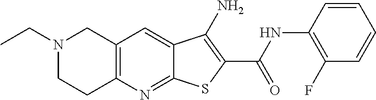 [SMILES: CCN1CCc2nc3sc(C(=O)Nc4ccccc4F)c(N)c3cc2C1]

¹H NMR (CDCl₃, 400 MHz) δ 9.15 (s, 1H), 7.96 (m, 1H), 7.80 (s, 1H), 7.22 (m, 1H), 7.20 (m, 1H), 7.01 (t, 1H), 6.27 (s, 2H), 3.70 (s, 2H), 3.09 (t, 2H), 2.85 (m, 2H), 2.78 (m, 2H), 1.02 (t, 3H); ESI-MS (EI) mlz 370 (M⁺)

### LC-25-3-amino-N-(2-cyanophenyl)-6-ethyl-5,6,7,8-tetrahydrothieno[2,3-b][1,6]naphthyridine-2-carboxamide

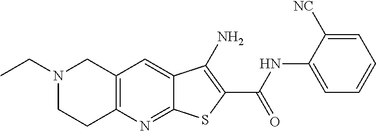 [SMILES: CCN1CCc2nc3sc(C(=O)Nc4ccccc4C#N)c(N)c3cc2C1]

¹H NMR (CDCl₃, 400 MHz) δ 9.15 (s, 1H), 7.99 (m, 1H), 7.80 (s, 1H), 7.71 (m, 1H), 7.62 (m, 1H), 7.37 (m, 1H), 6.27 (s, 2H), 3.70 (s, 2H), 3.09 (t, 2H), 2.85 (m, 2H), 2.78 (m, 2H), 1.02 (t, 3H); ESI-MS (EI) m/z 377 (M⁺)

### LC-26-3-amino-6-ethyl-N-o-tolyl-5,6,7,8-tetrahydrothieno[2,3-b][1,6]naphthyridine-2-carboxamide

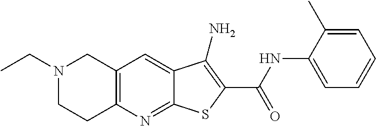 [SMILES: CCN1CCc2nc3sc(C(=O)Nc4ccccc4C)c(N)c3cc2C1]

¹H NMR (CDCl₃, 400 MHz) δ 9.15 (s, 1H), 7.80 (s, 1H), 7.38 (m, 1H), 7.30 (m, 1H), 7.24 (m, 1H), 7.07 (m, 1H), 6.27 (s, 2H), 3.70 (s, 2H), 3.09 (t, 2H), 2.85 (m, 2H), 2.78 (m, 2H), 2.12 (s, 3H), 1.02 (t, 3H); ESI-MS (EI) m/z 366 (M⁺)

### LC-27-3-amino-6-ethyl-N-(2-methoxyphenyl)-5,6,7,8-tetrahydrothieno[2,3-b][1,6]naphthyridine-2-carboxamide

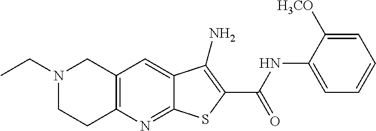 [SMILES: CCN1CCc2nc3sc(C(=O)Nc4ccccc4OC)c(N)c3cc2C1]

¹H NMR (CDCl₃, 400 MHz) δ 9.15 (s, 1H), 7.85 (m, 1H), 7.80 (s, 1H), 7.08 (m, 2H), 6.99 (m, 1H), 6.27 (s, 2H), 3.83 (s, 3H), 3.70 (s, 2H), 3.09 (t, 2H), 2.85 (m, 2H), 2.78 (m, 2H), 1.02 (t, 3H); ESI-MS (EI) m/z 382 (M⁺)

### LC-28-3-amino-6-ethyl-N-(4-nitrophenyl)-5,6,7,8-tetrahydrothieno[2,3-b][1,6]naphthyridine-2-carboxamide

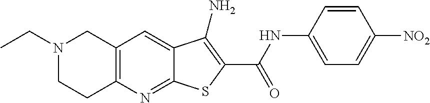 [SMILES: CCN1CCc2nc3sc(C(=O)Nc4ccc([N+](=O)[O-])cc4)c(N)c3cc2C1]

¹H NMR (CDCl₃, 400 MHz) δ 9.15 (s, 1H), 8.24 (m, 2H), 7.82 (m, 2H), 7.80 (s, 1H), 6.27 (s, 2H), 3.70 (s, 2H), 3.09 (t, 2H), 2.85 (m, 2H), 2.78 (m, 2H), 1.02 (t, 3H); ESI-MS (EI) m/z 397 (M⁺)

### LC-29-3-amino-N-(4-aminophenyl)-6-ethyl-5,6,7,8-tetrahydrothieno[2,3-b][1,6]naphthyridine-2-carboxamide

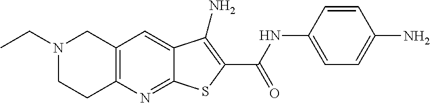 [SMILES: CCN1CCc2nc3sc(C(=O)Nc4ccc(N)cc4)c(N)c3cc2C1]

¹H NMR (CDCl₃, 400 MHz) δ 9.15 (s, 1H), 7.80 (s, 1H), 7.39 (m, 2H), 6.61 (m, 2H), 6.27 (s, 4H), 3.70 (s, 2H), 3.09 (t, 2H), 2.85 (m, 2H), 2.78 (m, 2H), 1.02 (t, 3H); ESI-MS (EI) m/z 367 (M⁺)

### LC-30-3-amino-6-ethyl-N-(pyridin-2-yl)-5,6,7,8-tetrahydrothieno[2,3-b][1,6]naphthyridine-2-carboxamide

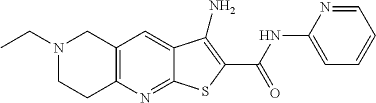 [SMILES: CCN1CCc2nc3sc(C(=O)Nc4ccccn4)c(N)c3cc2C1]

¹H NMR (CDCl₃, 400 MHz) δ 9.15 (s, 1H), 8.07 (m, 1H), 7.80 (s, 1H), 7.55 (m, 1H), 6.62 (m, 1H), 6.53 (m, 1H), 6.27 (s, 4H), 3.70 (s, 2H), 3.09 (t, 2H), 2.85 (m, 2H), 2.78 (m, 2H), 1.02 (t, 3H); ESI-MS (EI) m/z 353 (M⁺)

### LC-31-3-amino-6-ethyl-N-(naphthalen-2-yl)-5,6,7,8-tetrahydrothieno[2,3-b][1,6]naphthyridine-2-carboxamide

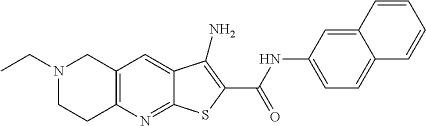 [SMILES: CCN1CCc2nc3sc(C(=O)Nc4ccc5ccccc5c4)c(N)c3cc2C1]

¹H NMR (CDCl₃, 400 MHz) δ 9.15 (s, 1H), 8.29 (s, 1H), 7.88 (m, 1H), 7.84 (m, 1H), 7.80 (s, 1H), 7.77 (m, 1H), 7.50 (m, 1H), 7.49 (m, 1H), 7.36 (m, 1H), 6.27 (s, 2H), 3.70 (s, 2H), 3.09 (t, 2H), 2.85 (m, 2H), 2.78 (m, 2H), 1.02 (t, 3H); ESI-MS (EI) m/z 402 (M⁺)

### LC-32-3-amino-N-(biphenyl-2-yl)-6-ethyl-5,6,7,8-tetrahydrothieno[2,3-b][1,6]naphthyridine-2-carboxamide

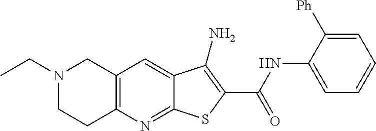 [SMILES: CCN1CCc2nc3sc(C(=O)Nc4ccccc4-c4ccccc4)c(N)c3cc2C1]

¹H NMR (CDCl₃, 400 MHz) δ 9.15 (s, 1H), 7.80 (s, 1H), 7.77 (m, 1H), 7.51 (m, 2H), 7.49 (m, 1H), 7.41 (m, 1H), 7.39 (m, 1H), 7.25 (m, 1H), 7.08 (m, 2H), 6.27 (s, 2H), 3.70 (s, 2H), 3.09 (t, 2H), 2.85 (m, 2H), 2.78 (m, 2H), 1.02 (t, 3H); ESI-MS (EI) m/z 428 (M⁺)

### LC-33-3-amino-6-ethyl-N-(2-(trifluoromethoxy)phenyl)-5,6,7,8-tetrahydrofuro[2,3-b][1,6]naphthyridine-2-carboxamide

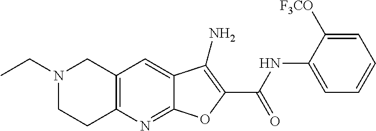 [SMILES: CCN1CCc2nc3oc(C(=O)Nc4ccccc4OC(F)(F)F)c(N)c3cc2C1]

¹H NMR (CDCl₃, 400 MHz) δ 9.15 (s, 1H), 7.85 (m, 1H), 7.80 (s, IH), 7.08 (m, 2H), 6.99 (m, IH), 6.27 (s, 2H), 3.70 (s, 2H), 3.09 (t, 2H), 2.85 (m, 2H), 2.78 (m, 2H), 1.02 (t, 3H); ESI-MS (EI) m/z 420 (M⁺)

### LC-34-3-amino-N-(2-bromo-4,6-difluorophenyl)-6-ethyl-5,6,7,8-tetrahydrofuro[2,3-b][1,6]naphthyridine-2-carboxamide

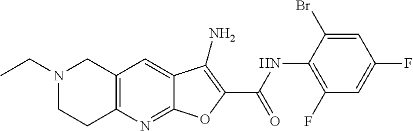 [SMILES: CCN1CCc2nc3oc(C(=O)Nc4c(F)cc(F)cc4Br)c(N)c3cc2C1]

¹H NMR (CDCl₃, 400 MHz) δ 9.15 (s, 1H), 7.80 (s, 1H), 7.13 (m, 1H), 6.60 (m, 1H), 6.27 (s, 2H), 3.70 (s, 2H), 3.09 (t, 2H), 2.85 (m, 2H), 2.78 (m, 2H), 1.02 (t, 3H); ESI-MS (EI) m/z 450 (M⁺)

### LC-35-2-(trifluoromethoxy)phenyl 3-amino-6-ethyl-5,6,7,8-tetrahydrothieno[2,3-b][1,6]naphthyridine-2-carboxylate

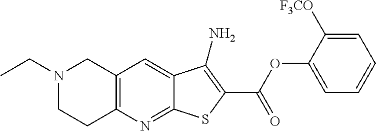 [SMILES: CCN1CCc2nc3sc(C(=O)Oc4ccccc4OC(F)(F)F)c(N)c3cc2C1]

¹H NMR (CDCl₃, 400 MHz) δ 7.80 (s, 1H), 7.18 (m, 1H), 7.15 (m, 1H), 6.98 (m, 1H), 6.96 (m, 1H), 6.27 (s, 2H), 3.70 (s, 2H), 3.09 (t, 2H), 2.85 (m, 2H), 2.78 (m, 2H), 1.02 (t, 3H); ESI-MS (EI) m/z 437 (M⁺)

### LC-36-2-bromo-4,6-difluorophenyl 3-amino-6-ethyl-5,6,7,8-tetrahydrothieno[2,3-b][1,6]naphthyridine-2-carboxylate

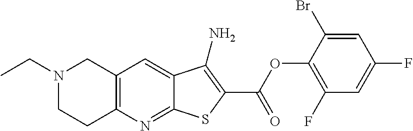 [SMILES: CCN1CCc2nc3sc(C(=O)Oc4c(F)cc(F)cc4Br)c(N)c3cc2C1]

¹H NMR (CDCl₃, 400 MHz) δ 7.80 (s, 1H), 7.12 (m, 1H), 6.59 (m, 1H), 6.27 (s, 2H), 3.70 (s, 2H), 3.09 (t, 2H), 2.85 (m, 2H), 2.78 (m, 2H), 1.02 (t, 3H); ESI-MS (EI) m/z 467 (M⁺)

### LC-37-3-amino-N-(2-(trifluoromethoxy)phenyl)-5,6,7,8-tetrahydrothieno[2,3-b][1,6]naphthyridine-2-carboxamide

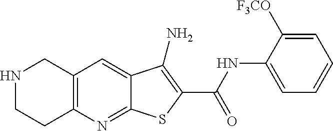 [SMILES: Nc1c(C(=O)Nc2ccccc2OC(F)(F)F)sc2nc3c(cc12)CNCC3]

¹H NMR (CDCl₃, 400 MHz) δ 9.15-7.85 (m, 1H), 7.80 (s, 1H), 7.08 (m, 2H), 6.99 (m, 1H), 6.27 (s, 2H), 3.81 (s, 2H), 3.36 (t, 2H), 3.11 (m, 2H), 1.91 (s, 1H); ESI-MS (EI) m/z 408 (s, 1H), (M⁺)

### LC-38-tert-butyl 3-amino-2-(2-(trifluoromethoxy)phenylcarbamoyl)-7,8-dihydrothieno[2,3-b][1,6]naphthyridine-6(5H)-carboxylate

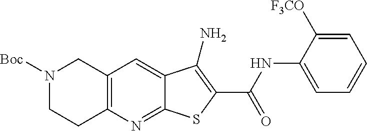 [SMILES: CC(C)(C)OC(=O)N1CCc2nc3sc(C(=O)Nc4ccccc4OC(F)(F)F)c(N)c3cc2C1]

¹H NMR (CDCl₃, 400 MHz) δ 9.15 (s, 1H), 7.85 (m, 1H), 7.80 (s, 1H), 7.08 (m, 2H), 6.99 (m, 1H), 6.27 (s, 2H), 4.22 (s, 2H), 3.60 (t, 2H), 3.25 (m, 2H), 1.38 (s, 9H); ESI-MS (EI) m/z 508 (M⁺)

### LC-39-6-acetyl-3-amino-N-(2-(trifluoromethoxy)phenyl)-5,6,7,8-tetrahydrothieno[2,3-b][1,6]naphthyridine-2-carboxamide

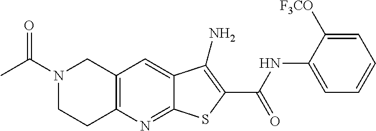 [SMILES: CC(=O)N1CCc2nc3sc(C(=O)Nc4ccccc4OC(F)(F)F)c(N)c3cc2C1]

¹H NMR (CDCl₃, 400 MHz) δ 9.15 (s, 1H), 7.85 (m, 1H), 7.80 (s, 1H), 7.08 (m, 2H), 6.99 (m, 1H), 6.27 (s, 2H), 4.46 (s, 2H), 3.84 (t, 2H), 3.25 (m, 2H), 2.32 (s, 3H); ESI-MS (EI) m/z 450 (M⁺)

### LC-40 (Compound 61)-N-(4-acetylphenyl)-3-amino-7-methoxythieno[2,3-b]quinoline-2-carboxamide

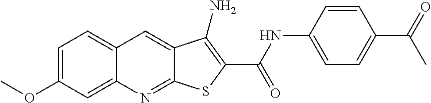 [SMILES: COc1ccc2cc3c(N)c(C(=O)Nc4ccc(C(C)=O)cc4)sc3nc2c1]

¹H NMR (CDCl₃, 400 MHz) δ 9.15 (s, 1H), 7.99 (m, 1H), 7.97 (d, 1H), 7.92 (m, 2H), 7.77 (d, 2H), 7.33 (s, 1H), 7.25 (m, 1H), 6.27 (s, 2H), 3.83 (s, 3H), 2.50 (s, 3H); ESI-MS (EI) m/z 391 (M⁺)

### LC-41-3-amino-N-(3,4-dimethoxyphenyl)-6-(4-fluorophenyl)thieno[2,3-b]pyridine-2-carboxamide

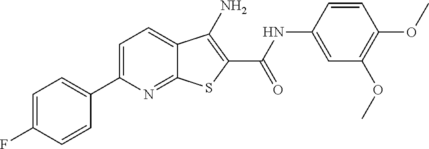 [SMILES: COc1ccc(NC(=O)c2sc3nc(-c4ccc(F)cc4)ccc3c2N)cc1OC]

¹H NMR (CDCl₃, 400 MHz) δ 9.15 (s, 1H), 8.66 (m, 2H), 7.97 (d, 1H), 7.47 (d, 1H), 7.33 (m, 2H), 7.25 (m, 1H), 7.07 (m, 1H), 6.86 (m, 1H), 6.27 (s, 2H), 3.83 (s, 6H); ESI-MS (EI) m/z 42 (M⁺)

### LC-42 (Compound 49)-3-amino-N-(2-chloro-5-methoxyphenyl)-6-(3-methoxyphenyl)thieno[2,3-b]pyridine-2-carboxamide

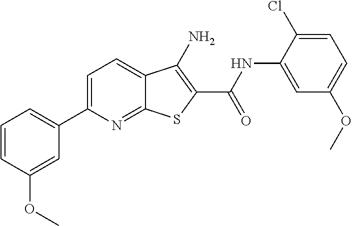 [SMILES: COc1cccc(-c2ccc3c(N)c(C(=O)Nc4cc(OC)ccc4Cl)sc3n2)c1]

¹H NMR (CDCl₃, 400 MHz) δ 9.15 (s, 1H), 7.97 (m, 1H), 7.87 (d, 1H), 7.86 (d, 1H), 7.47 (m, 1H), 7.43 (m, 1H), 7.36 (m, 1H), 7.30 (m, 1H), 7.01 (m, 1H), 6.77 (m, 1H), 6.27 (s, 2H), 3.83 (s, 6H); ESI-MS (EI) m/z 439 (M⁺)

### LC-43-3-amino-N-(2,5-dimethoxyphenyl)-6-(thiophen-2-yl)thieno[2,3-b]pyridine-2-carboxamide

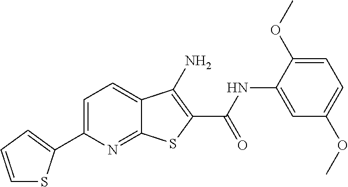 [SMILES: COc1ccc(OC)c(NC(=O)c2sc3nc(-c4cccs4)ccc3c2N)c1]

¹H NMR (CDCl₃, 400 MHz) δ 9.15 (s, 1H), 8.03 (m, 1H), 7.85 (d, 1H), 7.81 (d, 1H), 7.69 (m, 1H), 7.51 (m, 1H), 7.17 (m, 1H), 7.03 (m, 1H), 6.62 (m, IH), 6.27 (s, 2H), 3.83 (s, 6H); ESI-MS (EI) m/z 41 (M⁺)

### LC-44 (Compound 71)-3-amino-N-(5-methylisoxazol-3-yl)-6-phenyl-4-(trifluoromethyl)thieno[2,3-b]pyridine-2-carboxamide

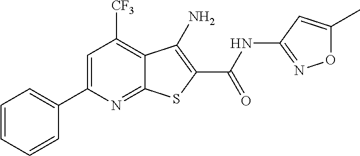 [SMILES: Cc1cc(NC(=O)c2sc3nc(-c4ccccc4)cc(C(F)(F)F)c3c2N)no1]

¹H NMR (CDCl₃, 400 MHz) δ 9.15 (s, 1H), 8.30 (m, 2H), 8.06 (s, 1H), 7.54 (m, 2H), 7.47 (m, 1H), 6.73 (s, 1H), 6.27 (s, 2H), 2.36 (s, 3H); ESI-MS (EI) m/z 418 (M⁺)

### LC-45-ethyl 3-amino-6-(thiophen-2-yl)-4-(trifluoromethyl)thieno[2,3-b]pyridine-2-carboxylate

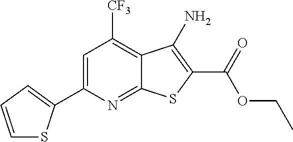 [SMILES: CCOC(=O)c1sc2nc(-c3cccs3)cc(C(F)(F)F)c2c1N]

¹H NMR (CDCl₃, 400 MHz) δ 8.32 (s, 1H), 7.85 (d, 1H), 7.69 (m, 1H), 7.17 (m, 1H), 6.27 (s, 2H), 4.30 (m, 2H), 1.29 (t, 3H); ESI-MS (EI) m/z 372 (M⁺)

All of the methods and apparatuses disclosed and claimed herein can be made and executed without undue experimentation in light of the present disclosure. While the compositions and methods of this invention have been described in terms of preferred embodiments, it will be apparent to those of skill in the art that variations may be applied to the methods and apparatuses and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Alvarez, et al., Science. 327:331, 2010.
Anastassopoulou, et al., Biochemistry. 43:13046, 2004.
Arnold & Sasse, Cancer Res. 21:761, 1961.
Banci, et al., Biochemistry. 47:7423, 2008.
Bertini, et al., Adv Inorg Chem. 45:127, 1998.
Birkaya & Aletta, J Neurobiol. 63:49, 2005.
Boal & Rosenzweig, Chem Rev. 109:4760, 2009.
Brem, S Cancer Control. 6:436, 1999.
Brewer, et al., Clin Cancer Res. 6:1, 2000.
Brewer, J Curr Cancer Drug Targets. 5:195, 2005.
Brewer, J Trace Elements in Exp Med. 16:191, 2003.
Cherny, et al., Neuron. 30:665, 2001.
Cox, et al., Laryngoscope. 111:696, 2001.
Culotta & Gitlin, in Molecular and Metabolic Basis of Inherited Disease, N Y, 2001.
Culotta, et al., Biochim Biophys Acta. 1763:747, 2006.
Daniel, et al., Front Biosci. 9:2652, 2004.
DeNicola, et al., Nature. 475:106-109, 2011.
Finney, et al., Clinical and Experimental Pharmacology and Physiology. 36:88-94, 2009.
Folkman, J Nat Med. 1:27, 1995.
Folkman, N Engl J Med. 285:1182, 1971.
Fotsis, et al., Nature. 368:237, 1994.
Fuchs & de Lustig, Oncology. 46:183, 1989.
Goodman, et al., Endocr Relat Cancer. 11:255, 2004.
Hardie, et al., Nat Rev Mol Cell Biol. 13:251-262, 2012.
Harris, Nutr Rev. 62:60, 2004.
Hitosugi, et al., Med Sci Law. 49:213, 2009.
Hu, J Cell Biochem. 69:326, 1998.
Huang, et al., Nature. 407:390, 2000.
Hung, et al., J Biol Chem. 273:1749, 1998.
Jiang, et al., Hum Mol Genet. 17:3303, 2008.
Khan, et al., Neoplasia. 4(2):164-70, 2002.
Kim, et al., J Nat Chem Biol. 4:176-185, 2008.
Lin, et al., J Biol Chem. 272:9215, 1997.
Linder, in Biochemistry of Copper, New York, 1991.
Lowndes & Harris, J Mammary Gland Biol Neoplasia. 10:299, 2005.
Lutsenko, et al., Arch Biochem Biophys. 476:22, 2008.
Maciag, PNAS USA. 100:6700, 2003.
Mandinov, et al., PNAS USA. 100(11):6700-5, 2003.
Marikovsky, Br J Cancer. 86:779, 2002.
Marikovsky, et al., Int J Cancer. 97:34-41, 2002.
McAuslan & Gole, Trans Ophthalmol Soc UK. 100:354, 1980.
McAuslan & Reilly, W Exp Cell Res. 130:147, 2003.
Menkes, et al., Pediatrics. 29:764, 1962.
Merajver, Clin Cancer Res. 9:1666, 2003.
Merajver, Neoplasia. 4:164, 2002.
Mercer, Am J Clin Nutr. 67:1022S-1028S, 1998.
Mihaylova & Shaw, J Nat Cell Biol. 13:1016-1023, 2011.
Milanino, et al., Agents and Actions. 39:195, 1993.
Muller, et al., Am J Clin Nutr. 67:1082S, 1998.
Muller, et al., Lancet. 347:877, 1996.
Pan, et al., Cancer Res. 62:4854, 2002.
Pena, et al., J Nutr. 129:1251, 1999.
Rae, et al., Science. 284:805-808, 1999.
Raj, et al., Nature. 475:231-234, 2011.
Redman, et al., Neoplasia. 4:164, 2002.
Rivera-Mancia, et al., Chemico-Biological Interactions. 186:184, 2010.
Scheinberg & Sternlieb, Am J Clin Nutr. 63:842S, 1996.
Scott, et al., PLoS One. 7:e29761, 2012.
Sen, et al., Am J Physiol Heart Circ Physiol. 282:H1821, 2002.
Shin, et al., Cancer Informatics. 10:149, 2011.
Sorenson, Am J Clin Nutr. 67:1074S, 1998.
Vinkenborg, et al., Chembiochem. 8:1119, 2007.
Vinkenborg, et al., Nat Methods. 6:737, 2009.
Volker, et al., Atherosclerosis. 130:29, 1997.
Wallace, Nature Reviews Cancer. 12:685-698, 2012.
Walter, et al., Diabetes Care. 14:1050, 1991.
Wernimont, et al., Nat Struct Biol. 7:766, 2000.
Wilson, Brain. 34:295, 1912.
Wood & Thiele, J Biol Chem. 284:404, 2009.
Ziche, et al., J Natl Cancer Inst. 69:475, 1982.

1. A method for treating a patient with cancer comprising administering to the patient a pharmaceutically acceptable composition comprising 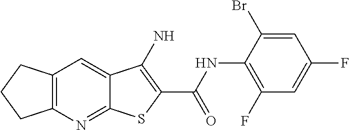 [SMILES: Nc1c(C(=O)Nc2c(F)cc(F)cc2Br)sc2nc3c(cc12)CCC3]
or a pharmaceutically acceptable salt thereof.

2. A method for inhibiting copper trafficking comprising administering to cells of a patient a composition comprising an inhibitor compound having the formula: 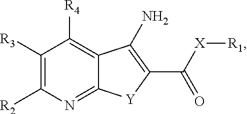
wherein X is NH, O, CH₂, or S,
Y is NH, O or S,
R₁ is hydrogen, substituted or unsubstituted C₁-C₆ alkyl, substituted or unsubstituted, saturated or unsaturated C₃-C₁₅ heterocyclic group, substituted or unsubstituted C₆-C₁₀ aromatic group, or substituted or unsubstituted, saturated or unsaturated C₈-C₁₅ condensed ring group,
R₂ and R₃ are each independently selected from hydrogen, halogen, substituted or unsubstituted, saturated or unsaturated C₃-C₁₅ heterocyclic group, substituted or unsubstituted C₆-C₁₀ aromatic group, or R₂ and R₃ may join together and form substituted or unsubstituted, saturated or unsaturated C₅-C₇ cycloalkyl group, substituted or unsubstituted, saturated or unsaturated C₃-C₁₅ heterocyclic group and
R₄ is hydrogen, halogen, C₁-C₆ alkyl, halogen-substituted C₁-C₆ alkyl, C₁-C₆ alkoxy, halogen-substituted C₁-C₆ alkoxy, cyano, nitro, hydroxyl, substituted amido, or substituted acyl group.

3. The method of claim 2, wherein comprising administering to cells of a patient a composition comprising an inhibitor compound having the formula: 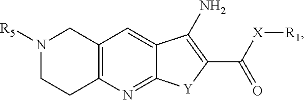
wherein X is NH or O,
Y is O or S,
R₅ is hydrogen, C₁-C₆ alkyl, C₁-C₆ acyl, or —CO₂—C₁-C₆ alkyl, and
R₁ is substituted or unsubstituted, saturated or unsaturated C₃-C₁₅ heterocyclic group, or substituted or unsubstituted C₆-C₁₀ aromatic group.

4. The method of claim 2, wherein comprising administering to cells of a patient a composition comprising an inhibitor compound having the formula: 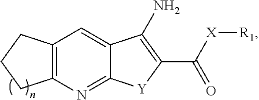
wherein n is 1 or 2,
X is NH or O,
Y is O or S, and
R₁ is substituted or unsubstituted, saturated or unsaturated C₃-C₁₅ heterocyclic group, or substituted or unsubstituted C₆-C₁₀ aromatic group.

5. The method of claim 2, wherein comprising administering to cells of a patient a composition comprising an inhibitor compound having the formula: 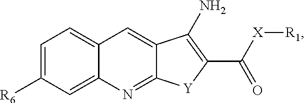
wherein X is NH or O,
Y is O or S,
R₆ is hydrogen, C₁-C₆ alkyl, C₁-C₆ acyl, and C₁-C₆ alkoxy, and
R₁ is substituted or unsubstituted, saturated or unsaturated C₃-C₁₅ heterocyclic group, or substituted or unsubstituted C₆-C₁₀ aromatic group.

6. The method of claim 2, wherein comprising administering to cells of a patient a composition comprising an inhibitor compound having the formula: 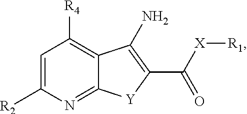
wherein X is NH or O,
Y is O or S,
R₂ is a substituted or unsubstituted, saturated or unsaturated C₃-C₁₅ heterocyclic group, substituted or unsubstituted C₆-C₁₀ aromatic group,
R₁ is C₁-C₆ alkyl, substituted or unsubstituted C₆-C₁₀ aromatic group, and
R₄ is hydrogen, C₁-C₆ alkyl, halogen-substituted C₁-C₆ alkyl, C₁-C₆ alkoxy, halogen-substituted C₁-C₆ alkoxy.

7. The method of claim 2, wherein comprising administering to cells of a patient a composition comprising an inhibitor compound having the formula: 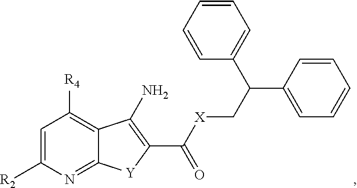
wherein X is NH or O,
Y is O or S,
R₂ is substituted or unsubstituted, saturated or unsaturated C₃-C₁₅ heterocyclic group, or substituted or unsubstituted C₆-C₁₀ aromatic group, and
R₄ is a hydrogen, C₁-C₆ alkyl, halogen-substituted C₁-C₆ alkyl, C₁-C₆ alkoxy or halogen-substituted C₁-C₆ alkoxy.

8. The method of claim 2, wherein comprising administering to cells of a patient a composition comprising an inhibitor compound having the formula: 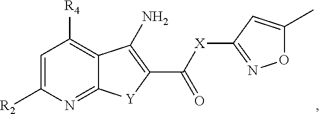
wherein X is NH or O,
Y is O or S,
R₂ is substituted or unsubstituted, saturated or unsaturated C₃-C₁₅ heterocyclic group, or substituted or unsubstituted C₆-C₁₀ aromatic group, and
R₄ is a hydrogen, C₁-C₆ alkyl, halogen-substituted C₁-C₆ alkyl, C₁-C₆ alkoxy or halogen-substituted C₁-C₆ alkoxy.

9.-14. (canceled)

15. The method of claim 2, wherein the cells are cancer cells.

16. The method of claim 15, wherein the cancer cells are tumor cells.

17.-18. (canceled)

19. The method of claim 15, wherein the inhibitor compound is toxic to cancer cells.

20. The method of claim 2, wherein the patient has symptoms of, is at risk for, or has been diagnosed with a copper deficiency disorder or disease.

21. The method of claim 2, wherein the patient has a disease that is Wilson's disease, India childhood cirrhosis, endemic Tyrolean infantile cirrhosis, idiopathic copper toxicosis idiopathic pulmonary fibrosis, liver fibrosis, primary biliary cirrhosis, diabetes mellitus, alzheimer's disease, Huntington's disease, amyotrophic lateral sclerosis, multiple sclerosis, inflammation, or an autoimmune disease.

22. The method of claim 2, wherein administering the composition comprises applying the composition to a wound.

23. The method of claim 2, wherein the inhibitor compound specifically binds to the copper trafficking interface of Atox1 and/or Atox1-like domains that mediate copper trafficking inside mammalian cells.

24. The method of claim 23, wherein binding disrupts simultaneous copper binding.

25. The method of claim 2, wherein the inhibitor compound inhibit cellular copper uptake.

26. The method of claim 2, wherein the composition is administered to a patient intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularly, mucosally, intrapericardially, intraumbilically, intraocularally, orally, topically, locally, by inhalation, by injection, by infusion, by continuous infusion, by localized perfusion bathing target cells directly, via a catheter, or via a lavage.

27. (canceled)

28. The method of claim 2, wherein the composition is a tablet, capsule, liquid, gel, cream, ointment, solution, suspension, emulsion, sustained-release formulation, buccal composition, troche, elixir, syrup, wafer, or combinations thereof.

29. (canceled)

30. A method for treating cancer in a patient comprising administering to the patient an effective amount of a composition comprising an inhibitor compound having the formula: 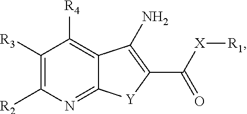
wherein X is NH, O, CH₂, or S,
Y is NH, O or S,
R₁ is hydrogen, substituted or unsubstituted C₁-C₆ alkyl, substituted or unsubstituted, saturated or unsaturated C₃-C₁₅ heterocyclic group, substituted or unsubstituted C₆-C₁₀ aromatic group, substituted or unsubstituted, saturated or unsaturated C₈-C₁₅ condensed ring group,
R₂ and R₃ are each independently selected from hydrogen, halogen, substituted or unsubstituted, saturated or unsaturated C₃-C₁₅ heterocyclic group, substituted or unsubstituted C₆-C₁₀ aromatic group, or R₂ and R₃ may join together and form substituted or unsubstituted, saturated or unsaturated C₅-C₇ cycloalkyl group, substituted or unsubstituted, saturated or unsaturated C₃-C₁₅ heterocyclic group; and
R₄ is hydrogen, halogen, C₁-C₆ alkyl, halogen-substituted C₁-C₆ alkyl, C₁-C₆ alkoxy, halogen-substituted C₁-C₆ alkoxy, cyano, nitro, hydroxyl, substituted amido, or substituted acyl group.

31.-59. (canceled)

60. A pharmaceutical composition for treating and/or preventing cancers, Wilson's disease, India childhood cirrhosis, endemic Tyrolean infantile cirrhosis, idiopathic copper toxicosis idiopathic pulmonary fibrosis, liver fibrosis, primary biliary cirrhosis, diabetes mellitus, alzheimer's disease, huntington's disease, amyotrophic lateral sclerosis, multiple sclerosis, inflammation, and autoimmune diseases comprising a therapeutically effective amount of the compound 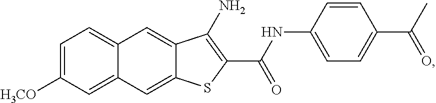 [SMILES: COc1ccc2cc3c(N)c(C(=O)Nc4ccc(C(C)=O)cc4)sc3cc2c1]
or a pharmaceutically acceptable salt thereof.

61.-62. (canceled)
